SEQUENCE LISTING (129 sequences)

SEQ ID NO: 1 - DNA, 189 bp, Homo sapiens
  source 1..189: mol_type = other DNA
cccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctc

SEQ ID NO: 2 - DNA, 43 bp, Homo sapiens
  source 1..43: mol_type = other DNA
ggaaaaacaagggtgggaggacacagcttgtccaaggtcattc

SEQ ID NO: 3 - DNA, 360 bp, Homo sapiens
  source 1..360: mol_type = other DNA
tgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctcct

SEQ ID NO: 4 - DNA, 27 bp, Homo sapiens
  source 1..27: mol_type = other DNA
gaccagcaaactgcctagctgacccca

SEQ ID NO: 5 - DNA, 28 bp, Homo sapiens
  source 1..28: mol_type = other DNA
ggccctgtccccggtgctgaagcgccag

SEQ ID NO: 6 - skipped

SEQ ID NO: 7 - DNA, 13 bp, Mus musculus
  source 1..13: mol_type = other DNA
ctttcaaactcca

SEQ ID NO: 8 - DNA, 74 bp, Mus musculus
  source 1..74: mol_type = other DNA
ttaacccttcccatccgaggagcggctgctgtccgtggtgctgaagcgatagcggcacgggcggctccgtccac

SEQ ID NO: 9 - DNA, 193 bp, Mus musculus
  source 1..193: mol_type = other DNA
ccagccccgcccctctcactgcggagaagccggtcggcccggggccgcgggggaggaggtggagagggtggggccctcctccccagccccccactgccgaggggccggaccgggccaccgcggatataaaagagccggagtcccagagctgccgcagtgctgcctgccccgtcccagccccgcactcccgctc

SEQ ID NO: 10 - DNA, 194 bp, Mus musculus
  source 1..194: mol_type = other DNA
cccagccccgcccctctcactgcggagaagccggtcggcccggggccgcgggggaggaggtggagagggtggggccctcctccccagccccccactgccgaggggccggaccgggccaccgcggatataaaagagccggagtcccagagctgccgcagtgctgcctgccccgtcccagccccgcactcccgctc

SEQ ID NO: 11 - DNA, 194 bp, Macaca mulatta
  source 1..194: mol_type = other DNA
ccctaccccgcccctctcactgcggctgagccggtcagccgggggccgcaggggaggaggcggagagggcggggccctcctccccaccccctcactgacaaggggttggacccggccgcggcggctataaaagggccggcgccctggtgctgccgcagtgcctccagccccgtcccggccccgcgcacctgctc

SEQ ID NO: 12 - DNA, 43 bp, Mus musculus
  source 1..43: mol_type = other DNA
ggaaaaccaaacataggagaacacaatttgtacaaggtcattc

SEQ ID NO: 13 - DNA, 94 bp, Mus musculus
  source 1..94: mol_type = other DNA
tgcatcttgtctcttgcacacaagggaaaaccaaacataggagaacacaatttgtacaaggtcattcagctagcgaagcacagaagctaacccc

SEQ ID NO: 14 - DNA, 43 bp, Macaca mulatta
  source 1..43: mol_type = other DNA
ggaaaaacaagggtgggagaatacagctcgtccaaggtcattc

SEQ ID NO: 15 - DNA, 200 bp, Mus musculus
  source 1..200: mol_type = other DNA
aaatccaagcagtatgggagataaatggggaagccatgtgggcgtaagggggtagaggtctgcatcccagtcccctccccatggcatctgcagtgcctcccagcctttctgacccctgcaaagagcagcatgactggacctttaaattgggaaaatgcttcatcatgttctgctccatcatgaaaaactagagtctcctc

SEQ ID NO: 16 - DNA, 322 bp, Mus musculus
  source 1..322: mol_type = other DNA
tgctgtcaactgcttgtcagacttctcacccccaagaagggcatgtgcattctgcagacaactgaagagactcgaaggaacaagaatctaataacaaaaatccaagcagtatgggagataaatggggaagccatgtgggcgtaagggggtagaggtctgcatcccagtcccctccccatggcatctgcagtgcctcccagcctttctgacccctgcaaagagcagcatgactggacctttaaattgggaaaatgcttcatcatgttctgctccatcatgaaaaactagagtctcctccccctcctccctagtgcactctcct

SEQ ID NO: 17 - DNA, 362 bp, Macaca mulatta
  source 1..362: mol_type = other DNA
tgctgtcagctgcttgtgagccttctcacatccagagaatatatcagcattctgcagaccgaaaagacccagaggaacaaggctccaatggcaaaattccaagtagaatgacaaataaatggggagccatttgagagcaagggagtcctgcccaacaccccctccccatgcctttctcagggacctcagaccagccactcacctccatcctcccagaaccacctgcaaccagcccgttgccccttgcaaactggagcatgactggatctttagatgggggaaaaatgcttcatcatgttctgcttcttcatgcaaaaccagaaactccctccccctcttccctcctcccagcgcactctcct

SEQ ID NO: 18 - skipped

SEQ ID NO: 19 - DNA, 71 bp, Mus musculus
  source 1..71: mol_type = other DNA
gcgctccctttctccgtctgcagtgttctccttctcagggtagctttgcggtcctttcaaactccacgccc

SEQ ID NO: 20 - DNA, 80 bp, Mus musculus
  source 1..80: mol_type = other DNA
atttaacccttcccatccgaggagcggctgctgtccgtggtgctgaagcgatagcggcacgggcggctccgtccactaac

SEQ ID NO: 21 - DNA, 2501 bp, Homo sapiens
  source 1..2501: mol_type = other DNA
cagaatacatcttggagccccctttttaccccaaacccccattcctccttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcaggc

SEQ ID NO: 22 - DNA, 2251 bp, Mus musculus
  source 1..2251: mol_type = other DNA
tgtgctgtcaactgcttgtcagacttctcacccccaagaagggcatgtgcattctgcagacaactgaagagactcgaaggaacaagaatctaataacaaaaatccaagcagtatgggagataaatggggaagccatgtgggcgtaagggggtagaggtctgcatcccagtcccctccccatggcatctgcagtgcctcccagcctttctgacccctgcaaagagcagcatgactggacctttaaattgggaaaatgcttcatcatgttctgctccatcatgaaaaactagagtctcctccccctcctccctagtgcactctcctggcctgcagccaggggctgggaatgagacacaggacaggaaagggatctcttttagggaatctatcagttctcctcctagggatccctccaaaagagaaaaccacagcaaactggggtgcagtgaggcttgaggtaactgcctgggagaagttctgatctgaagaagtctatactggtttccagagcttgtcagtgggcattggagtggggctctctctgctccgggaagaggtttgcagggagaaagaacttcacagagagccaggcactggacaggacatgcaggggtgggtcacttacatacaaccgtaggtcgtttcgagcccgtcatatgactcatccaatcctcccctgtaccgcacagagggactgcttggaaaagctatggaacctccctactccgttaggcatagatttaacccttcccatccgaggagcggctgctgtccgtggtgctgaagcgatagcggcacgggcggctccgtccactaacaccgcttttgaccggaaaaccaaaccaagaacgagccgtataataaagcaagagctccaagtctaagcccctccgccgtccccgccctttcacctgaagcctcagtagggctcatgatggaggtcggtggactttggtactgaaaaaccactccaccacttcctcggagcatgaaaggggatgcttacggcagtactggttcatctattctggaaaaggaatgagatgccaagataaagcagaaaaatcgggcaaggaagggagaaagacaaagttctcaggtgagaggaactggttactattccgactggcaatatgtgggttctcctccccaaaatcagccagacatttcccaagttcgaacctcctaggggcacatgggagcttggagctgcatcttgtctcttgcacacaagggaaaaccaaacataggagaacacaatttgtacaaggtcattcagctagcgaagcacagaagctaaccccaccctgtggcagaacttggcttcggtgttgaggctcttgctgcctactgagggaccccctgttcttcgtaggcagttttcctttccgggcaagaggagactccactttccagtcgtggccactggaatttttagagagcaccacgttcctctcacccagcgctccctttctccgtctgcagtgttctccttctcagggtagctttgcggtcctttcaaactccacgcccaccccaaccccaaccccgaagccagctgtacagttccttaagcccctttgggtggcccagggccgctgtagtatctggggaacactgcaccgccagctagaaggtccccatttatcatcagtagcatccatcatgcaaccccatacagaatcccttcgtgggtgactgcagtctgcactcctcatctcaaggtcctctctaactatcagggaaccaaccctgtgctgcttctcaagtgggggtgtcctctcatagtaatcactgcagtctcccactgcttcaacccgaaggcgccctgacccatcagttctgcaatcctctccctatttccagtgccctctcttattctgagggtcttattctgactaatagggtcttccgacatgcacctggaggtctgcacttgtccgctccggaagtcctttactccttggtctgacctcgggaggctctactgacgatgcgtcgattccccttcactcctgggtcgtcccccccagccccgcccctctcactgcggagaagccggtcggcccggggccgcgggggaggaggtggagagggtggggccctcctccccagccccccactgccgaggggccggaccgggccaccgcggatataaaagagccggagtcccagagctgccgcagtgctgcctgccccgtcccagccccgcactcccgctccgctggcggccgcacctgctccggccatg

SEQ ID NO: 23 - DNA, 2501 bp, Macaca mulatta
  source 1..2501: mol_type = other DNA
cagtccctcttggagccccctttttaccccaaatccctagtcctctttgctgtcagctgcttgtgagccttctcacatccagagaatatatcagcattctgcagaccgaaaagacccagaggaacaaggctccaatggcaaaattccaagtagaatgacaaataaatggggagccatctgagagcaagggagtcctgcccaacaccccctccccatgcctttctcagggacctcagaccagccactcacctccatcctcccagaaccacctgcaaccagcccgttgccccttgcaaactggagcatgactggatctttagatgggggaaaaatgcttcatcatgttctgcttcttcatgcaaaaccagaaactccctccccctcttccctcctcccagcgcactctccttccagtaaaacatggttaaagggacagcgccatcactttcccagctctgagggtctgcttagaaccaggggccttggaaggagacagagggcaaagagaaaggaactggcagaggtctttcctgggggatctgtctgttctgtcctgggaatcctggagcaggaaaactcgggtaaagtgggggtgtagtgggggttgagataaccgcctgggggagattcagagtgcaagtaggagtctacaaactctcaagggggtctcagggctcccggcatccccaggggtcctttcgcaggggtccctatgcaggaggagaacagcccagaaaacagggaactagacccttgacaggaagtccaaggaggggtccctggctcactgtgtgaccctgctggatcactcgcctccgctctcgggtcccctgagcactccgtgcctcccttccctcccctaaagtaaaagcagaagttaatcgctttcccctccccacgcccaacaaagagcaggccctgtccccggtgctgaagcgccagccgcagcgcctcccccactcccaaggcataaaacatgagccaaaaccaataaagaaccaaatgtcacagctgttgcagggccccctaagtcccggggacccctttttctacctgacatcctagtggggtgagggacttttgtacctggaaagcatcccatcacttccctggaagcgagaagggatgccgactcaggcgcctgcttgtctgttatgggggtaggggaccagagaacaagttgaggctgagaagatggggagggggagggagaaaagaggacttcatagtggcgagagaacggcaagatgtgggttccccatccccaattcagccagagacccctcaaagtggaacttcctggggcagtcgggggtcagaagttggagcttgtctctggggcaagacctcttcgttgtacagatggaaaaacaagggtgggagaatacagctcgtccaaggtcattcgactagcaaactgcttagctgaccctagtgtgcagaacctggctcgggtgacacccatcatttccccccaccccacacaggcgccagctctctcaatttcatgctcaagccccgctacggtacccccactgtgggttatctgcccctcaaactcagcccagcttcctcctgcctattcggggaaccctctgcccgcttcgctgagggtccgtcccctttactggggatggcagcagggtctcctgtctcctctctcggggggccactgccgacttttcatagaacgctttgccccctcccaaccccacccatccggggttccctctctccatcctctgcagcgtctcccatacccccattgagggtagtcttggtattctccccaactccaggtcccccttcatctattccagggctggccgcggagtttcctgagcgctctccaagtgggtcctctagatgttaggagaacactgtacttccccccgtcaggggtctcctgtctccgttctatggagcgtccatgctcccattcaggactgtcttgctccctcctctattccggggctggctgcacagtctctgtaccccctatcctgagggcctctcttaactatttggaaagcctcgtgtcctctctcatacggggatcccttcatcctaatgactgcaatcttccattgctccatccctagggcatcctgcccctattcccatcaggtttctccttgtcctctccctgtttcaagtcccctttcttattccgaacacactctcaggctcttccgacgcatacccgggggtcctcactggcccactccgggagtcctctgcccgctaccccgaactcgggggtctcctctgacgcagcgtcgattccccttccctcctcggtcccctaccccgcccctctcactgcggctgagccggtcagccgggggccgcaggggaggaggcggagagggcggggccctcctccccaccccctcactgacaaggggttggacccggccgcggcggctataaaagggccggcgccctggtgctgccgcagtgcctccagccccgtcccggccccgcgcacctgctccggc

SEQ ID NO: 24 - DNA, 1866 bp, Enterobacteria phage lambda
  source 1..1866: mol_type = other DNA
aggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaaccaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctgcttccggtattcgcttaattcagcacaacggaaagagcactggctaaccaggctcgccgactcttcacgattatcgactcaatgctcttacctgttgtgcagatataaaaaatcccgaaaccgttatgcaggctctaactattacctgcgaactgtttcgggattgcattttgcagacctctctgcctgcgatggttggagttccagacgatacgtcgaagtgaccaactaggcggaatcggtagta

SEQ ID NO: 25 - DNA, 20 bp, synthetic construct
  misc_feature 1..20: note = RHO forward primer
  source 1..20: mol_type = other DNA
ctttcctgatctgctgggtg

SEQ ID NO: 26 - DNA, 19 bp, synthetic construct
  misc_feature 1..19: note = RHO reverse primer
  source 1..19: mol_type = other DNA
ggcaaagaacgctgggatg

SEQ ID NO: 27 - DNA, 21 bp, synthetic construct
  misc_feature 1..21: note = EGFP forward primer
  source 1..21: mol_type = other DNA
ttcaagaggacggcaacatcc

SEQ ID NO: 28 - DNA, 22 bp, synthetic construct
  misc_feature 1..22: note = EGFP reverse primer
  source 1..22: mol_type = other DNA
caccttgatgccgttctttcgc

SEQ ID NO: 29 - DNA, 25 bp, synthetic construct
  misc_feature 1..25: note = BETA-actin forward primer
  source 1..25: mol_type = other DNA
tcacccacactgtgcccatctacga

SEQ ID NO: 30 - DNA, 25 bp, synthetic construct
  misc_feature 1..25: note = BETA-actin reverse primer
  source 1..25: mol_type = other DNA
cagcggaaccgctcattgccaatgg

SEQ ID NO: 31 - DNA, 20 bp, synthetic construct
  misc_feature 1..20: note = GAPDH forward primer:
  source 1..20: mol_type = other DNA
cagcctcaagatcatcagca

SEQ ID NO: 32 - DNA, 20 bp, synthetic construct
  misc_feature 1..20: note = GAPDH reverse primer:
  source 1..20: mol_type = other DNA
catgagtccttccacgatac

SEQ ID NO: 33 - DNA, 20 bp, synthetic construct
  misc_feature 1..20: note = Nefh forward primer 1
  source 1..20: mol_type = other DNA
tggccctggacattgagatt

SEQ ID NO: 34 - DNA, 20 bp, synthetic construct
  misc_feature 1..20: note = Nefh reverse primer 1
  source 1..20: mol_type = other DNA
tgcgtggatatggagggaat

SEQ ID NO: 35 - DNA, 20 bp, synthetic construct
  misc_feature 1..20: note = Nefh forward primer 2
  source 1..20: mol_type = other DNA
accgtcatcaggcagacatt

SEQ ID NO: 36 - DNA, 20 bp, synthetic construct
  misc_feature 1..20: note = Nefh reverse primer 2
  source 1..20: mol_type = other DNA
aatgtccagggccatcttga

SEQ ID NO: 37 - DNA, 20 bp, synthetic construct
  misc_feature 1..20: note = Gamma synuclein forward primer
  source 1..20: mol_type = other DNA
tctccattgccaaggaaggt

SEQ ID NO: 38 - DNA, 20 bp, synthetic construct
  misc_feature 1..20: note = Gamma synuclein reverse primer
  source 1..20: mol_type = other DNA
cttgttggccactgtgttga

SEQ ID NO: 39 - DNA, 20 bp, synthetic construct
  misc_feature 1..20: note = Brn3a forward primer
  source 1..20: mol_type = other DNA
cgcagcgtgagaaaatgaac

SEQ ID NO: 40 - DNA, 20 bp, synthetic construct
  misc_feature 1..20: note = Brn3a reverse primer
  source 1..20: mol_type = other DNA
tggcagagaatttcatccgc

SEQ ID NO: 41 - DNA, 20 bp, synthetic construct
  misc_feature 1..20: note = Thy1 forward primer
  source 1..20: mol_type = other DNA
tgaaccaaaaccttcgcctg

SEQ ID NO: 42 - DNA, 20 bp, synthetic construct
  misc_feature 1..20: note = Thy1 reverse primer
  source 1..20: mol_type = other DNA
agctcacaaaagtagtcgcc

SEQ ID NO: 43 - DNA, 20 bp, synthetic construct
  misc_feature 1..20: note = Melanopsin forward primer
  source 1..20: mol_type = other DNA
gggttctgagagtgaagtgg

SEQ ID NO: 44 - DNA, 19 bp, synthetic construct
  misc_feature 1..19: note = Melanopsin reverse primer
  source 1..19: mol_type = other DNA
aagaggccttgagttctcc

SEQ ID NO: 45 - DNA, 38 bp, synthetic construct
  misc_feature 1..38: note = pAAV.NEFH-EGFP forward primer
  source 1..38: mol_type = other DNA
agatcatcttaagacgcgttgctgtcagctgcttgtga

SEQ ID NO: 46 - DNA, 21 bp, synthetic construct
  misc_feature 1..21: note = pAAV.NEFH-EGFP reverse primer
  source 1..21: mol_type = other DNA
gaggtacagtgttctcctaac

SEQ ID NO: 47 - DNA, 695 bp, synthetic construct
  misc_feature 1..695: note = NEFH
  source 1..695: mol_type = other DNA
agtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcagcgatatcctaggaattcagcttctaga

SEQ ID NO: 48 - DNA, 32 bp, synthetic construct
  misc_feature 1..32: note = pAAV.A-EGFP forward primer
  source 1..32: mol_type = other DNA
atcgatgacgcgtctctgacgcagcgtcgatt

SEQ ID NO: 49 - DNA, 32 bp, synthetic construct
  misc_feature 1..32: note = pAAV.A-EGFP reverse primer
  source 1..32: mol_type = other DNA
agatcatgatatcggcctgagcaggtgcgcga

SEQ ID NO: 50 - DNA, 640 bp, synthetic construct
  misc_feature 1..640: note = Conserved Region A+F
  source 1..640: mol_type = other DNA
agagatcatacgcgtctagtcatctcagttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaagtttaaacttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctctcacgtgatcagagatatctcagaca

SEQ ID NO: 51 - DNA, 34 bp, synthetic construct
  misc_feature 1..34: note = pAAV.A-spacer-F-EGFP forward primer
  source 1..34: mol_type = other DNA
atcgatgtttaaactactaccgattccgcctagt

SEQ ID NO: 52 - DNA, 34 bp, synthetic construct
  misc_feature 1..34: note = pAAV.A-spacer-F-EGFP reverse primer
  source 1..34: mol_type = other DNA
atgcatgtttaaacaggcatttatactccgctgg

SEQ ID NO: 53 - DNA, 133 bp, synthetic construct
  misc_feature 1..133: note = AAV.A+F-EGFP L-ITR
  source 1..133: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 54 - DNA, 14 bp, synthetic construct
  misc_feature 1..14: note = AAV.A+F-EGFP plasmid
  source 1..14: mol_type = other DNA
gcggccgcacgcgt

SEQ ID NO: 55 - DNA, 14 bp, synthetic construct
  misc_feature 1..14: note = AAV.A+F-EGFP spacer
  source 1..14: mol_type = other DNA
ctagtcatctcagt

SEQ ID NO: 56 - DNA, 360 bp, synthetic construct; identical to SEQ ID NO: 3
  misc_feature 1..360: note = AAV.A+F-EGFP Conserved region F
  source 1..360: mol_type = other DNA

SEQ ID NO: 57 - DNA, 36 bp, synthetic construct
  misc_feature 1..36: note = AAV.A+F-EGFP spacer
  source 1..36: mol_type = other DNA
tccagtaagtttaaacttccctcctcggtcccctgc

SEQ ID NO: 58 - DNA, 195 bp, synthetic construct
  misc_feature 1..195: note = AAV.A+F-EGFP Conserved region A
  source 1..195: mol_type = other DNA
ccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctc

SEQ ID NO: 59 - DNA, 73 bp, synthetic construct
  misc_feature 1..73: note = AAV.A+F-EGFP spacer plasmid
  source 1..73: mol_type = other DNA
tcacgtgatcagagataattccccggggatcctctagagtcgacctgcagaagctgatccaccggtcgccacc

SEQ ID NO: 60 - DNA, 718 bp, synthetic construct
  misc_feature 1..718: note = AAV.A+F-EGFP EGFP
  source 1..718: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagt

SEQ ID NO: 61 - DNA, 42 bp, synthetic construct
  misc_feature 1..42: note = AAV.A+F-EGFP plasmid
  source 1..42: mol_type = other DNA
aaagcggccagcttgcctcgagcagcgctgctcgagagatct

SEQ ID NO: 62 - DNA, 479 bp, synthetic construct
  misc_feature 1..479: note = AAV.A+F-EGFP hGH_PA_terminator
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 63 - DNA, 39 bp, synthetic construct
  misc_feature 1..39: note = AAV.A+F-EGFP plasmid
  source 1..39: mol_type = other DNA
ctgattttgtaggtaaccacgtgcggaccgagcggccgc

SEQ ID NO: 64 - DNA, 140 bp, synthetic construct
  misc_feature 1..140: note = AAV.A+F-EGFP R-ITR
  source 1..140: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgagg

SEQ ID NO: 65 - DNA, 2605 bp, synthetic construct
  misc_feature 1..2605: note = AAV.A+F-EGFP plasmid backbone
  source 1..2605: mol_type = other DNA
ggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 66 - DNA, 133 bp, synthetic construct; identical to SEQ ID NO: 53
  misc_feature 1..133: note = AAV.CMV-EGFP L-ITR
  source 1..133: mol_type = other DNA

SEQ ID NO: 67 - DNA, 14 bp, synthetic construct
  misc_feature 1..14: note = AAV.CMV-EGFP plasmid
  source 1..14: mol_type = other DNA
gcggccgcacgcgt

SEQ ID NO: 68 - DNA, 664 bp, synthetic construct
  misc_feature 1..664: note = AAV.CMV-EGFP CMV promoter
  source 1..664: mol_type = other DNA
ggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggatt

SEQ ID NO: 69 - DNA, 499 bp, synthetic construct
  misc_feature 1..499: note = AAV.CMV-EGFP beta-globin intron
  source 1..499: mol_type = other DNA
cgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaac

SEQ ID NO: 70 - DNA, 63 bp, synthetic construct
  misc_feature 1..63: note = AAV.CMV-EGFP MCS
  source 1..63: mol_type = other DNA
atcgattgaattccccggggatcctctagagtcgacctgcaagctgatccaccggtcgccacc

SEQ ID NO: 71 - DNA, 718 bp, synthetic construct; identical to SEQ ID NO: 60
  misc_feature 1..718: note = AAV.CMV-EGFP EGFP
  source 1..718: mol_type = other DNA

SEQ ID NO: 72 - DNA, 42 bp, synthetic construct; identical to SEQ ID NO: 61
  misc_feature 1..42: note = AAV.CMV-EGFP plasmid
  source 1..42: mol_type = other DNA

SEQ ID NO: 73 - DNA, 479 bp, synthetic construct; identical to SEQ ID NO: 62
  misc_feature 1..479: note = AAV.CMV-EGFP hGH_PA_terminator
  source 1..479: mol_type = other DNA

SEQ ID NO: 74 - DNA, 39 bp, synthetic construct; identical to SEQ ID NO: 63
  misc_feature 1..39: note = AAV.CMV-EGFP plasmid
  source 1..39: mol_type = other DNA

SEQ ID NO: 75 - DNA, 140 bp, synthetic construct; identical to SEQ ID NO: 64
  misc_feature 1..140: note = AAV.CMV-EGFP R-ITR
  source 1..140: mol_type = other DNA

SEQ ID NO: 76 - DNA, 2605 bp, synthetic construct; identical to SEQ ID NO: 65
  misc_feature 1..2605: note = AAV.CMV-EGFP Plasmid backbone
  source 1..2605: mol_type = other DNA

SEQ ID NO: 77 - DNA, 133 bp, synthetic construct; identical to SEQ ID NO: 53
  misc_feature 1..133: note = AAV.A-EGFP L-ITR
  source 1..133: mol_type = other DNA

SEQ ID NO: 78 - DNA, 14 bp, synthetic construct
  misc_feature 1..14: note = AAV.A-EGFP plasmid
  source 1..14: mol_type = other DNA
gcggccgcacgcgt

SEQ ID NO: 79 - DNA, 37 bp, synthetic construct
  misc_feature 1..37: note = AAV.A-EGFP spacer
  source 1..37: mol_type = other DNA
ctctgacgcagcgtcgattccccttccctcctcggtc

SEQ ID NO: 80 - DNA, 195 bp, synthetic construct; identical to SEQ ID NO: 58
  misc_feature 1..195: note = AAV.A-EGFP Conserved region A
  source 1..195: mol_type = other DNA

SEQ ID NO: 81 - DNA, 65 bp, synthetic construct
  misc_feature 1..65: note = AAV.A-EGFP plasmid spacer
  source 1..65: mol_type = other DNA
aggccgataattccccggggatcctctagagtcgacctgcagaagctgatccaccggtcgccacc

SEQ ID NO: 82 - DNA, 718 bp, synthetic construct; identical to SEQ ID NO: 60
  misc_feature 1..718: note = AAV.A-EGFP EGFP
  source 1..718: mol_type = other DNA

SEQ ID NO: 83 - DNA, 42 bp, synthetic construct; identical to SEQ ID NO: 61
  misc_feature 1..42: note = AAV.A-EGFP plasmid
  source 1..42: mol_type = other DNA

SEQ ID NO: 84 - DNA, 479 bp, synthetic construct; identical to SEQ ID NO: 62
  misc_feature 1..479: note = AAV.A-EGFP hGH_PA_terminator
  source 1..479: mol_type = other DNA

SEQ ID NO: 85 - DNA, 39 bp, synthetic construct; identical to SEQ ID NO: 63
  misc_feature 1..39: note = AAV.A-EGFP plasmid
  source 1..39: mol_type = other DNA

SEQ ID NO: 86 - DNA, 140 bp, synthetic construct; identical to SEQ ID NO: 64
  misc_feature 1..140: note = AAV.A-EGFP R-ITR
  source 1..140: mol_type = other DNA

SEQ ID NO: 87 - DNA, 2605 bp, synthetic construct; identical to SEQ ID NO: 65
  misc_feature 1..2605: note = AAV.A-EGFP Plasmid backbone
  source 1..2605: mol_type = other DNA

SEQ ID NO: 88 - DNA, 133 bp, synthetic construct; identical to SEQ ID NO: 53
  misc_feature 1..133: note = AAV.A-spacer-F-EGFP L-ITR
  source 1..133: mol_type = other DNA

SEQ ID NO: 89 - DNA, 28 bp, synthetic construct
  misc_feature 1..28: note = AAV.A-spacer-F-EGFP plasmid
  source 1..28: mol_type = other DNA
gcggccgcacgcgtctagtcatctcagt

SEQ ID NO: 90 - DNA, 360 bp, synthetic construct; identical to SEQ ID NO: 3
  misc_feature 1..360: note = AAV.A-spacer-F-EGFP Conserved region F
  source 1..360: mol_type = other DNA

SEQ ID NO: 91 - DNA, 16 bp, synthetic construct
  misc_feature 1..16: note = AAV.A-spacer-F-EGFP plasmid
  source 1..16: mol_type = other DNA
tccagtaagtttaaac

SEQ ID NO: 92 - DNA, 1866 bp, synthetic construct; identical to SEQ ID NO: 24
  misc_feature 1..1866: note = AAV.A-spacer-F-EGFP spacer
  source 1..1866: mol_type = other DNA

SEQ ID NO: 93 - DNA, 22 bp, synthetic construct
  misc_feature 1..22: note = AAV.A-spacer-F-EGFP plasmid
  source 1..22: mol_type = other DNA
gtttaaacttccctcctcggtc

SEQ ID NO: 94 - DNA, 195 bp, synthetic construct; identical to SEQ ID NO: 58
  misc_feature 1..195: note = AAV.A-spacer-F-EGFP Conserved region A
  source 1..195: mol_type = other DNA

SEQ ID NO: 95 - DNA, 72 bp, synthetic construct
  misc_feature 1..72: note = AAV.A-spacer-F-EGFP plasmid
  source 1..72: mol_type = other DNA
tcacgtgatcagagataattccccggggatcctctagagcgacgtgcagaagctgatccaccggtcgccacc

SEQ ID NO: 96 - DNA, 718 bp, synthetic construct; identical to SEQ ID NO: 60
  misc_feature 1..718: note = AAV.A-spacer-F-EGFP EGFP
  source 1..718: mol_type = other DNA

SEQ ID NO: 97 - DNA, 42 bp, synthetic construct; identical to SEQ ID NO: 61
  misc_feature 1..42: note = AAV.A-spacer-F-EGFP plasmid
  source 1..42: mol_type = other DNA

SEQ ID NO: 98 - DNA, 479 bp, synthetic construct; identical to SEQ ID NO: 62
  misc_feature 1..479: note = AAV.A-spacer-F-EGFP hGH_PA_terminator
  source 1..479: mol_type = other DNA

SEQ ID NO: 99 - DNA, 39 bp, synthetic construct; identical to SEQ ID NO: 63
  misc_feature 1..39: note = AAV.A-spacer-F-EGFP Plasmid
  source 1..39: mol_type = other DNA

SEQ ID NO: 100 - DNA, 140 bp, synthetic construct; identical to SEQ ID NO: 64
  misc_feature 1..140: note = AAV.A-spacer-F-EGFP R-ITR
  source 1..140: mol_type = other DNA

SEQ ID NO: 101 - DNA, 2605 bp, synthetic construct; identical to SEQ ID NO: 65
  misc_feature 1..2605: note = AAV.A-spacer-F-EGFP Plasmid backbone
  source 1..2605: mol_type = other DNA

SEQ ID NO: 102 - DNA, 133 bp, synthetic construct; identical to SEQ ID NO: 53
  misc_feature 1..133: note = AAV.huNEFH-EGFP L-ITR
  source 1..133: mol_type = other DNA

SEQ ID NO: 103 - DNA, 24 bp, synthetic construct
  misc_feature 1..24: note = AAV.huNEFH-EGFP Plasmid/spacer
  source 1..24: mol_type = other DNA
gcggccgcacgcgttaagacgcgt

SEQ ID NO: 104 - DNA, 2448 bp, synthetic construct
  misc_feature 1..2448: note = AAV.huNEFH-EGFP huNEFH with conserved regions F, D and A highlighted
  source 1..2448: mol_type = other DNA
tgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctc

SEQ ID NO: 105 - DNA, 66 bp, synthetic construct
  misc_feature 1..66: note = AAV.huNEFH-EGFP Spacer/plasmid
  source 1..66: mol_type = other DNA
agcgatatcctaggaattccccatcctctagagtcgacctgcagaagctatccaccggtcgccacc

SEQ ID NO: 106 - DNA, 718 bp, synthetic construct; identical to SEQ ID NO: 60
  misc_feature 1..718: note = AAV.huNEFH-EGFP EGFP
  source 1..718: mol_type = other DNA

SEQ ID NO: 107 - DNA, 42 bp, synthetic construct; identical to SEQ ID NO: 61
  misc_feature 1..42: note = AAV.huNEFH-EGFP plasmid
  source 1..42: mol_type = other DNA

SEQ ID NO: 108 - DNA, 479 bp, synthetic construct; identical to SEQ ID NO: 62
  misc_feature 1..479: note = AAV.huNEFH-EGFP hGH_PA_terminator
  source 1..479: mol_type = other DNA

SEQ ID NO: 109 - DNA, 39 bp, synthetic construct; identical to SEQ ID NO: 63
  misc_feature 1..39: note = AAV.huNEFH-EGFP Plasmid
  source 1..39: mol_type = other DNA

SEQ ID NO: 110 - DNA, 140 bp, synthetic construct; identical to SEQ ID NO: 64
  misc_feature 1..140: note = AAV.huNEFH-EGFP R-ITR
  source 1..140: mol_type = other DNA

SEQ ID NO: 111 - DNA, 2605 bp, synthetic construct; identical to SEQ ID NO: 65
  misc_feature 1..2605: note = AAV.huNEFH-EGFP Plasmid backbone
  source 1..2605: mol_type = other DNA

SEQ ID NO: 112 - DNA, 133 bp, synthetic construct; identical to SEQ ID NO: 53
  misc_feature 1..133: note = AAV.muNefh-EGFP L-ITR
  source 1..133: mol_type = other DNA

SEQ ID NO: 113 - DNA, 69 bp, synthetic construct
  misc_feature 1..69: note = AAV.muNefh-EGFP plasmid
  source 1..69: mol_type = other DNA
gcggccgcacgcgttaagacgcgtgccacaccttttaccttcacacacaccccactcctatcattcatg

SEQ ID NO: 114 - DNA, 2251 bp, synthetic construct; identical to SEQ ID NO: 22
  misc_feature 1..2251: note = AAV.muNefh-EGFP muNefh
  source 1..2251: mol_type = other DNA

SEQ ID NO: 115 - DNA, 94 bp, synthetic construct
  misc_feature 1..94: note = AAV.muNefh-EGFP muNefh plasmid
  source 1..94: mol_type = other DNA
cccgtcccagccccgcactcccgctccgctggcggccgcacctgctccggccattctagagtcgacctgcagaagctatccaccggtcgccacc

SEQ ID NO: 116 - DNA, 718 bp, synthetic construct; identical to SEQ ID NO: 60
  misc_feature 1..718: note = AAV.muNefh-EGFP EGFP
  source 1..718: mol_type = other DNA

SEQ ID NO: 117 - DNA, 42 bp, synthetic construct; identical to SEQ ID NO: 61
  misc_feature 1..42: note = AAV.muNefh-EGFP EGFP plasmid
  source 1..42: mol_type = other DNA

SEQ ID NO: 118 - DNA, 479 bp, synthetic construct; identical to SEQ ID NO: 62
  misc_feature 1..479: note = AAV.muNefh-EGFP hGH_PA_terminator
  source 1..479: mol_type = other DNA

SEQ ID NO: 119 - DNA, 39 bp, synthetic construct; identical to SEQ ID NO: 63
  misc_feature 1..39: note = AAV.muNefh-EGFP hGH_PA_terminator plasmid
  source 1..39: mol_type = other DNA

SEQ ID NO: 120 - DNA, 140 bp, synthetic construct; identical to SEQ ID NO: 64
  misc_feature 1..140: note = AAV.muNefh-EGFP R-ITR
  source 1..140: mol_type = other DNA

SEQ ID NO: 121 - DNA, 2605 bp, synthetic construct; identical to SEQ ID NO: 65
  misc_feature 1..2605: note = AAV.muNefh-EGFP Plasmid backbone
  source 1..2605: mol_type = other DNA

SEQ ID NO: 122 - DNA, 20 bp, synthetic construct
  misc_feature 1..20: note = Thy1 Forward primer
  source 1..20: mol_type = other DNA
tgaaccaaaaccttcgcctg

SEQ ID NO: 123 - DNA, 20 bp, synthetic construct
  misc_feature 1..20: note = Thy1 reverse primer
  source 1..20: mol_type = other DNA
agctcacaaaagtagtcgcc

SEQ ID NO: 124 - DNA, 4467 bp, synthetic construct
  misc_feature 1..4467: note = AAV.A-EGFP
  source 1..4467: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcaggccgataattccccggggatcctctagagtcgacctgcagaagctgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 125 - DNA, 6715 bp, synthetic construct
  misc_feature 1..6715: note = AAV.A-spacer-F-EGFP
  source 1..6715: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctagtcatctcagttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaagtttaaacaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaaccaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctgcttccggtattcgcttaattcagcacaacggaaagagcactggctaaccaggctcgccgactcttcacgattatcgactcaatgctcttacctgttgtgcagatataaaaaatcccgaaaccgttatgcaggctctaactattacctgcgaactgtttcgggattgcattttgcagacctctctgcctgcgatggttggagttccagacgatacgtcgaagtgaccaactaggcggaatcggtagtagtttaaacttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctctcacgtgatcagagataattccccggggatcctctagagcgacgtgcagaagctgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 126 - DNA, 4848 bp, synthetic construct
  misc_feature 1..4848: note = AAV.A+F-EGFP
  source 1..4848: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctagtcatctcagttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaagtttaaacttccctcctcggtcccctgcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctctcacgtgatcagagataattccccggggatcctctagagtcgacctgcagaagctgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 127 - DNA, 5396 bp, synthetic construct
  misc_feature 1..5396: note = AAV.CMV-EGFP
  source 1..5396: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatcctctagagtcgacctgcaagctgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 128 - DNA, 6694 bp, synthetic construct
  misc_feature 1..6694: note = AAV.huNEFH-EGFP
  source 1..6694: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaagacgcgttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcagcgatatcctaggaattccccatcctctagagtcgacctgcagaagctatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

SEQ ID NO: 129 - DNA, 6570 bp, synthetic construct
  misc_feature 1..6570: note = AAV.muNefh-EGFP
  source 1..6570: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaagacgcgtgccacaccttttaccttcacacacaccccactcctatcattcatgtgtgctgtcaactgcttgtcagacttctcacccccaagaagggcatgtgcattctgcagacaactgaagagactcgaaggaacaagaatctaataacaaaaatccaagcagtatgggagataaatggggaagccatgtgggcgtaagggggtagaggtctgcatcccagtcccctccccatggcatctgcagtgcctcccagcctttctgacccctgcaaagagcagcatgactggacctttaaattgggaaaatgcttcatcatgttctgctccatcatgaaaaactagagtctcctccccctcctccctagtgcactctcctggcctgcagccaggggctgggaatgagacacaggacaggaaagggatctcttttagggaatctatcagttctcctcctagggatccctccaaaagagaaaaccacagcaaactggggtgcagtgaggcttgaggtaactgcctgggagaagttctgatctgaagaagtctatactggtttccagagcttgtcagtgggcattggagtggggctctctctgctccgggaagaggtttgcagggagaaagaacttcacagagagccaggcactggacaggacatgcaggggtgggtcacttacatacaaccgtaggtcgtttcgagcccgtcatatgactcatccaatcctcccctgtaccgcacagagggactgcttggaaaagctatggaacctccctactccgttaggcatagatttaacccttcccatccgaggagcggctgctgtccgtggtgctgaagcgatagcggcacgggcggctccgtccactaacaccgcttttgaccggaaaaccaaaccaagaacgagccgtataataaagcaagagctccaagtctaagcccctccgccgtccccgccctttcacctgaagcctcagtagggctcatgatggaggtcggtggactttggtactgaaaaaccactccaccacttcctcggagcatgaaaggggatgcttacggcagtactggttcatctattctggaaaaggaatgagatgccaagataaagcagaaaaatcgggcaaggaagggagaaagacaaagttctcaggtgagaggaactggttactattccgactggcaatatgtgggttctcctccccaaaatcagccagacatttcccaagttcgaacctcctaggggcacatgggagcttggagctgcatcttgtctcttgcacacaagggaaaaccaaacataggagaacacaatttgtacaaggtcattcagctagcgaagcacagaagctaaccccaccctgtggcagaacttggcttcggtgttgaggctcttgctgcctactgagggaccccctgttcttcgtaggcagttttcctttccgggcaagaggagactccactttccagtcgtggccactggaatttttagagagcaccacgttcctctcacccagcgctccctttctccgtctgcagtgttctccttctcagggtagctttgcggtcctttcaaactccacgcccaccccaaccccaaccccgaagccagctgtacagttccttaagcccctttgggtggcccagggccgctgtagtatctggggaacactgcaccgccagctagaaggtccccatttatcatcagtagcatccatcatgcaaccccatacagaatcccttcgtgggtgactgcagtctgcactcctcatctcaaggtcctctctaactatcagggaaccaaccctgtgctgcttctcaagtgggggtgtcctctcatagtaatcactgcagtctcccactgcttcaacccgaaggcgccctgacccatcagttctgcaatcctctccctatttccagtgccctctcttattctgagggtcttattctgactaatagggtcttccgacatgcacctggaggtctgcacttgtccgctccggaagtcctttactccttggtctgacctcgggaggctctactgacgatgcgtcgattccccttcactcctgggtcgtcccccccagccccgcccctctcactgcggagaagccggtcggcccggggccgcgggggaggaggtggagagggtggggccctcctccccagccccccactgccgaggggccggaccgggccaccgcggatataaaagagccggagtcccagagctgccgcagtgctgcctgccccgtcccagccccgcactcccgctccgctggcggccgcacctgctccggccatgcccgtcccagccccgcactcccgctccgctggcggccgcacctgctccggccattctagagtcgacctgcagaagctatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcagg

(19) United States
(12) Patent Application Publication
(10) Pub. No.: US 2026/0345828 A1
(43) Pub. Date: Nov. 5, 2026

(54) Retinal Promoter and Uses Thereof

(71) Applicant: The Provost, Fellows and Scholars of the College of the Holy and Undivided Trinity of Queen Elizabet, Dublin (IE)

(72) Inventors: Naomi Chadderton, Dublin (IE); Gwenyth Jane Farrar, Dublin (IE); Killian Hanlon, Dublin (IE); Paul F. Kenna, Dublin (IE); Arpad Palfi, Dublin (IE); Sophia Millington Ward, Dublin (IE)

(21) Appl. No.: 19/406,692

(22) Filed: Dec. 2, 2025

Related U.S. Application Data
(62) Division of application No. 16/643,534, filed on Sep. 3, 2018, now Pat. No. 12,527,883.

(30) Foreign Application Priority Data
Sep. 1, 2017 (GB) 1714066.6

(51) Int. Cl. A61K 48/00; A61P 27/02; C12N 15/86

(52) U.S. Cl. CPC A61K 48/0058; A61K 48/0075; A61P 27/02; C12N 15/86; C12N 2750/14143; C12N 2830/008

(57) ABSTRACT

Disclosed is a promoter for driving expression in the retina. The promoter sequence comprises at least NEFH promoter conserved region A and optionally one or more of NEFH conserved regions D, F, D1, K, B, C and E. Also disclosed are uses of the promoter for directing expression to retinal ganglion cells and uses for the treatment of ocular diseases.

### CROSS REFERENCE TO RELATED APPLICATIONS

This application is a divisional application of U.S. patent application Ser. No. 16/643,534, filed on Feb. 29, 2020, which is a US national phase of International Application No. PCT/FR2017/052476, filed on Sep. 15, 2017, which claims benefit from FR Patent Application No. 1658666, filed on Sep. 15, 2016, the entire contents of each of which are incorporated herein by reference.

### REFERENCE TO SEQUENCE LISTING SUBMITTED ELECTRONICALLY

This application contains a sequence listing which is submitted electronically and is hereby incorporated by reference in its entirety. The sequence listing submitted herewith is contained in the XML filed created Dec. 2, 2025, entitled “20-384-WO-US-DIV_Sequence-Listing.xml” and is 234,718 bytes in size.

### FIELD OF THE INVENTION

The invention relates to promoters, in particular promoters for driving expression of genes in the retina. The invention further relates to vectors comprising such promoters and the use of said promoters and vectors in medical treatments, in particular in the treatment of conditions and diseases of the eye. In addition the invention relates to the use of said promoters and vectors in methods such as to drive the expression of marker genes, e.g., EGFP in retina.

### BACKGROUND OF THE INVENTION

In recent years significant progress has been made in gene therapy with the market authorisation of therapies such as Glybera®, T-VEC (Imlygic™) and Strimvelis™. Many more potential gene therapies are currently in later phase clinical trials (clinicaltrials.gov) and it is likely that the rate of clinical development will continue to increase. Advances in our understanding of viral vectors has allowed for the generation of a range of delivery vehicles that can collectively target a wide array of cell types.

The retina in particular has been the focus of many gene therapy studies. The retina is a confined but readily accessible target, and retinal neurons are non-dividing in mammals—thus a gene therapy can in theory provide long-term benefit. Furthermore the retina is immune privileged and therefore, in principle, may be more tolerant of treatments. Indeed, many gene therapy clinical trials have been completed or are ongoing in the retina (clinicaltrials.gov). Adeno-associated virus (AAV) has been the vehicle of choice for the majority of retinal gene therapy studies as it achieves efficient neuronal transduction, provides long-term expression in terminally differentiated cells and has demonstrated a good safety profile in humans (Bainbridge et al., 2015; Bennett et al., 2016; Feuer et al., 2015; Ghazi et al., 2016; Hauswirth et al., 2008; MacLaren et al., 2014; Russell et al., 2017; clinicaltrials.gov). The successful completion of a Phase III trial to treat RPE65-linked inherited retinal degenerations, such as Leber congenital amaurosis (LCA; sparktx.com; Russell et al., 2017) represents a significant milestone in the field.

Many therapeutic studies to date targeting the retina have been directed towards photoreceptor cells and the retinal pigment epithelium (RPE). Approximately 1/3000 people worldwide suffer from an inherited retinal degeneration (IRD); many of these are caused by mutations directly or indirectly affecting photoreceptors (Bessant et al., 2001). However, retinal disorders involving the ganglion cell layer (GCL) should in theory also be readily amenable to gene therapy, given efficient cell targeting. Intravitreal injection of AAV for delivery to GCL typically involves less surgical trauma than subretinal injection. Notably, anti-VEGF treatments, such as Lucentis, are routinely administered intravitreally to age related macular degeneration (AMD) patients.

Limiting expression of a gene therapy to a target cell type is often preferable, and in principle represents a valuable safety feature. Although AAV-mediated ocular gene therapy has been shown to be well-tolerated (Bainbridge et al., 2015; Bennett et al., 2016; Feuer et al., 2015; Ghazi et al., 2016; Hauswirth et al., 2008; MacLaren et al., 2014; Russell et al., 2017) directing transgene expression to the target cells of interest may reduce the chance of immune response(s) or other unwanted off-target effects, thus providing a more efficacious therapy. There are approximately 1.5 million retinal ganglion cells (RGCs) in the human retina, comprising approximately 1% of retinal neurons, (Callaway, 2005) and composed of over thirty different classes of cells (Baden et al., 2016; Masland, 2012). However, knowledge regarding the different types of RGCs populating the GCL is still emerging. A number of gene therapies have been directed towards RGCs, with several ongoing or completed clinical trials (Feuer et al., 2016; Yang et al., 2016; clinicaltrials.gov).

Such therapies have typically utilised ubiquitous promoters such as cytomegalovirus (CMV) or chicken-β-actin (CBA; Bennett et al., 2016; Boye et al., 2010; Feuer et al., 2016; Koilkonda et al., 2010). These promoters typically offer high levels of expression, and tend to be small in size, which is valuable as the packaging capacity of AAV is limited to approximately 2-5 kb, with an optimum at 4.7 kb (Dong et al., 1996; Grieger and Samulski, 2005). However, a significant disadvantage of generic promoters is that, they may drive gene expression in cell types other than the target cells.

Cell-type specific promoters such as rhodopsin (Bennett et al., 1998; Flannery et al., 1997; O'Reilly et al., 2007; Palfi et al., 2010; Wert et al., 2013), rhodopsin kinase (Boye et al., 2010; Kay et al., 2013; Khani et al., 2007; Molday et al., 2013; Sun et al., 2010), RPE65 (Bainbridge et al., 2008, 2015) and retinaldehyde binding protein 1 (RLBP1; Choi et al., 2015), among others, have successfully been used in retinal gene therapy approaches. Preferential RGC expression in transgenic animal models has been achieved using the Thy1 promoter, which confers expression that is thought to be limited to RGCs. It has been shown that an enhancer element contained in the first intron of Thy1 is necessary for both high level and specific gene expression (Aliç et al., 2016; Spanopoulou et al., 1991). However, while the core promoter and enhancer element are both small (~100-200 bp each), approximately 6 kb of spacing between the two elements is believed to be necessary for specific promoter function, making the Thy1 promoter unsuitable for use in AAV vectors. A 0.48 kb promoter derived from the human synapsin-1 gene (hSYN) can provide pan-neuronal expression in rodent and primate brains when utilised in adenoviral or AAV vectors (Diester et al., 2011; Kügler et al., 2003a, 2003b; Lopez et al., 2016). In the rodent retina, intravitreal injection of an AAV gene construct driven by hSYN resulted in expression in the GCL (Gaub et al., 2014). However, in the context of the primate retina, hSYN promoter-mediated expression only appears to occur in damaged retinas or vitreolysed eyes (Yin et al., 2011). The therapeutic relevance of the hSYN promoter therefore remains to be fully established. Hence, the characterisation of a promoter that exhibits preferential RGC expression and may be used reliably in gene therapy of the eye would represent a significant refinement for RGC gene therapies. Such a promoter would be of particular value if it were sufficiently small for use in AAV vectors.

### SUMMARY OF THE INVENTION

The present invention addresses some of the problems of the prior art. The inventors have performed extensive studies to identify suitable promoters for use in gene therapy of the eye. They have developed and refined a number of criteria to identify suitable candidate promoters to drive preferential gene expression in RGCs for use in gene therapy of the eye or for use to drive marker gene expression in RGCs. Having identified a candidate promoter sequence the inventors established its suitability experimentally in vivo. Initially, GCL-specific microarray expression data from post-mortem human retinas was used (Kim et al., 2006). In this paper, Kim et al. describe the isolation of GCL populations consisting of 1,000 RGCs using laser-capture microdissection (LCM) and cell populations consisting of 1,000 cells from the remainder of the retina (termed outer retina, OR) and the comparison of gene expression between the two populations. Using these data, the present inventors have assessed promoter conservation between mammalian species for genes that were highly expressed and enriched in RGCs, using data drawn from the UCSC database (mm10; Kent et al., 2002). Conservation of non-coding DNA sequence across species was used as an indicator of potential function, and a number of highly conserved promoter upstream sequences were identified from genes shown to be both highly expressed and enriched in RGCs (Choudhury et al., 2016; Kim et al., 2006; Struebing et al., 2016). Candidate promoters were evaluated and compared to CMV-driven gene expression in RGCs in vivo.

One of the candidate sequences identified was an upstream sequence of the Neurofilament heavy gene (Nefh). Prior to the present study, promoter sequence for this gene had not been characterised. As described in the examples, an approximately 2.5 kb upstream fragment of the murine Nefh gene was shown to efficiently direct expression preferentially to RGCs when administered intravitreally to adult wild type mice using AAV2, in contrast to the broad expression pattern observed with the CMV promoter. Moreover, the inventors have further identified sequence regions within the murine, human and other mammalian Nefh promoter areas that are conserved between species, indicating their importance to its promoter function and moreover have demonstrated that such a promoter sequence can advantageously be used in AAV-mediated ocular gene delivery.

Accordingly, in a first aspect of the invention, there is provided method of treatment of ocular disease, wherein said method comprises administering to an eye an isolated nucleic acid molecule having promoter activity, wherein said nucleic acid molecule comprises at least Neurofilament heavy gene promoter conserved region A and optionally one or more of Neurofilament heavy gene promoter conserved regions D, F, D1, K, B, C and E;

wherein Neurofilament heavy gene promoter conserved region A is a nucleotide sequence having the nucleotide sequence shown as SEQ ID NO: 1, or a functional variant thereof; Neurofilament heavy gene promoter conserved region D is a nucleotide sequence having the nucleotide sequence shown as SEQ ID NO: 2, or a functional variant thereof; Neurofilament heavy gene promoter conserved region F is a nucleotide sequence having the nucleotide sequence shown as SEQ ID NO: 3, or a functional variant thereof; Neurofilament heavy gene promoter conserved region D1 is a nucleotide sequence having the nucleotide sequence shown as SEQ ID NO: 4, or a functional variant thereof; Neurofilament heavy gene promoter conserved region K is a nucleotide sequence having the nucleotide sequence shown as SEQ ID NO: 5, or a functional variant thereof; Neurofilament heavy gene promoter conserved region B is a nucleotide sequence having the nucleotide sequence shown as SEQ ID NO: 6, or a functional variant thereof; Neurofilament heavy gene promoter conserved region C is a nucleotide sequence having the nucleotide sequence shown as SEQ ID NO: 7, or a functional variant thereof; and Neurofilament heavy gene promoter conserved region E is a nucleotide sequence having the nucleotide sequence shown as SEQ ID NO: 8, or a functional variant thereof.

A second aspect of the invention, provides an isolated nucleic acid molecule having promoter activity for use in the treatment of ocular disease, wherein said nucleic acid molecule comprises at least Neurofilament heavy gene promoter conserved region A; and optionally one or more of Neurofilament heavy gene promoter conserved regions D, F, D1, K, B, C and E;

wherein Neurofilament heavy gene promoter conserved region A is a nucleotide sequence having the nucleotide sequence shown as SEQ ID NO: 1, or a functional variant thereof; Neurofilament heavy gene promoter conserved region D is a nucleotide sequence having the nucleotide sequence shown as SEQ ID NO: 2, or a functional variant thereof; Neurofilament heavy gene promoter conserved region F is a nucleotide sequence having the nucleotide sequence shown as SEQ ID NO: 3, or a functional variant thereof; Neurofilament heavy gene promoter conserved region D1 is a nucleotide sequence having the nucleotide sequence shown as SEQ ID NO: 4, or a functional variant thereof; Neurofilament heavy gene promoter conserved region K is a nucleotide sequence having the nucleotide sequence shown as SEQ ID NO: 5, or a functional variant thereof; Neurofilament heavy gene promoter conserved region B is a nucleotide sequence having the nucleotide sequence shown as SEQ ID NO: 6, or a functional variant thereof; Neurofilament heavy gene promoter conserved region C is a nucleotide sequence having the nucleotide sequence shown as SEQ ID NO: 7, or a functional variant thereof; and Neurofilament heavy gene promoter conserved region E is a nucleotide sequence having the nucleotide sequence shown as SEQ ID NO: 8, or a functional variant thereof.

A third aspect provides an isolated nucleic acid molecule having promoter activity, wherein said nucleic acid molecule comprises Neurofilament heavy gene promoter conserved region A and optionally one or more of Neurofilament heavy gene promoter conserved regions D, F, D1, K, B, C and E, wherein said nucleic acid molecule comprises no more than three of the group of Neurofilament heavy gene promoter conserved regions consisting of Neurofilament heavy gene promoter conserved regions D, F, D1, and K and no more than four of the group of Neurofilament heavy gene promoter conserved regions consisting of Neurofilament heavy gene promoter conserved regions D, F, B, C, and E;

wherein Neurofilament heavy gene promoter conserved region A is a nucleotide sequence having the nucleotide sequence shown as SEQ ID NO: 1, or a functional variant thereof; Neurofilament heavy gene promoter conserved region D is a nucleotide sequence having the nucleotide sequence shown as SEQ ID NO: 2, or a functional variant thereof; Neurofilament heavy gene promoter conserved region F is a nucleotide sequence having the nucleotide sequence shown as SEQ ID NO: 3, or a functional variant thereof; Neurofilament heavy gene promoter conserved region D1 is a nucleotide sequence having the nucleotide sequence shown as SEQ ID NO: 4, or a functional variant thereof; Neurofilament heavy gene promoter conserved region K is a nucleotide sequence having the nucleotide sequence shown as SEQ ID NO: 5, or a functional variant thereof; Neurofilament heavy gene promoter conserved region B is a nucleotide sequence having the nucleotide sequence shown as SEQ ID NO: 6, or a functional variant thereof; Neurofilament heavy gene promoter conserved region C is a nucleotide sequence having the nucleotide sequence shown as SEQ ID NO: 7, or a functional variant thereof; and Neurofilament heavy gene promoter conserved region E is a nucleotide sequence having the nucleotide sequence shown as SEQ ID NO: 8, or a functional variant thereof.

In one embodiment of the first, second, or third aspect of the invention, said nucleic acid molecule comprises Neurofilament heavy gene promoter conserved region A and Neurofilament heavy gene promoter conserved region D, or Neurofilament heavy gene promoter conserved region A and Neurofilament heavy gene promoter conserved region F. In one embodiment of the first, second, or third aspect of the invention, said nucleic acid molecule comprises each of Neurofilament heavy gene promoter conserved region A, Neurofilament heavy gene promoter conserved region D, and Neurofilament heavy gene promoter conserved region F.

In the context of the present application, the term “Neurofilament heavy gene promoter conserved region(s)” may be abbreviated to “NEFH promoter conserved region” or “Nefh promoter conserved region”. Unless the context demands otherwise, the terms should be considered interchangeable, with neither NEFH promoter nor Nefh promoter implying species specificity. Thus reference to a “NEFH promoter conserved region” should not be considered to be limited to a human Neurofilament heavy gene promoter conserved region but may encompass a corresponding murine Neurofilament heavy gene promoter conserved region or indeed a corresponding Neurofilament heavy gene promoter conserved region of another species. Likewise, unless the context demands otherwise, reference to a “Nefh promoter conserved region” should not be considered to be limited to a murine Neurofilament heavy gene promoter conserved region but may encompass a corresponding human Neurofilament heavy gene promoter conserved region or indeed a corresponding Neurofilament heavy gene promoter conserved region of another species.

In the context of the present invention, NEFH promoter conserved region A is a nucleotide sequence having the nucleotide sequence shown as SEQ ID NO: 1, or a functional variant thereof; NEFH promoter conserved region D is a nucleotide sequence having the nucleotide sequence shown as SEQ ID NO: 2, or a functional variant thereof; and NEFH promoter conserved region F is a nucleotide sequence having the nucleotide sequence shown as SEQ ID NO: 3, or a functional variant thereof.

| | |
|---|---|
| | SEQ ID NO: 1 |
| | CCCTGCCCCGCCCCTCTCACTGCGGCGGAGCCGGT |
| | CGGCCGGGGGGCCGCAGGGGAGGAGGCGGAGAGGG |
| | CGGGGCCCTCCTCCCCACCCTCTCACTGCCAAGGG |
| | GTTGGACCCGGCCGCGGCGGCTATAAAAGGGCCGG |
| | CGCCCTGGTG |
| | SEQ ID NO: 2 |
| | GGAAAAACAAGGGTGGGAGGACACAGCTTGTCCAA |
| | GGTCATTC |
| | SEQ ID NO: 3 |
| 3 | TGCTGTCAGCTGCTTGTGAGCCTTCTCACATCCAG |
| | AGAATGTATCAGCATTGTGCAGACTGAAAAGACCC |
| | AGAGGAACAAGGCTCCAATGGCAAAATTCCAAGTA |
| | GAATGACAAATAAATGGGGAGCCATCTGAGAGCAA |
| | GGGAGTCCTGCCCAACACCCGCCCCATGCCTTTCT |
| | CAGGGACCTCAGACCAGCCACTCACCTCCATCCTC |
| | CCAGCACCACCTGCAACCAGCCCCTTGCCCTCTGC |
| | AAACTGGAGCACGACTGGATCTTTAGATGGGGGAA |
| | AAATGCTTCATCATGTTCTGCTGCTTCATGCAAAA |
| | CCAGAAACTCCCTCCCCCTCTTCCCTCCTCCCAGC |
| | GCACTCTCCT |

In addition, further regions of the promoter have been identified as showing high levels of conservation across placental mammals. For example, the inventors have shown that in many placental mammals, including humans and other primates, the regions identified herein as D1 and K are also highly conserved.

Optionally, the isolated nucleic acid of the first, second or third aspect of the invention further comprises at least one of the conserved regions selected from: NEFH promoter conserved region D1, and K. In one embodiment, said nucleic acid molecule comprises each of NEFH promoter conserved regions D1 and NEFH promoter conserved region K. In an embodiment of the first or second aspect of the invention, said nucleic acid molecule comprises each of NEFH promoter conserved regions A, D, D1, K, and F.

In the context of the present invention, NEFH promoter conserved region D1 is a nucleotide sequence having the nucleotide sequence shown as SEQ ID NO: 4, or a functional variant thereof; and NEFH promoter conserved region K is a nucleotide sequence having the nucleotide sequence shown as SEQ ID NO: 5, or a functional variant thereof.

SEQ ID NO: 4
GACCAGCAAACTGCCTAGCTGACCCCA (SEQ ID NO: 4)
SEQ ID NO: 5
GGCCCTGTCCCCGGTGCTGAAGCGCCAG (SEQ ID NO: 5)

The inventors have also shown that in mice and many other non-primate placental mammals, the regions identified herein as B, C and E are also highly conserved.

Thus, optionally, the isolated nucleic acid molecule of the first, second or third aspect of the invention further comprises at least one of the conserved murine regions selected from: Nefh promoter conserved region B, Nefh promoter conserved region C, and Nefh promoter conserved region E. For example, said nucleic acid molecule may comprise at least two of Nefh promoter conserved region B, Nefh promoter conserved region C, and Nefh promoter conserved region E, for example, Nefh promoter conserved region B and Nefh promoter conserved region C, Nefh promoter conserved region B and Nefh promoter conserved region E, or Nefh promoter conserved region C and Nefh promoter conserved region E. In one embodiment, said nucleic acid molecule comprises each of Nefh promoter conserved regions B, Nefh promoter conserved region C, and Nefh promoter conserved region E.

In a particular embodiment of the first or second aspect of the invention, said nucleic acid molecule comprises each of Nefh promoter conserved regions A, D, F, D1, K, B, C, and E.

In the context of the present invention, Nefh promoter conserved region B is a nucleotide sequence having the nucleotide sequence shown as SEQ ID NO: 6, or a functional variant thereof; Nefh promoter conserved region C is a nucleotide sequence having the nucleotide sequence shown as SEQ ID NO: 7, or a functional variant thereof; and Nefh promoter conserved region E is a nucleotide sequence having the nucleotide sequence shown as SEQ ID NO: 8, or a functional variant thereof.

SEQ ID NO: 6
SEQ ID NO: 6
TAACT
SEQ ID NO: 7
SEQ ID NO: 7
CTTTCAAACTCCA
SEQ ID NO: 8
SEQ ID NO: 8
TTAACCCTTCCCATCCGAGGAGCGGCTGCTGTCCGTGGTGCTGA
AGCGATAGCGGCACGGGCGGCTCCGTCCAC

As described in the examples, the inventors have shown that the upstream promoter region including Nefh promoter conserved regions A, B, C, D, E, and F in the mouse and NEFH promoter conserved regions A, D1, D, F, and K in the human sequence are comprised within the 2500 to −1 upstream sequence and that utilising a promoter sequence comprising this portion of the upstream region of the gene confers cell-specificity on expression. Accordingly, in one embodiment, the isolated nucleic acid molecule of the first or second aspect of the invention comprises 2501 bp of human NEFH upstream nucleic acid sequence shown as SEQ ID NO:21. Optionally, the isolated nucleic acid molecule of the first, second or third aspect comprises less than 3000 base pairs, for example less than 2700 base pairs, such as less than 2500 base pairs, for example less than 2300, such as less than 2000, such as less than 1500, such as less than 1000 base pairs. In one embodiment of the first or second aspects of the invention, the isolated nucleic acid molecule of the first aspect of the invention consists of the nucleic acid sequence shown as SEQ ID NO: 21.

SEQ ID NO: 21
CAGAATACATCTTGGAGCCCCCTTTTTACCCCAAACCCCCATTCCTCCTT
GCTGTCAGCTGCTTGTGAGCCTTCTCACATCCAGAGAATGTATCAGCATT
GTGCAGACTGAAAAGACCCAGAGGAACAAGGCTCCAATGGCAAAATTCCA
AGTAGAATGACAAATAAATGGGGAGCCATCTGAGAGCAAGGGAGTCCTGC
CCAACACCCGCCCCATGCCTTTCTCAGGGACCTCAGACCAGCCACTCACC
TCCATCCTCCCAGCACCACCTGCAACCAGCCCCTTGCCCTCTGCAAACTG
GAGCACGACTGGATCTTTAGATGGGGGAAAAATGCTTCATCATGTTCTGC
TGCTTCATGCAAAACCAGAAACTCCCTCCCCCTCTTCCCTCCTCCCAGCG
CACTCTCCTTCCAGTAAAAAGTGGTTAAAGGGACAGCGCCATCAATTTCC
CAGCTCTGAGGGTCTGCTTAGAACTAGGGGGCTGGAAGGAGACAGAGGGC
AAAGAGAAAGGAACTGGCAGAGGTCTTTCCTGGGGGATATGTCTGTTCTG
TCCTGGGGATCCTGGAGCAGGAAAACCCGCGTAAAGTAGGGGTGTAGTGG
GTGTTGAGATAACTGCCTGGGGGAGGTTCAGAGTGGAAGTACGAGTCTAC
AAACTCTCAAGGGCGTCTCAGGGCTCCCAGCATCCCCAGGGGTCCTTTCG
CAGGGGTCCCTAAGCAGGAGGGGAACAGCCCAGAAAACACGGAACTGGAC
CCCCGACAGGAAGTCCAGGGAGGGGTCCCTGGCTCACTATGTGACCCTGC
TGGATCACTTGCCTCCCCTCTCGGGTCCCCTCAGCACAGTGTCCCTCCCT
TCCTTCCCCTAAAGTAAAAGCAGAGGGTTAATCTCTTTCCCCGCCCCACG
CCCAACAAAGAGCAGGCCCTGTCCCCGGTGCTGAAGCGCCAGCCGCAGCA
CCACCCCCACTCCCACAGCATAAAACATGAGCCAAAACCAATAAAGAGCC
AAATGTCACAGCCGTTGCAGGGCCCCCTAAATCCTGGGGACCCCTTCTTC
TACCTGACATCCTATTGGGGTGAGGGACTTTGGTACTCAGAAAGCATCTC
ATCACTTCCCTGTAAGAGAGAAGGGATGCCGACTCAGGCGCCTGCTTGTC
TGTTACAGGAGTGGGGGAAGAGAGGACAAGTTGAGGCTGAGAAGATGGGG
AGGGGGAGGGAGAAAAGAGGACTTCCTAGTGTTGACAGAACGGCAAGATG
TGGGTTCCCCATCCCCAGTTCAGCCAGAGACCCCTCAAAGTGGAACTTCC
TGGGGCAGTCGGGGGTCAGGAGTTGGAGCTTGTCTCTGGGGCAAGACCCC
TTCGTTGTACAGATGGAAAAACAAGGGTGGGAGGACACAGCTTGTCCAAG
GTCATTCGACCAGCAAACTGCCTAGCTGACCCCAGTGTGCAGAAGCTGGC
TCGGGTGACACCCATCATTTCCCCCCACCCCACACAGGGGCCAGCTCTCT
CAACTTCATGCCCAAGCCCTCCTACGGTACCCCCACTGTAGGTTCTCTGC
CCCTCAAACTCAGCCCAGCTTTCTCCTGCCTGTTCAGGGGACCTTCTGCC
CGCTTCGCTGAGGGTCCGTCCCCTTTACTGGGGCTGGCAGCAGGGTCTCC
CATCTCCTCTCTCGGGGGCCACTGCAGACTTTTTAGAGAACGCCTTGCCT
CCCCCCAACCCCACCCATCCGGGGTTCCCTCTCTCCATCCTCTGCAGTGT
CTCCCATACCCCCATTCAGGGTAGCCTTGCTATTCTCCCCAACTCCAGGT
CCCCCTTCATCTATTCCGGGGCTGGCCGCGGAGTTTCCTGAGCGCTCTCC
AAGTGGGTCCTCTAGATGTTAGGAGAACACTGTACCTCCCCCGGTCAGGG
GTCTCCTGTCTCCGTTCTATGGAGCGTCCATGCTCCCATTCAGGACTGCC
TTGCTCCCTCCTCTGTTCCGGGGCTGGCTGCACAGTCTCTGCACCCCCTA
TCCTGAAAGCCTCTCTTAACTATTTGGAAAGCCTCGTGTCCTGTCTCATA
CAGGGATCCCCTCATCCTAATGACTGCAATCTTCCATTGCTCCATCCCGA
GGGCATCCTGCCCCTATTCCCATCAGGTTTCTCCTTGTCCTCTCCCTGTT
TCAAGTCCCCTTTCTTATTCCGAACACACTCGCAGGCTCTTCCGACGCGC
ACCCGGGGGTCCTCACTGGCCCACTCCGGGAGTCCTCTGCCCGCTTCCCC
GACCTCGAGGGTCTCCTCTGACGCAGCGTCGATTCCCCTTCCCTCCTCGG
TCCCCTGCCCCGCCCCTCTCACTGCGGCGGAGCCGGTCGGCCGGGGGGCC
GCAGGGGAGGAGGCGGAGAGGGCGGGGCCCTCCTCCCCACCCTCTCACTG
CCAAGGGGTTGGACCCGGCCGCGGCGGCTATAAAAGGGCCGGCGCCCTGG
TGCTGCCGCAGTGCCTCCCGCCCCGTCCCGGCCTCGCGCACCTGCTCAGG
C

As described in the examples, the inventors have shown that, for retinal ganglion cell specificity, the full length NEFH promoter is not required. Moreover, all of the conserved regions are not required. For example, as shown herein, promoters having only one of the recited conserved regions, conserved region A, in the absence of the other conserved regions, provides efficient targeting to retinal ganglion cells.

Accordingly, in one embodiment of the invention, the isolated nucleic acid molecule of or for use in the invention comprises fewer than three of NEFH promoter conserved regions D, F, D1, K, B, C and E. For example, in one embodiment, said isolated nucleic acid molecule comprises conserved regions A and F in the absence of NEFH promoter conserved regions D, D1, K, B, C and E. In another embodiment, said isolated nucleic acid molecule comprises conserved region A in the absence of NEFH promoter conserved regions D, D1, K, B, C and E.

Where the isolated nucleic acid molecule of or for use in the invention comprises two or more of conserved regions A, D, F, D1 and K, the isolated nucleic acid may comprise between two recited conserved regions a spacer sequence of a length in the range 20-180%, for example 50-150%, such as 70-130%, for example 80-120%, such as 90-110%, such as 95-100% of the sequence separating said recited conserved regions in the nucleic acid sequence shown as SEQ ID NO: 21. In one such embodiment, the isolated nucleic acid molecule may comprise between each recited conserved region and its adjacent recited conserved region in said isolated nucleic acid molecule a spacer sequence of a length in the range 20-180%, for example 50-150%, such as 70-130%, for example 80-120%, such as 90-110%, such as 95-100% of the sequence separating said recited conserved regions in the nucleic acid sequence shown as SEQ ID NO: 21. In an alternative embodiment, where the isolated nucleic acid molecule of or for use in the invention comprises two or more of conserved regions A, D, F, D1 and K, the isolated nucleic acid molecule may comprise between two recited conserved regions a spacer sequence of a length in the range 20-180%, for example 50-150%, such as 70-130%, for example 80-120%, such as 90-110%, such as 95-100% of the sequence separating one of said recited conserved regions from one of its adjacent conserved regions in the nucleic acid sequence shown as SEQ ID NO: 21.

In some embodiments, the spacer between a recited conserved region and its adjacent recited conserved region has at least 90% homology, for example at least 95%, 98% or 100% homology to the corresponding spacer sequence separating said conserved region and its adjacent recited conserved region in the nucleic acid sequence shown as SEQ ID NO: 21

In an embodiment of the first or the second aspect of the invention, the isolated nucleic acid molecule the isolated nucleic acid molecule comprises or consists of the nucleic acid sequence shown as SEQ ID NO: 21.

As described herein, in some embodiments of the invention, the isolated nucleic acid molecule comprises at least one of the conserved regions selected from: Nefh promoter conserved region B, Nefh promoter conserved region C, and Nefh promoter conserved region E. In one embodiment of the first or second aspects of the invention, the isolated nucleic acid molecule comprises each of Nefh promoter conserved regions A, D, F, B, C, and E. In one such embodiment, the isolated nucleic acid molecule comprises each of Nefh promoter conserved regions A, D, F, B, C, and E having the nucleic acid sequences shown as SEQ ID NOS: 9, 12, 15, 6, 7, and 8 respectively. In another embodiment, the isolated nucleic acid molecule may comprise each of Nefh promoter conserved regions A, D, F, B, C, and E having the nucleic acid sequences shown as SEQ ID NOS: 10, 13, 16, 18, 19, and 20 respectively.

Where the isolated nucleic acid molecule of or for use in the invention comprises two or more of conserved regions A, D, F, B, C, and E, the isolated nucleic acid molecule may comprise between two recited conserved regions a spacer sequence of a length in the range 20-180%, for example 50-150%, such as 70-130%, for example 80-120%, such as 90-110%, such as 95-100% of of the sequence separating said recited conserved regions in the nucleic acid sequence shown as SEQ ID NO: 22. In one such an embodiment, the isolated nucleic acid molecule may comprise between each recited conserved region and its adjacent recited conserved region in said isolated nucleic acid molecule a spacer sequence of a length in the range 70-130%, for example 80-120%, such as 90-110%, such as 95-100% of of the sequence separating said recited conserved regions in the nucleic acid sequence shown as SEQ ID NO: 22. In an alternative embodiment, the isolated nucleic acid molecule comprises between two recited conserved regions a spacer sequence of a length in the range 20-180%, for example 50-150%, such as 70-130%, for example 80-120%, such as 90-110%, such as 95-100% of of the sequence separating one of said recited conserved regions from one of its adjacent conserved regions in the nucleic acid sequence shown as SEQ ID NO: 22. In an embodiment of the first or second aspects of the invention, the isolated nucleic acid molecule may consist of the nucleic acid sequence shown as SEQ ID NO: 22.

In a fourth aspect of the invention, there is provided an expression cassette comprising the isolated nucleic acid molecule according to the third aspect of the invention and one or more heterologous polynucleotide sequences with which the nucleic acid molecule is operably linked.

As described in the examples, the promoter sequence of the invention may advantageously provide preferential gene expression to the ganglion cell layer (GCL). Accordingly, in the expression cassette of the fourth aspect of the invention, said nucleic acid molecule having promoter activity may provide preferential expression of said one or more heterologous polynucleotide sequences in the ganglion cell layer of the eye.

The promoter molecule and the expression cassette may be provided in a vector. Accordingly, in a fifth aspect of the invention there is provided a vector comprising the isolated nucleic acid of the first aspect or the expression cassette of the second aspect. Any suitable vector may be used. Vectors may be, for example, viral vectors, non-viral vectors, or naked DNA.

As described herein, the inventors have demonstrated that the promoter sequence of the invention is advantageously small enough to be used with adenoassociated viral vectors (AAVs). Accordingly in the fifth aspect of the invention, the vector may optionally be an AAV vector. Any suitable AAV vector may be used in the invention. Vectors of the invention may include additional elements other than the promoter sequence and heterologous polynucleotide sequences with which the nucleic acid molecule is operably linked. In an embodiment, the vector comprises at least one regulatory element selected from the group consisting of enhancer sequence, a stuffer, an insulator, a silencer, an intron sequence, a post translational regulatory element, a polyadenylation site, and a transcription factor binding site. The vector may comprise a sequence encoding a neurotrophic or neuroprotective factor. In another aspect the vector comprises more than one expression cassette. In another embodiment two vectors, one containing at least one of the conserved murine or human sequences from the Nefh upstream region and a second containing a marker gene, neurotrophic or neuroprotective factor may be co-administered or administered successively.

In a sixth aspect of the invention, there is provided a cell comprising the isolated nucleic acid molecule according to the third aspect of the invention, the expression cassette according to the fourth aspect of the invention, or the vector according to the fifth aspect of the invention.

The invention is contemplated for use in therapeutic treatments, for example in gene therapy treatments for eye diseases or conditions.

Accordingly, a sixth aspect of the invention provides a therapeutic composition comprising the isolated nucleic acid molecule according to the third aspect of the invention, the expression cassette according to the fourth aspect of the invention, the vector according to the fifth aspect of the invention, or the cell according the sixth aspect of the invention.

A seventh aspect provides the isolated nucleic acid molecule according to the third aspect of the invention, the expression cassette according to the fourth aspect of the invention, the vector according to the fifth aspect of the invention, or the cell according the sixth aspect of the invention for use in medicine.

In one embodiment of the invention, the ocular disease is Leber Hereditary Optic Neuropathy (LHON). In another embodiment, the ocular disease is dominant optic atrophy (DOA). In another embodiment, the ocular disease is glaucoma. In another embodiment, the ocular disease involves an optic neuropathy. In another embodiment, the disease may be syndromic with a RGC layer and or optic nerve involvement. RGCs connect to the optic nerve and thus RGC death commonly results in optic nerve deterioration. Accordingly, directing treatment to the RGCs or delivering an entity to the RGCs to benefit the optic nerve is considered a valuable approach in treatment of diseases affecting the optic nerve.

In another aspect, the invention provides transgenic animals comprising the isolated nucleic acid molecule according to the third aspect of the invention, the expression cassette according to the fourth aspect of the invention, the vector according to the fifth aspect of the invention, or the cell according the sixth aspect of the invention.

In another aspect of the invention, there is provided a kit for the identification of RGCs, wherein the kit comprises the isolated nucleic acid molecule according to the third aspect of the invention, the expression cassette according to the fourth aspect of the invention, the vector according to the fifth aspect of the invention.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 illustrates schematically putative promoter identification methodology. Transcriptomic data (Kim et al. 2006) was used to identify candidate genes, based on expression levels in the retina (EL_{OR}) and the GCL (EL_{GCL}). Enrichment factor (EF) for the GCL was calculated as EF=EL_{GCL}/EL_{OR}; top candidates were identified based on EF. A gene score (GS) was calculated as a means of discerning between candidates.

FIG. 2A illustrates analysis of 5′ upstream sequence of five candidate promoter sequences. Regions ~2.5 kb upstream of the transcriptional start were analysed. The genes displayed represent the most highly expressed genes of the genes that are enriched >10-fold in RGCs. The y-axis represents conservation across mammals (CS), where 0 equals no significant conservation and 1 equals full conservation across mammalian species in the UCSC genome database.

FIG. 2B is a schematic illustration of regions of significant sequence conservation in the putative Nefh promoter region in both human and mouse. Sequence conservation within the 2.5 kb region directly upstream of the Nefh transcriptional start site, the putative Nefh promoter region. Units of conservation have been defined as blocks (A-F, D1 and K).

FIG. 3A illustrates diagrammatic representations of constructs used in the examples. EGFP-expressing constructs have been cloned and AAV generated (AAV. Nefh-EGFP and AAV.CMV-EGFP). A third construct expressing an I82VNdi1 gene has been made and AAV generated (AAV.Nefh-182VNdi1).

FIG. 3B Diagrammatic representation of constructs used in the examples. EGFP-expressing constructs have been cloned and AAV generated (AAV. Nefh-EGFP (either human or mouse NEFH promoter sequence) and AAV.A-EGFP and AAV.A+F-EGFP, AAV.A-spacer-F-EGFP.

FIGS. 4A-4F illustrate analysis of Nefh mediated EGFP expression in vivo. Retinas were injected intravitreally with AAV.CMV-EGFP (3×10⁹ vg; FIG. 4A and FIG. 4D) or two different doses of AAV.Nefh-EGFP (3×10⁹ vg; FIG. 4B and FIG. 4E; and 9×10⁹ vg; FIG. 4C and FIG. 4F). Transduced eyes (n=4-5) were fixed and cryosectioned 12 weeks post-delivery. FITC-labeled immunocytochemistry was performed for EGFP. DAPI was used for nuclear counterstaining; DAPI signals are overlaid on the right side of the images. ONL: outer nuclear layer; INL: inner nuclear layer, GCL: ganglion cell layer. Arrowheads: transduced cells in the INL. Scale bars: 500 μm (C) and 25 μm (F).

FIGS. 5A-5O illustrate the results of immunocytochemistry of AAV.Nefh-EGFP transduced retinas. Eyes were injected intravitreally with AAV.Nefh-EGFP (3×10⁹ vg). Transduced eyes (n=5) were fixed and cryosectioned 12 weeks post-delivery. Immunocytochemistry was performed for Brn3a (Cy3; FIGS. 5A-5E), ChAT (Cy3; FIGS. 5F-FJ) and GABA (Cy3; FIGS. 5K-5O) in combination with EGFP labeling (FITC). DAPI was used for nuclear counterstaining. Rectangles (in FIG. 5B, FIG. 5G and FIG. 5L) indicate positions of the enlarged areas. FIG. 5A, FIG. 5F and FIG. 5K: Cy3 label; FIG. 5B, FIG. 5G and FIG. 5L: Cy3, FITC and DAPI overlaid. FIG. 5C, FIG. 5H and FIG. 5M: FITC label; FIG. 5D, FIG. 5I and FIG. 5N: Cy3 label; FIG. 5E, FIG. 5J and FIG. 5O: Cy3, FITC and DAPI labels overlaid. FIGS. 5C-5E: Bold arrowheads: transduced Brn3a-positive cells. Regular arrowheads: un-transduced Brn3a-negative cells. Double arrowhead: a transduced Brn3a-negative cell. FIGS. 5H-5J: Bold arrowheads: transduced ChAT-negative cells. Regular arrowhead: a transduced ChAT-positive cell. FIGS. 5M-5O: Bold arrowheads: transduced GABA-negative cells. Regular arrowheads: un-transduced GABA-positive cells. Double arrowhead: an un-transduced GABA-positive cell. ONL: outer nuclear layer; INL: inner nuclear layer, GCL: ganglion cell layer. Scale bars: 25 μm (FIG. 5F and FIG. 5H).

FIGS. 6A-6D illustrate barcharts summarising CMV or Nefh mediated EGFP expression in vivo. Retinas were injected intravitreally with AAV.CMV-EGFP (3×10⁹ vg; FIG. 6A and FIG. 6D) or two different doses of AAV.Nefh-EGFP (3×10⁹ vg; FIG. 6B and FIG. 6E; and 9×10⁹ vg; FIG. 6C and FIG. 6F). Transduced eyes (n=4-5) were fixed and cryosectioned 12 weeks post-delivery. Immunocytochemistry was performed for Brn3a, ChAT, GABA and EGFP; DAPI was used for nuclear counterstaining. Manual quantification of labeled and co-labeled cells was performed in the immunolabelled retinal sections. FIG. 6A: Distribution of EGFP positive cells was determined in the ganglion cell (GCL) and the inner nuclear layers (INL). Additionally, co-localisation of EGFP with Brn3a (FIG. 6B), ChAT (FIG. 6C) and GABA (FIG. 6D) was determined in the GCL. ***: p<0.001; *p<0.05 (ANOVA).

FIGS. 7A-7H illustrate flow cytometry analysis of Nefh mediated EGFP expression in vivo. Eyes were injected intravitreally with either AAV.CMV-EGFP (3×10⁹) or AAV.Nefh-EGFP (9×10⁹ vg). Three weeks post-injection, retinas were dissociated and processed for flow cytometry analysis, using a Thy1 antibody conjugated to PE-Cy5. Nucleated cell populations were identified on the basis of DRAQ5 positive labelling (data not shown) and forward (FSC) and side (SSC) scatter (FIG. 7A), and singlets identified (FIG. 7B, FIG. 7C). Thy1 (x-axis) and EGFP (y-axis) gates were created based on wildtype retinas that had not been treated with Thy1 antibody and wildtype retinas that had been treated with Thy1 antibody, representing Thy1-negative (FIG. 7D) and Thy1-positive (FIG. 7E) control samples. Using these pre-defined gates transduced retinal samples (n=6 per group) were sorted against EGFP and PE-Cy5 (Thy1; FIG. 7F, FIG. 7G). Percentages of cells in each quadrant are indicated. Enrichment values were generated by dividing the percentage of Thy1 and EGFP double positive cells by the percentage of EGFP-positive Thy1-negative cells. Thy1 positive cells (from n=12 retinal samples) and non-labeled singlets with a similar FFC/SSC profile (from n=9 retinal samples) were collected and pooled and Thy1 mRNA levels were established by RT-QPCR (FIG. 7H). Thy1 mRNA enrichment in Thy1 antibody positive cells was calculated from the ΔCt value divided by the ratio of Thy1-positive cells to whole retinal cells.

FIG. 8 illustrates analysis of Nefh specificity in vivo. Retinas injected subretinally (e-h) with AAV-Nefh-EGFP were sectioned and imaged using a Zeiss Axioplan fluorescent microscope. The left column displays the whole retinal section, while the right shows higher magnification examples. Abbreviations: ONL, outer nuclear layer. INL, inner nuclear layer. GCL, ganglion cell layer.

FIGS. 9A-9B. illustrate the results of mRNA analysis and immunocytochemistry of Nefh mediated EGFP expression in vivo. Retinas were injected intravitreally with 6.6×10⁸ vg of AAV.NEFH-EGFP (SEQ ID NO: 128) and AAV.Nefh-EGFP (SEQ ID NO: 129) and EGFP expression analysed three weeks post injection. FIG. 9A. [N1] RT-qPCR (n=5-6) B-Actin was used as an internal control. Relative expression levels are given as a percentage of EGFP expression from AAV.Nefh-EGFP. Expression levels from AAV.Nefh-EGFP and AAV.NEFH-EGFP were 100%±39.2% and 101%±56.4%. EGFP expression levels were not significantly different. FIG. 9B Three weeks post-injection of AAV vectors eyes were harvested and fixed in 4% pfa in PBS o/n. 12 μm retinal cryosections were immunostained for EGFP using Cy5 conjugated secondary antibody; DAPI was used for nuclear counterstain. A and C: NEFH; B and D: Nefh. Scale bar in D indicates 50 μm.

FIGS. 10A-10B illustrate the results of mRNA analysis and immunocytochemistry of EGFP expression from AAV.Nefh-EGFP, AAV.A-EGFP, AAV.A+F-EGFP and AAV.A-spacer-F-EGFP (SEQ ID NOs 129, 124, 126, 125). FIG. 10A, Relative levels were compared to levels expressed from AAV.Nefh-EGFP where EGFP was driven from a 2.2 kb murine Nefh promoter. 6.6×10⁸ vg of AAV.Nefh-EGFP (n=5) and AAV.A-EGFP (n=6) and AAV.A+F-EGFP (n=6) and AAV.A-spacer-F-EGFP (n=6) vector were injected intravitreally into wild type 129 mice. 4 weeks post-injection total RNA was isolated from whole retinas taken from injected mice. Levels of EGFP RNA expression were determined by RT QPCR standardised to housekeeping gene B-actin. Y-axis represents relative percentage EGFP expression. Levels of EGFP RNA expression from AAV.Nefh-EGFP was considered to be 100%. Relative levels of EGFP RNA expression from AAV.Nefh-EGFP, AAV.A-EGFP, AAV.A+F-EGFP and AAV.A-spacer-F-EGFP were 100%±45.1%, 33.8%±14.6%, 20.4%±20.5% and 5.4%±4.80% respectively. EGFP expression levels from AAV.Nefh-EGFP was significantly higher than from any of the other constructs (p≤0.05). In addition, levels of EGFP RNA expression from AAV.A-EGFP were significantly higher than from AAV.A-spacer-F-EGFP (p≤0.05). FIG. 10B. Four weeks post-injection of AAV vectors eyes were harvested and fixed in 4% pfa in PBS o/n. 12 μm retinal cryosections were immunostained for EGFP using Cy5 conjugated secondary antibody; DAPI was used four nuclear counterstain. A, E, I: Nefh-EGFP B, F, J: A-EGFP; C, G, K: A+F-EGFP; D, H, L: A-spacer-F-EGFP. The part images in the rectangles in E, F, G and H are enlarged in I, J, K and L respectively. Cy5 exposure times were 3× longer for A+F (C, G and K) and A-spacer-F (D, H and L). Scale bars in H and L indicate 50 μm.

FIGS. 11A-11F illustrate in tabulated form the sequence of the AAV.A-EGFP, AAV.A-spacer-F-EGFP, AAV.A+F-EGFP, AAV.CMV-EGFP, huNEFH-EGFP and AAV.muNefh-EGFP constructs used in the examples.

### DETAILED DESCRIPTION OF THE INVENTION

The invention relates to use of conserved sequences from the upstream sequence of the Nefh gene to enhance expression of genes. In a particular aspect the invention relates to use of such conserved sequences to enhance expression of genes from adeno associated virus (AAV) vectors.

Specifically, the invention utilises the nucleic acid molecule comprising at least one of the conserved regions selected from: Nefh promoter conserved region A; Nefh promoter conserved region D, and Nefh promoter conserved region F.

Additionally, the isolated nucleic acid molecule of the first aspect of the invention optionally may further comprise at least one of the conserved regions selected from: NEFH promoter conserved region D1, and NEFH promoter conserved region K.

Furthermore, the isolated nucleic acid molecule of the first aspect of the invention optionally may further comprise at least one of the conserved regions selected from: Nefh promoter conserved region B, Nefh promoter conserved region C, and Nefh promoter conserved region E.

NEFH promoter conserved region A is a nucleotide sequence having the nucleotide sequence shown as SEQ ID NO: 1, or a functional variant thereof; NEFH promoter conserved region D is a nucleotide sequence having the nucleotide sequence shown as SEQ ID NO: 2, or a functional variant thereof; and NEFH promoter conserved region F is a nucleotide sequence having the nucleotide sequence shown as SEQ ID NO: 3, or a functional variant thereof. NEFH promoter conserved region D1 is a nucleotide sequence having the nucleotide sequence shown as SEQ ID NO: 4, or a functional variant thereof; NEFH promoter conserved region K is a nucleotide sequence having the nucleotide sequence shown as SEQ ID NO: 5, or a functional variant thereof. Nefh promoter conserved region B is a nucleotide sequence having the nucleotide sequence shown as SEQ ID NO: 6, or a functional variant thereof; Nefh promoter conserved region C is a nucleotide sequence having the nucleotide sequence shown as SEQ ID NO: 7, or a functional variant thereof; and Nefh promoter conserved region E is a nucleotide sequence having the nucleotide sequence shown as SEQ ID NO: 8, or a functional variant thereof.

In the context of the present invention, a “functional variant” includes any variant nucleic acid or that may have one or more nucleic acid substitutions but that does not have a materially different function than, or that can still hybridize under stringent hybridization conditions (0.2×SSC, 0.1% SDS) to, or that shares at least 60% identity, for example at least 65% identity, such as at least 70% identity, for example at least 80% identity, such as at least 90% identity or at least 95% sequence identity with the nucleotide sequence or nucleic acid indicated. A functional variant preferably retains at least 10%, for example 20%, 35%, 50%, 70%, 80%, 90% or greater of the functional activity of the sequence indicated. Thus, for example, where said sequence is a promoter said functional activity is promoter activity.

Particular examples of Nefh promoter conserved region A which are functional variants of the human nucleotide sequence shown as SEQ ID NO: 1 include the nucleotide sequences shown as murine SEQ ID NO:9, murine SEQ ID NO: 10, and rhesus macaque SEQ ID NO:11.

SEQ ID NO: 9
SEQ ID NO: 9
CCAGCCCCGCCCCTCTCACTGCGGAGAAGCCGGTCGGCCCGGGGCC
GCGGGGGAGGAGGTGGAGAGGGTGGGGCCCTCCTCCCCAGCCCCCC
ACTGCCGAGGGGCCGGACCGGGCCACCGCGGATATAAAAGAGCCGG
AGTCCCAGAGCTGCCGCAGTGCTGCCTGCCCCGTCCCAGCCCCGCA
CTCCCGCTC
SEQ ID NO: 10
SEQ ID NO: 10
CCCAGCCCCGCCCCTCTCACTGCGGAGAAGCCGGTCGGCCCGGGGC
CGCGGGGGAGGAGGTGGAGAGGGTGGGGCCCTCCTCCCCAGCCCCC
CACTGCCGAGGGGCCGGACCGGGCCACCGCGGATATAAAAGAGCCG
GAGTCCCAGAGCTGCCGCAGTGCTGCCTGCCCCGTCCCAGCCCCGC
ACTCCCGCTC
SEQ ID NO: 11
SEQ ID NO: 11
CCCTACCCCGCCCCTCTCACTGCGGCTGAGCCGGTCAGCCGGGGGC
CGCAGGGGAGGAGGCGGAGAGGGCGGGGCCCTCCTCCCCACCCCCT
CACTGACAAGGGGTTGGACCCGGCCGCGGCGGCTATAAAAGGGCCG
GCGCCCTGGTGCTGCCGCAGTGCCTCCAGCCCCGTCCCGGCCCCGC
GCACCTGCTC

Particular examples of human NEFH promoter conserved region D which are functional variants of the nucleotide sequence shown as SEQ ID NO: 2 include the nucleotide sequences shown as murine SEQ ID NO:12, murine SEQ ID NO: 13, and rhesus macaque SEQ ID NO:14.

SEQ ID NO: 12
SEQ ID NO: 12
GGAAAACCAAACATAGGAGAACACAATTTGTACAAGGTCATTC
SEQ ID NO: 13
SEQ ID NO: 13
TGCATCTTGTCTCTTGCACACAAGGGAAAACCAAACATAGGAG
AACACAATTTGTACAAGGTCATTCAGCTAGCGAAGCACAGAAG
CTAACCCC
SEQ ID NO: 14
SEQ ID NO: 14
GGAAAAACAAGGGTGGGAGAATACAGCTCGTCCAAGGTCATTC

Particular examples of human NEFH promoter conserved region F which are functional variants of the nucleotide sequence shown as SEQ ID NO: 3 include the nucleotide sequences shown as murine SEQ ID NO:15, murine SEQ ID NO:16, and rhesus macaque SEQ ID NO:17.

SEQ ID NO: 15
SEQ ID NO: 15
AAATCCAAGCAGTATGGGAGATAAATGGGGAAGCCATGTGGGCGTAAGGG
GGTAGAGGTCTGCATCCCAGTCCCCTCCCCATGGCATCTGCAGTGCCTCC
CAGCCTTTCTGACCCCTGCAAAGAGCAGCATGACTGGACCTTTAAATTGG
GAAAATGCTTCATCATGTTCTGCTCCATCATGAAAAACTAGAGTCTCCTC
SEQ ID NO: 16
SEQ ID NO: 16
TGCTGTCAACTGCTTGTCAGACTTCTCACCCCCAAGAAGGGCATGTGC
ATTCTGCAGACAACTGAAGAGACTCGAAGGAACAAGAATCTAATAACAAA
AATCCAAGCAGTATGGGAGATAAATGGGGAAGCCATGTGGGCGTAAGGGG
GTAGAGGTCTGCATCCCAGTCCCCTCCCCATGGCATCTGCAGTGCCTCCC
AGCCTTTCTGACCCCTGCAAAGAGCAGCATGACTGGACCTTTAAATTGGG
AAAATGCTTCATCATGTTCTGCTCCATCATGAAAAACTAGAGTCTCCTCC
CCCTCCTCCCTAGTGCACTCTCCT
SEQ ID NO: 17
SEQ ID NO: 17
TGCTGTCAGCTGCTTGTGAGCCTTCTCACATCCAGAGAATATATCAGCAT
TCTGCAGACCGAAAAGACCCAGAGGAACAAGGCTCCAATGGCAAAATTCC
AAGTAGAATGACAAATAAATGGGGAGCCATTTGAGAGCAAGGGAGTCCTG
CCCAACACCCCCTCCCCATGCCTTTCTCAGGGACCTCAGACCAGCCACTC
ACCTCCATCCTCCCAGAACCACCTGCAACCAGCCCGTTGCCCCTTGCAAA
CTGGAGCATGACTGGATCTTTAGATGGGGGAAAAATGCTTCATCATGTTC
TGCTTCTTCATGCAAAACCAGAAACTCCCTCCCCCTCTTCCCTCCTCCCA
GCGCACTCTCCT

A particular example of murine Nefh promoter conserved region B which is a functional variant of the nucleotide sequence shown as SEQ ID NO: 6 is the nucleotide sequence shown as SEQ ID NO:18.

SEQ ID NO: 18
SEQ ID NO: 18
TCTAACT

A particular example of murine Nefh promoter conserved region C which is a functional variant of the nucleotide sequence shown as SEQ ID NO:7 is the nucleotide sequence shown as SEQ ID NO:19.

SEQ ID NO: 19
SEQ ID NO: 19
GCGCTCCCTTTCTCCGTCTGCAGTGTTCTCCTTCTCAGGGTAGCTTTGCG
GTCCTTTCAAACTCCACGCCC

A particular example of murine Nefh promoter conserved region E which is a functional variant of the nucleotide sequence shown as SEQ ID NO: 8 is the nucleotide sequence shown as SEQ ID NO:20.

SEQ ID NO: 20
SEQ ID NO: 20
ATTTAACCCTTCCCATCCGAGGAGCGGCTGCTGTCCGTGGTGCTGAAGCG
ATAGCGGCACGGGCGGCTCCGTCCACTAAC

As described above, in certain embodiments of the invention, the isolated nucleic acid molecule of the first aspect of the invention comprises each of conserved regions A, D1, D, F, and K. In one such embodiment, the isolated nucleic acid molecule comprises a full length NEFH promoter sequence such as that shown as SEQ ID NO:21.

In another embodiment, the isolated nucleic acid molecule comprises a full length Nefh promoter sequence such as that shown as SEQ ID NO:22.

SEQ ID NO: 22
SEQ ID NO: 22
TGTGCTGTCAACTGCTTGTCAGACTTCTCACCCCCAAGAAGGGCATGTGCATT
CTGCAGACAACTGAAGAGACTCGAAGGAACAAGAATCTAATAACAAAAATCCA
AGCAGTATGGGAGATAAATGGGGAAGCCATGTGGGCGTAAGGGGGTAGAGGTC
TGCATCCCAGTCCCCTCCCCATGGCATCTGCAGTGCCTCCCAGCCTTTCTGAC
CCCTGCAAAGAGCAGCATGACTGGACCTTTAAATTGGGAAAATGCTTCATCAT
GTTCTGCTCCATCATGAAAAACTAGAGTCTCCTCCCCCTCCTCCCTAGTGCAC
TCTCCTGGCCTGCAGCCAGGGGCTGGGAATGAGACACAGGACAGGAAAGGGAT
CTCTTTTAGGGAATCTATCAGTTCTCCTCCTAGGGATCCCTCCAAAAGAGAAA
ACCACAGCAAACTGGGGTGCAGTGAGGCTTGAGGTAACTGCCTGGGAGAAGTT
CTGATCTGAAGAAGTCTATACTGGTTTCCAGAGCTTGTCAGTGGGCATTGGAG
TGGGGCTCTCTCTGCTCCGGGAAGAGGTTTGCAGGGAGAAAGAACTTCACAGA
GAGCCAGGCACTGGACAGGACATGCAGGGGTGGGTCACTTACATACAACCGTA
GGTCGTTTCGAGCCCGTCATATGACTCATCCAATCCTCCCCTGTACCGCACAG
AGGGACTGCTTGGAAAAGCTATGGAACCTCCCTACTCCGTTAGGCATAGATTT
AACCCTTCCCATCCGAGGAGCGGCTGCTGTCCGTGGTGCTGAAGCGATAGCGG
CACGGGCGGCTCCGTCCACTAACACCGCTTTTGACCGGAAAACCAAACCAAGA
ACGAGCCGTATAATAAAGCAAGAGCTCCAAGTCTAAGCCCCTCCGCCGTCCCC
GCCCTTTCACCTGAAGCCTCAGTAGGGCTCATGATGGAGGTCGGTGGACTTTG
GTACTGAAAAACCACTCCACCACTTCCTCGGAGCATGAAAGGGGATGCTTACG
GCAGTACTGGTTCATCTATTCTGGAAAAGGAATGAGATGCCAAGATAAAGCAG
AAAAATCGGGCAAGGAAGGGAGAAAGACAAAGTTCTCAGGTGAGAGGAACTGG
TTACTATTCCGACTGGCAATATGTGGGTTCTCCTCCCCAAAATCAGCCAGACA
TTTCCCAAGTTCGAACCTCCTAGGGGCACATGGGAGCTTGGAGCTGCATCTTG
TCTCTTGCACACAAGGGAAAACCAAACATAGGAGAACACAATTTGTACAAGGT
CATTCAGCTAGCGAAGCACAGAAGCTAACCCCACCCTGTGGCAGAACTTGGCT
TCGGTGTTGAGGCTCTTGCTGCCTACTGAGGGACCCCCTGTTCTTCGTAGGCA
GTTTTCCTTTCCGGGCAAGAGGAGACTCCACTTTCCAGTCGTGGCCACTGGAA
TTTTTAGAGAGCACCACGTTCCTCTCACCCAGCGCTCCCTTTCTCCGTCTGCA
GTGTTCTCCTTCTCAGGGTAGCTTTGCGGTCCTTTCAAACTCCACGCCCACCC
CAACCCCAACCCCGAAGCCAGCTGTACAGTTCCTTAAGCCCCTTTGGGTGGCC
CAGGGCCGCTGTAGTATCTGGGGAACACTGCACCGCCAGCTAGAAGGTCCCCA
TTTATCATCAGTAGCATCCATCATGCAACCCCATACAGAATCCCTTCGTGGGT
GACTGCAGTCTGCACTCCTCATCTCAAGGTCCTCTCTAACTATCAGGGAACCA
ACCCTGTGCTGCTTCTCAAGTGGGGGTGTCCTCTCATAGTAATCACTGCAGTC
TCCCACTGCTTCAACCCGAAGGCGCCCTGACCCATCAGTTCTGCAATCCTCTC
CCTATTTCCAGTGCCCTCTCTTATTCTGAGGGTCTTATTCTGACTAATAGGGT
CTTCCGACATGCACCTGGAGGTCTGCACTTGTCCGCTCCGGAAGTCCTTTACT
CCTTGGTCTGACCTCGGGAGGCTCTACTGACGATGCGTCGATTCCCCTTCACT
CCTGGGTCGTCCCCCCCAGCCCCGCCCCTCTCACTGCGGAGAAGCCGGTCGGC
CCGGGGCCGCGGGGGAGGAGGTGGAGAGGGTGGGGCCCTCCTCCCCAGCCCCC
CACTGCCGAGGGGCCGGACCGGGCCACCGCGGATATAAAAGAGCCGGAGTCCC
AGAGCTGCCGCAGTGCTGCCTGCCCCGTCCCAGCCCCGCACTCCCGCTCCGCT
GGCGGCCGCACCTGCTCCGGCCATG

In another embodiment, the isolated nucleic acid molecule comprises a full length rhesus macaque Nefh promoter sequence such as that shown as SEQ ID NO:23.

SEQ ID NO: 23
SEQ ID NO: 23
CAGTCCCTCTTGGAGCCCCCTTTTTACCCCAAATCCCTAGTCCTCTTTGC
TGTCAGCTGCTTGTGAGCCTTCTCACATCCAGAGAATATATCAGCATTCT
GCAGACCGAAAAGACCCAGAGGAACAAGGCTCCAATGGCAAAATTCCAAG
TAGAATGACAAATAAATGGGGAGCCATCTGAGAGCAAGGGAGTCCTGCCC
AACACCCCCTCCCCATGCCTTTCTCAGGGACCTCAGACCAGCCACTCACC
TCCATCCTCCCAGAACCACCTGCAACCAGCCCGTTGCCCCTTGCAAACTG
GAGCATGACTGGATCTTTAGATGGGGGAAAAATGCTTCATCATGTTCTGC
TTCTTCATGCAAAACCAGAAACTCCCTCCCCCTCTTCCCTCCTCCCAGCG
CACTCTCCTTCCAGTAAAACATGGTTAAAGGGACAGCGCCATCACTTTCC
CAGCTCTGAGGGTCTGCTTAGAACCAGGGGCCTTGGAAGGAGACAGAGGG
CAAAGAGAAAGGAACTGGCAGAGGTCTTTCCTGGGGGATCTGTCTGTTCT
GTCCTGGGAATCCTGGAGCAGGAAAACTCGGGTAAAGTGGGGGTGTAGTG
GGGGTTGAGATAACCGCCTGGGGGAGATTCAGAGTGCAAGTAGGAGTCTA
CAAACTCTCAAGGGGGTCTCAGGGCTCCCGGCATCCCCAGGGGTCCTTTC
GCAGGGGTCCCTATGCAGGAGGAGAACAGCCCAGAAAACAGGGAACTAGA
CCCTTGACAGGAAGTCCAAGGAGGGGTCCCTGGCTCACTGTGTGACCCTG
CTGGATCACTCGCCTCCGCTCTCGGGTCCCCTGAGCACTCCGTGCCTCCC
TTCCCTCCCCTAAAGTAAAAGCAGAAGTTAATCGCTTTCCCCTCCCCACG
CCCAACAAAGAGCAGGCCCTGTCCCCGGTGCTGAAGCGCCAGCCGCAGCG
CCTCCCCCACTCCCAAGGCATAAAACATGAGCCAAAACCAATAAAGAACC
AAATGTCACAGCTGTTGCAGGGCCCCCTAAGTCCCGGGGACCCCTTTTTC
TACCTGACATCCTAGTGGGGTGAGGGACTTTTGTACCTGGAAAGCATCCC
ATCACTTCCCTGGAAGCGAGAAGGGATGCCGACTCAGGCGCCTGCTTGTC
TGTTATGGGGGTAGGGGACCAGAGAACAAGTTGAGGCTGAGAAGATGGGG
AGGGGGAGGGAGAAAAGAGGACTTCATAGTGGCGAGAGAACGGCAAGATG
TGGGTTCCCCATCCCCAATTCAGCCAGAGACCCCTCAAAGTGGAACTTCC
TGGGGCAGTCGGGGGTCAGAAGTTGGAGCTTGTCTCTGGGGCAAGACCTC
TTCGTTGTACAGATGGAAAAACAAGGGTGGGAGAATACAGCTCGTCCAAG
GTCATTCGACTAGCAAACTGCTTAGCTGACCCTAGTGTGCAGAACCTGGC
TCGGGTGACACCCATCATTTCCCCCCACCCCACACAGGCGCCAGCTCTCT
CAATTTCATGCTCAAGCCCCGCTACGGTACCCCCACTGTGGGTTATCTGC
CCCTCAAACTCAGCCCAGCTTCCTCCTGCCTATTCGGGGAACCCTCTGCC
CGCTTCGCTGAGGGTCCGTCCCCTTTACTGGGGATGGCAGCAGGGTCTCC
TGTCTCCTCTCTCGGGGGGCCACTGCCGACTTTTCATAGAACGCTTTGCC
CCCTCCCAACCCCACCCATCCGGGGTTCCCTCTCTCCATCCTCTGCAGCG
TCTCCCATACCCCCATTGAGGGTAGTCTTGGTATTCTCCCCAACTCCAGG
TCCCCCTTCATCTATTCCAGGGCTGGCCGCGGAGTTTCCTGAGCGCTCTC
CAAGTGGGTCCTCTAGATGTTAGGAGAACACTGTACTTCCCCCCGTCAGG
GGTCTCCTGTCTCCGTTCTATGGAGCGTCCATGCTCCCATTCAGGACTGT
CTTGCTCCCTCCTCTATTCCGGGGCTGGCTGCACAGTCTCTGTACCCCCT
ATCCTGAGGGCCTCTCTTAACTATTTGGAAAGCCTCGTGTCCTCTCTCAT
ACGGGGATCCCTTCATCCTAATGACTGCAATCTTCCATTGCTCCATCCCT
AGGGCATCCTGCCCCTATTCCCATCAGGTTTCTCCTTGTCCTCTCCCTGT
TTCAAGTCCCCTTTCTTATTCCGAACACACTCTCAGGCTCTTCCGACGCA
TACCCGGGGGTCCTCACTGGCCCACTCCGGGAGTCCTCTGCCCGCTACCC
CGAACTCGGGGGTCTCCTCTGACGCAGCGTCGATTCCCCTTCCCTCCTCG
GTCCCCTACCCCGCCCCTCTCACTGCGGCTGAGCCGGTCAGCCGGGGGCC
GCAGGGGAGGAGGCGGAGAGGGCGGGGCCCTCCTCCCCACCCCCTCACTG
ACAAGGGGTTGGACCCGGCCGCGGCGGCTATAAAAGGGCCGGCGCCCTGG
TGCTGCCGCAGTGCCTCCAGCCCCGTCCCGGCCCCGCGCACCTGCTCCGG
C

As described above, in certain embodiments of the invention, the isolated nucleic acid molecule need not comprise a full length NEFH promoter sequence but may comprise, for example, only one, two, three, four or five of the Nefh promoter conserved regions A, D, F, B, C and E, or, for example only one, two, three or four of the NEFH promoter conserved regions A, D, F, D1, and K.

In another embodiment, the isolated nucleic acid molecule may comprise, for example, only two of the Nefh promoter conserved regions A, D, F, B, C and E, or, for example only two of the NEFH promoter conserved regions A, D, F, D1 and K separated by a spacer sequence such as a piece of lambda DNA such as that shown as SEQ ID NO:24.

SEQ ID NO: 24
SEQ ID NO: 24
AGGCATTTAT ACTCCGCTGG AAGCGCGTGT GTATTGCTCA CAATAATTGC
ATGAGTTGCC CATCGATATG GGCAACTCTA TCTGCACTGC TCATTAATAT
ACTTCTGGGT TCCTTCCAGT TGTTTTTGCA TAGTGATCAG CCTCTCTCTG
AGGGTGAAAT AATCCCGTTC AGCGGTGTCT GCCAGTCGGG GGGAGGCTGC
ATTATCCACG CCGGAGGCGG TGGTGGCTTC ACGCACTGAC TGACAGACTG
CTTTGATGTG CAACCGACGA CGACCAGCGG CAACATCATC ACGCAGAGCA
TCATTTTCAG CTTTAGCATC AGCTAACTCC TTCGTGTATT TTGCATCGAG
CGCAGCAACA TCACGCTGAC GCATCTGCAT GTCAGTAATT GCCGCGTTCG
CCAGCTTCAG TTCTCTGGCA TTTTTGTCGC GCTGGGCTTT GTAGGTAATG
GCGTTATCAC GGTAATGATT AACAGCCCAT GACAGGCAGA CGATGATGCA
GATAACCAGA GCGGAGATAA TCGCGGTGAC TCTGCTCATA CATCAATCTC
TCTGACCGTT CCGCCCGCTT CTTTGAATTT TGCAATCAGG CTGTCAGCCT
TATGCTCGAA CTGACCATAA CCAGCGCCCG GCAGTGAAGC CCAGATATTG
CTGCAACGGT CGATTGCCTG ACGGATATCA CCACGATCAA TCATAGGTAA
AGCGCCACGC TCCTTAATCT GCTGCAATGC CACAGCGTCC TGACTTTTCG
GAGAGAAGTC TTTCAGGCCA AGCTGCTTGC GGTAGGCATC CCACCAACGG
GAAAGAAGCT GGTAGCGTCC GGCGCCTGTT GATTTGAGTT TTGGGTTTAG
CGTGACAAGT TTGCGAGGGT GATCGGAGTA ATCAGTAAAT AGCTCTCCGC
CTACAATGAC GTCATAACCA TGATTTCTGG TTTTCTGACG TCCGTTATCA
GTTCCCTCCG ACCACGCCAG CATATCGAGG AACGCCTTAC GTTGATTATT
GATTTCTACC ATCTTCTACT CCGGCTTTTT TAGCAGCGAA GCGTTTGATA
AGCGAACCAA TCGAGTCAGT ACCGATGTAG CCGATAAACA CGCTCGTTAT
ATAAGCGAGA TTGCTACTTA GTCCGGCGAA GTCGAGAAGG TCACGAATGA
ACCAGGCGAT AATGGCGCAC ATCGTTGCGT CGATTACTGT TTTTGTAAAC
GCACCGCCAT TATATCTGCC GCGAAGGTAC GCCATTGCAA ACGCAAGGAT
TGCCCCGATG CCTTGTTCCT TTGCCGCGAG AATGGCGGCC AACAGGTCAT
GTTTTTCTGG CATCTTCATG TCTTACCCCC AATAAGGGGA TTTGCTCTAT
TTAATTAGGA ATAAGGTCGA TTACTGATAG AACAAATCCA GGCTACTGTG
TTTAGTAATC AGATTTGTTC GTGACCGATA TGCACGGGCA AAACGGCAGG
AGGTTGTTAG CGCGACCTCC TGCCACCCGC TTTCACGAAG GTCATGTGTA
AAAGGCCGCA GCGTAACTAT TACTAATGAA TTCAGGACAG ACAGTGGCTA
CGGCTCAGTT TGGGTTGTGC TGTTGCTGGG CGGCGATGAC GCCTGTACGC
ATTTGGTGAT CCGGTTCTGC TTCCGGTATT CGCTTAATTC AGCACAACGG
AAAGAGCACT GGCTAACCAG GCTCGCCGAC TCTTCACGAT TATCGACTCA
ATGCTCTTAC CTGTTGTGCA GATATAAAAA ATCCCGAAAC CGTTATGCAG
GCTCTAACTA TTACCTGCGA ACTGTTTCGG GATTGCATTT TGCAGACCTC
TCTGCCTGCG ATGGTTGGAG TTCCAGACGA TACGTCGAAG TGACCAACTA
GGCGGAATCG GTAGTA

### Vectors

As described above the promoter molecule and the expression cassette of the invention can be provided in a vector. In an embodiment, the promoter molecule and the expression cassette can be delivered to a cell using any suitable vector. For example, the vectors which may be used include viral and non-viral vectors, such as AAV serotypes, adenovirus, herpes virus, SV40, HIV, SIV and other lentiviral vectors, RSV and non-viral vectors including naked DNA, plasmid vectors, peptide-guided gene delivery, terplex gene delivery systems, calcium phosphate nanoparticles, magnetic nanoparticles, colloidal microgels and/or the integrase system from bacteriophage phiC31. Viral vectors useful in the invention include, but are not limited to, those listed in Table 1. Non-viral vectors useful in the invention include, but are not limited to, those listed in Table 2. Cationic lipid-based non-viral vectors can include glycerol-based (e.g. DOTMA, DOTAP, DMRIE, DOSPA), non-glycerol-based (e.g. DOGS, DOTIM) and/or cholesterol-based cationic lipids (e.g. BGTC, CTAP; Ju et al., 2015; Karmali and Chaudhuri, 2007; Lee et al., 2016). Viral and non-viral vector delivery may be accompanied by other molecules such as cationic lipids and/or polymers and/or detergents and/or agents to alter pH, such as, for example, polyethelene glycol (PEG), to enhance cellular uptake of vectors and/or to enhance expression from vectors and/or to evade the immune system. For example, polycationic molecules have been generated to facilitate gene delivery including but not exclusive to cationic lipids, poly-amino acids, cationic block co-polymers, cyclodextrins amongst others. Pegylation of vectors with polyethelene glycol (PEG) can shield vectors from, for example, the extracellular environment. Vectors may be used in conjunction with agents to avoid or minimise cellular immune responses such as PEG or as a Polyplex with Poly (L-Lysine) among others. Exosomes may be used to aid vector delivery and or evasion of the immune response (Wassmer et al., 2017). Vectors may be delivered in conjunction with immunomodulatory/immunosuppression regimes to aid transgene expression. Vector delivery may be undertaken using physical methodologies such as electroporation, nucleofection and/or ionotophoresis, either alone or in combination with molecules to enhance delivery. Vectors may be used in conjunction with agents to promote expression of transgenes incorporated into vectors, for example, using histone deacetylase inhibitors (HDAC) and/or DNA methyl transferase inhibitors and/or histone methyl transferase inhibitors to modulate chromatin structures thereby aiding expression. HDAC inhibitors include but are not exclusive to short chain fatty acids such as valproic acid and sodium butyrate, ketones, benzamides, cyclic and non-cyclic hydroxamates such as suberoyl anilide hydroxamic acids (SAHA), trichostatin A (TSA), cyclic peptides or tetrapeptides amongst others (Daly et al., 2016; Liu et al., 2006; Ververis et al., 2013). DNA methyl transfease inhibitors including, for example, 5-AC, decitabine and zebularine can be used to modulate chromatin structures. In addition, histone methyl transferase inhibitors can influence chromatin states, for example, BIX-01294 (diazepin-quinazolin-amine derivative). In addition, to the chemical entities referred to above, nucleic acids-based inhibitors can be used to suppress expression of proteins and/or non-coding RNAs involved in chromatin remodelling. In one embodiment of the invention vectors are optimized to specifically transduce target cell type(s) or target tissue type(s). Viral and/or non-viral vectors may be modified to target specific cell types and/or to prevent targeting of some cell types. For example, the inclusion of the capsid from AAV serotype 5 in an AAV2/5 hybrid virus facilitates transduction of photoreceptor cells or various serotypes including AAV2/2, AAV8BP2, 7m8 and others efficiently transduce RGCs, typically post intravitreal administration (Ramachandran et al., 2016). Similarly, for example, peptides may be included in viral vectors to facilitate targeting. Synthetic non-viral vectors can be modified to include ligands to facilitate targeting of vectors to specific cell and/or tissue types, for example, folate can be conjugated to liposomes to target tumour cells which over express the folate receptor (Hattori and Maitani, 2005; Lu and Low, 2012).

In another embodiment of the invention, vectors are designed to optimize the generation and/or production of vector, for example, to optimise viral titre and/or to optimize the number or type of nucleotides incorporated into vector(s). For example, vector genomes may be modified such that large transgenes may be incorporated into vectors, for example, ‘gutless’ adenovirus vectors have an increased capacity in terms of size than previous generations of adenovirus vectors. Components of vectors can be modified to optimize generation and production of vectors, for example, genes involved in replication of AAV can be modified to optimize replication and/or self complementary AAV vectors can be used to optimize rates of transgene expression. In an additional embodiment, vectors are designed to enable optimal expression of all components of a therapeutic. For example, where the vector is used to deliver two or more heterologous polynucleotide sequences, additional sequences can be included in the vector to optimize expression of each of the heterologous polynucleotide sequences. For example, vectors can include suppression and or replacement elements and or neurotrophic factor(s), among other nucleic acid components. For example where the vector is used to express two components, additional sequences can be included in the vector to optimize expression of both elements from a given vector. For example, inclusion of nucleotides to separate the ITRs of AAV and the nucleic acid component(s) can result in optimisation of expression of the components. Multiple nucleic acid components can be juxtaposed or separated from each other and/or can be in the same orientation or opposing orientations. Additional sequences, such as, for example, stuffer sequences can be included in vectors to optimize vector design. In addition, multiple nucleic acids components may be used in one vector. In addition vector design can include optimisation of codons to optimise levels of transgene expression, and/or achieve, modification of GC content, and/or removal of potential splice sites and/or other manipulations (Fischer et al., 2017).

TABLE 1
Exemplary Viral Vectors

| Delivery Method | Serotype | Reference |
|---|---|---|
| AAV | All serotypes, including | (Flannery et al., 1997; |
| | but not limited to 1, 2, | Lebkowski et al., 1988) |
| | 3, 4, 5, 6, 7, 8, 9, 10, | |
| | 11, 12, | |
| Lentivirus (for | VSV-G | (Balaggan et al., 2006; |
| example but not | Rabies-G | Pang et al., 2006; |
| exclusively | Further serotypes** | Takahashi, 2004) |
| Feline - FIV, | | |
| Equine - EIAV, | | |
| Bovine - BIV | | |
| and Simian - SIV). | | |
| Adenovirus | Various | (Bennett et al., 1996) |
| Simian papovirius | Various | (Kimchi-Sarfaty et al., |
| SV40 | | 2002) |
| Semliki Forest Virus | Various | (DiCiommo et al., 2004) |
| Sendai Virus | Various | (Ikeda et al., 2002) |

The list provided is not exhaustive; other viral vectors and derivatives, natural or synthesized could be used in the invention.

TABLE 2
Exemplary Non-Viral Vectors or Delivery Methods

| Delivery Method | Reference |
|---|---|
| Cationic liposomes | (Sakurai et al., 2001) |
| HVJ liposomes | (Hangai et al., 1998) |
| Polyethylenimine | (Liao and Yau, 2007) |
| DNA nanoparticles | (Farjo et al., 2006) |
| Dendrimers | (Marano et al., 2005) |
| Bacterial | (Brown and Giaccia, 1998) |
| Macrophages | (Griffiths et al., 2000) |
| Stem cells | (Hall et al., 2006) |
| Retinal transplant | (Ng et al., 2007) |
| Marrow/Mesenchymal | (Chng et al., 2007; |
| stromal cells | Kicic et al., 2003) |
| Implant (e.g., | (Montezuma et al., 2006) |
| Poly(imide)uncoated | |
| or coated) | |
| Electroporation | (Featherstone, 1993) |
| Targeting peptides | (Trompeter et al., 2003) |
| (for example but | |
| not exclusively Tat) | |
| Lipid mediated | (Zeng et al., 2007) |
| (e.g., DOPE, PEG) | |
| | (Amrite et al., 2006; |
| | Caplen et al., 1995) |
| | (Chalberg et al., 2005) |

The list provided is not exhaustive. Other non-viral vectors and derivatives, natural or synthesized and other delivery methods could be used with the invention.

In an embodiment, the heterologous polynucleotide encodes mammalian Myocilin 7, Opa1, Ndi1, rhodopsin, peripherin or others, such as those associated with diseases listed in Table 3. In another embodiment, the heterologous polynucleotide encodes neurotrophic factors, anti-apoptotic agents and/or antioxidants, such as those listed in Table 4.

TABLE 3
Diseases with known retinal ganglion cell/optic nerve involvement.

| Disease | Ocular symptoms/genes | References |
|---|---|---|
| Glaucoma | Optic nerve excavation, | (Weinreb et al., 2014) |
| | ganglion cell loss | |
| Multiple Sclerosis | Recurrent optic neuritis | (Chan, 2002) |
| Neuroretinitis | Transient inflammation of | (Purvin et al., 2011) |
| | the optic nerve head. | |
| Devic's disease | Inflammation and | (Weinshenker and |
| | demyelination of optic nerve | Wingerchuk, 2017) |
| | and spinal cord. | |
| Lupus | Optic nerve inflammation, | (Suri et al., 2016) |
| | treatable. | |
| Sarcoidosis | Optic neuritis, progressive | (Kidd et al., 2016) |
| | degeneration, optic nerve | |
| | involvement; can be bilateral. | |
| Wegener's | Optic nerve lesions. | (Purvin and Kawasaki, 2009; |
| Granulomatosis | | Takazawa et al., 2014) |
| Optic nerve tumours | Decreased visual function, | (Miller, 2004) |
| | proptosis, optic disc swelling | |
| | or pallor, and strabismus. | |
| | Central retinal vein occlusion | |
| | (CRVO), venous stasis | |
| | retinopathy, optociliary shunt | |
| | vessels, or rubeosis iridis with | |
| | neovascular glaucoma. | |
| Grave's disease | Autoimmune. Optic nerve | (So et al., 2000) |
| | compression by extraocular | |
| | muscles, progressive vision | |
| | loss, treatable with early | |
| | intervention. | |
| Anterior ischemic optic | Ischemia leading to axon | (Khalilpour et al., 2017) |
| neuropathy | degeneration and ganglion cell | |
| | loss. | |
| Toxic optic neuropathy | Optic nerve damage caused by | (Grzybowski et al., 2015) |
| | toxic compounds (ethylene | |
| | glycol, methanol, lead, tobacco | |
| | and alcohol (tobacco-alcohol | |
| | syndrome), insecticides, | |
| | chloramphenicol. | |
| Vitamin deficiency (B12, | Optic nerve pallor, scotoma, | (Chavala et al., 2005) |
| thiamine) | progressive, painless vision | |
| | loss. | |
| Ethambutol/Isoniazid | Transient optic neuritis, can | (Kass et al., 1957; |
| | resolve upon cessation of | Tsai and Lee, 1997) |
| | treatment | |
| Amiodarone/Digitalis | Progressive optic neuropathy | (Passman et al., 2012) |
| Leber's hereditary | Retinal ganglion cell | (Yu-Wai-Man et al., 2016) |
| optic neuropathy | degeneration; mitochondrial | |
| | inheritance (for example, | |
| | mutations in ND1, ND4, | |
| | ND4L, ND6). | |
| Dominant optic | Retinal ganglion cell | (Kerrison et al., 1999; |
| neuropathy | degeneration; autosomal | Reynier et al., 2004; |
| | dominant inheritance (for | Yu-Wai-Man et al., 2016) |
| | example, mutations in OPA1, | |
| | OPA3, OPA4, OPA5, OPA6, | |
| | OPA8) | |
| X-linked or recessive optic | Retinal ganglion cell | (Katz et al., 2006; |
| neuropathy | involvement (for example, | Lenaers et al., 2012) |
| | OPA2, OPA6 and OPA7 | |
| Friedreich's ataxia | Optic nerve pallor, retinal nerve | (Fortuna et al., 2009; |
| | fibre layer thinning. Mutations | Noval et al., 2012) |
| | in FXN. | |
| Mohr-Tranebjaerg | Deafness-dystonia-optic | (Jin et al., 1996) |
| syndrome | neuronopathy (DDON) | |
| | syndrome. Mutations in | |
| | DPP/TIMM8 | |
| Charcot-Marie-Tooth | Autosomal recessive disease, | (Bombelli et al., 2014) |
| disease | caused primarily by MFN2. | |
| | Optic atrophy seen as part of | |
| | disease progression in some | |
| | cases. | |
| Tay-Sachs | Sphingolipidosis. Progressive | (Chen et al., 2014) |
| | optic atrophy and retinal | |
| | degeneration; autosomal | |
| | recessive inheritance | |
| | (mutations in HEXA). | |
| Niemann-Pick | Sphingolipidosis. Progressive | (Chen et al., 2014) |
| | lipid deposits leading to retinal | |
| | ganglion cell degeneration; | |
| | autosomal recessive inheritance | |
| | (mutations in SMPD). | |
| Krabbe disease | Sphingoipidosis. Psychosine | (Chen et al., 2014) |
| | deposition leading to ganglion | |
| | cell layer cell loss and thinning; | |
| | autosomal recessive inheritance | |
| | (mutations in GALC). | |
| Leigh syndrome/NARP | Nerve fibre and ganglion cell | (Hayashi et al., 2000) |
| | layer thinning, optic nerve | |
| | atrophy; disease can be caused | |
| | by multiple genes, with strong | |
| | ocular involvement in MT- | |
| | ATP6 mutation (mitochondrial). | |
| Behr syndrome | Optic atrophy, spinocerebellar | (Bonneau et al., 2014) |
| | degeneration. Can present as | |
| | autosomal recessive or | |
| | dominant (for example, OPA1, | |
| | OPA3, C12ORF65). | |
| Wolfrum Syndrome | Autosomal recessive disorder | (Barrett et al., 1997; |
| | involving diabetes insipidus, | Hoekel et al., 2014) |
| | diabetes mellitus, optic atrophy, | |
| | and deafness (DIDMOAD) (for | |
| | example, WFS1). Wolfrum | |
| | Syndrome 2 involving optic | |
| | atrophy, diabetes mellitus, | |
| | deafness and decreased lifespan | |
| | can be caused by mutations in | |
| | the CISD2 gene. | |

The list provided is not exhaustive.

TABLE 4
Exemplary neurotrophic factors, anti-apoptotic agents and antioxidants,
which may be used in conjunction with the promoter described
herein. These genes may be delivered at the same time as a
therapeutic gene listed in Table 3 or at a different time,
using the same vector or a different vector.

| | Reference |
|---|---|
| Neurotrophic factor | |
| NGF | (Carmignoto et al., 1989) |
| b-NGF | (Lipps, 2002) |
| NT-3 | (Lu et al., 2011) |
| NT4 | (Bikbova et al., 2013) |
| BDNF | (Carmignoto et al., 1989; |
| | Di Polo et al., 1998) |
| GDNF | (Frasson et al., 1999; |
| | Gregory-Evans et al., 2009; |
| | Wu et al., 2004) |
| NTN (Neurturin) | (Koeberle and Ball, 2002) |
| aFGF and bFGF | (Akimoto et al., 1999; |
| | Faktorovich et al., 1990; |
| | Lau et al., 2000; |
| | McLaren and Inana, 1997; |
| | Uteza et al., 1999) |
| LIF | (Joly et al., 2008; |
| | Rhee and Yang, 2010) |
| CNTF | (Li et al., 2011; |
| | Sieving et al., 2006) |
| Hepatocyte growth factor | (Tönges et al., 2011) |
| PDGF | (Akiyama et al., 2006) |
| VEGF | (Trujillo et al., 2007) |
| PEDF | (Cayouette et al., 1999) |
| RdCVF | (Léveillard et al., 2004) |
| Chondroitinase ABC | (Liu et al., 2012) |
| Erythropoietin | (Rex et al., 2009; |
| | Rong et al., 2011; |
| | Sullivan [symbol] al., 2011) |
| Suberythropoietc Epo | (Wang et al., 2011) |
| Anti-apoptotic agents | |
| Calpain inhibitor I | (McKernan et al., 2007) |
| Calpain inhibitor II | (McKernan et al., 2007) |
| Calpeptin | (McKernan et al., 2007) |
| PARP | |
| Norgestrel | (Doonan et al., 2011) |
| Antioxidant | |
| Vitamin C | www.nei.nih.gov/amd |
| Vitamin E | www.nei.nih.gov/amd |
| Beta-carotene | www.nei.nih.gov/amd |
| SOD2 +/− catalase | (Doonan et al., 2009; |
| | Usui et al., 2009) |
| Rosiglitazone | (Doonan et al., 2009) |
| Sestrin-1 | (Budanov et al., 2004) |
| PPAR | (Aoun et al., 2003; |
| | Fan et al., 2008; |
| | Tomit [symbol] et al., 2005; |
| | Zhao et al., 2006) |
| Lutein | (Li and Lo, 2010) |

[symbol]

indicates data missing or illegible when filed

The list provided is not exhaustive.

TABLE 5
Exemplary enhancer elements and epigenetic elements

| | Reference |
|---|---|
| Enhancer Element | |
| Chicken ovalbumin upstream promoter | (Eguchi et al., 2007) |
| transcription factor II | |
| Mouse dystrophin muscle promoter/enhancer | (Anderson et al., 2006) |
| Tobacco eIF4A-10 promoter elements | (Tian et al., 2005) |
| Immunoglobulin (Ig) enhancer element | (Frezza et al., 2007) |
| HS1, 2A | |
| Col9al enhancer element | (Genzer and Bridgewater, |
| | 2007) |
| Gata2 intronic enhancer | (Khandekar et al., 2007) |
| TH promoter enhancer | (Gao et al., 2007) |
| CMV enhancer | InvivoGen cat# pdrive-cag |
| | 05A13-SV |
| Woodchuck hepatitis virus | (Donello et al., 1998; |
| posttranscriptional regulatory element | Schambach et al., 2006) |
| IRBP | (Ying et al., 1998) |
| CMV enhancer and chicken β-actin | InvivoGen cat# pdrive-cag |
| promoter | 05A13-SV |
| CMV enhancer and chicken β-actin | InvivoGen cat# pdrive-cag |
| promoter and 5′UTR | 05A13-SV |
| CpG-island | (Antoniou et al., 2003) |
| Epigenetic elements | |
| Mcp Insulators | (Kyrchanova et al., 2007) |
| CpG-island region of the HNRPA2B1 | (Williams et al., 2005) |
| locus | |
| Chicken b-globin 5′hypersensitive site 4 | (Kwaks and Otte, 2006) |
| (cHS4) | |
| Ubiquitous chromatin opening elements | (Kwaks and Otte, 2006) |
| (UCOEs) | |
| Matrix associated regions (MARs) | (Kwaks and Otte, 2006) |
| Stabilising and antirepressor elements | (Kwaks and Otte, 2006) |
| (STAR) | |
| Human growth hormone gene silencer | (Trujillo et al., 2006) |

This list is not exhaustive

In an embodiment of the invention, the invention may be used to direct expression of heterologous polynucleotides to RGCs to provide transgene expression in these cells and/or to alleviate disease pathology. In another embodiment, the invention may be used to drive expression in RGCs, for example, to express a marker gene. The nucleic acid molecules, expression cassettes and vectors of the invention may thus be used in methods to identify a RGC. Kits comprising the isolated nucleic acid molecule according to the first aspect of the invention, the expression cassette according to the second aspect of the invention, or the vector according to the third aspect of the invention. This would enable, by inclusion of a marker gene in the kit, the identification of RGC cells for subsequent sorting or staining or isolation.

### Cells

In another aspect, the invention provides cells comprising a promoter sequence of the first aspect of the invention, an expression cassette of the second aspect, or a vector of the third aspect for experimental or therapeutic use. In an embodiment, the cells express a suppressor such as antisense, and or RNAi that can target a gene expressed in RGCs. In another embodiment, the cells express a replacement nucleic acid. In another embodiment, the cells express a nucleic acid to augment expression of an endogenous gene and or to provide expression of a nucleic acid not normally expressed in that cell type. In another embodiment, the cells express a replacement nucleic acid that is not targeted by the suppressor. In an embodiment, the cells express a gene editing component such as CRISPR/Cas that can target a gene expressed in RGCs. In another embodiment, the cells comprise a vector encoding at least one or more suppression and or gene editing component(s). In another embodiment, the cells comprise a vector encoding one or more nucleic acids. In an additional embodiment, the cells comprise one or more vectors encoding suppression and or gene editing component(s) and or replacement nucleic acid(s).

In another aspect, the invention provides a transgenic animal comprising the isolated nucleic acid molecule according to the first aspect of the invention, the expression cassette according to the second aspect of the invention, the vector according to the third aspect of the invention, or the cell according the fourth aspect of the invention and its experimental or therapeutic use. In an embodiment, the transgenic animal is a model for Leber Hereditary Optic Neuropathy (LHON). In another embodiment, the transgenic animal is a model for dominant optic atrophy (DOA). In another embodiment, the transgenic animal is a model for glaucoma.

The isolated nucleic acid molecule according to the first aspect of the invention, the expression cassette according to the second aspect, or the vector of the third aspect of the invention can be administered to cells, tissues, and/or animals. Administration of the isolated nucleic acid molecule, the expression cassette, or the vector may be systemic or local. Administration of the isolated nucleic acid molecule, the expression cassette, or the vector may be used in conjunction with chemical and/or physical agents to aid administration. In a particular embodiment, the isolated nucleic acid molecule, the expression cassette, or the vector is for administration by intraocular (e.g., subretinal and/or intravitreal) injection. In the case of the retina, intravitreal injection can be used to administer a polynucleotide according to the following procedure. For example, mice can be anaesthetised by intraperitoneal injection of Domitor and Ketalar (10 and 50 μg/g of body weight respectively). The pupils can be dilated with phenylephrine and under local analgesia (amethocaine) a small puncture is made in the sclera. A micro-needle attached to a 10 μl syringe (Hamilton Company Europe) is inserted through the puncture to the vitreous and, for example, 1-3 μl of vector can be administered into the vitreous. For example, in the case of AAV 1-3 μl of a 10⁹⁻¹⁴ vp/ml AAV vector preparation in PBS is administered. A reverse anaesthetic (antisedan, 50 μg/g of body weight) can be applied by intraperitoneal injection post-delivery. Body temperature during the procedure can be sustained using a homeothermic heating device. In addition newborn mice can be prepared for intravitreal injection according to Matsuda and Cepko or injected in utero according to Dejnenka et al. and Garcia-Frigola et al. (Dejneka et al., 2004; Garcia-Frigola et al., 2007; Matsuda and Cepko, 2004; Patrício et al., 2017).

In one embodiment of the invention administration of the isolated nucleic acid molecule, the expression cassette, and or the vector in combination with one or more factors to facilitate cell survival, cell viability and/or cell functioning is contemplated. In relation to neurons, a range of neurotrophic and/or neuroprotective factors may be used, including brain derived neurotrophic factor (BDNF), glial derived neurotrophic factor (GDNF), neurturin, ciliary derived neurotrophic factor (CNTF), nerve growth factor (NGF), fibroblast growth factors (FGF), insulin-like growth factors (IGF), pigment epithelium-derived factor (PEDG), hepatocyte growth factor (HGF), thyrotrophin releasing hormone (TRH) and rod derived cone viability factor (RDCVF) amongst others (Feng et al., 2017; Igarashi et al., 2016; Kimura et al., 2016; Koeberle and Ball, 2002; Ortín-Martínez et al., 2014; Rathnasamy et al., 2017). There is substantial evidence in the literature that such factors may increase cell viability and/or cell survival for a range of cell types. For example, these factors have been shown to provide beneficial effects to a wide range of neuronal cell types including, for example, RGCs and or photoreceptors, when delivered either in protein or DNA forms (Buch et al., 2006; Cen et al., 2017; Feng et al., 2017). The use of GDNF to augment gene-based therapies for recessive disease has been demonstrated in mice (Buch et al., 2006; Feng et al., 2017). Genes encoding neurotrophic/neuroprotective factors may be expressed from general promoters such as the CBA promoter (Buch et al., 2006) or from tissue specific promoters such as the promoter sequence of the invention or promoter elements from genes detailed in Table 7 (and/or Table 3). Sequences to optimise expression of neurotrophic/neuroprotective factors such as those sequences identified in Table 4, may be included in constructs.

In one embodiment of the the isolated nucleic acid molecule, the expression cassette, and or the vector may be administered in combination with one or more factors to facilitate mitochondrial function including but not limited to Ndi1, Opa1, ND1, ND4, ND6, NDUAF6, and or AOX. In another embodiment administration of the isolated nucleic acid molecule, the expression cassette, and or the vector in combination with a corrected or optimised version of one or more of the genes causative of the disorders including but not limited to those listed in Table 3.

### Evaluation of Expression of Heterologous or Endogenous Genes Using RNA Assays

Expression of heterologous polypeptides (genes of interest) and/or endogenous genes can be evaluated in cells, tissues and/or animals using RNA assays including real time RT-PCR, northern blotting, RNA in situ hybridisation and or RNAse protection assays. RNA expression levels of heterologous and/or endogenous nucleic acids can be assessed by real time RT-PCR using, for example, a Step-One Real Time PCR System (Applied Biosystems, Foster City, CA, USA) and using, for example, a QuantiTect SYBR Green RT-PCR kit (Qiagen Ltd). RT-PCR assays are undertaken using levels of expression of housekeeping controls such as B-actin or GAPDH, for example, for comparative purposes. Levels of RNA expression can be evaluated using sets of primers targeting the nucleic acids of interest. For example, the following primers can be used for the evaluation of levels of expression of Thy1, gamma-synuclein, Ndi1, GDNF, Brn3a, Nefh, rhodopsin, channelopsins, EGFP, β-actin, GAPDH, melanopsin, among others.

TABLE 6
Examples of PCR Primers for measuring
rhodopsin, β-actin, GAPDH, Nefh (two primer pairs given),
gamma-synuclein, Brn3a, Thy1 and Melanopsin

| Primer | Sequence | SEQ ID NO |
|---|---|---|
| RHO forward primer | 5′ CTTTCCTGATCTGCTGGGTG 3′ | SEQ ID NO: 25 |
| RHO reverse primer | 5′ GGCAAAGAACGCTGGGATG 3′ | SEQ ID NO: 26 |
| EGFP forward | 5′ TTCAAGAGGACGGCAACATCC 3′ | SEQ ID NO: 27 |
| primer | | |
| EGFP reverse primer | 5′ CACCTTGATGCCGTTCTTTCGC 3′ | SEQ ID NO: 28 |
| β-actin forward | 5′ TCACCCACACTGTGCCCATCTACGA 3′ | SEQ ID NO: 29 |
| primer | | |
| β-actin reverse | 5′ CAGCGGAACCGCTCATTGCCAATGG 3′ | SEQ ID NO: 30 |
| primer | | |
| GAPDH forward | 5′-CAGCCTCAAGATCATCAGCA-3′ | SEQ ID NO: 31 |
| primer: | | |
| GAPDH reverse | 5′-CATGAGTCCTTCCACGATAC-3′ | SEQ ID NO: 32 |
| primer: | | |
| Nefh forward primer | 5′-TGGCCCTGGACATTGAGATT-3′ | SEQ ID NO: 33 |
| 1: | | |
| Nefh reverse primer | 5′-TGCGTGGATATGGAGGGAAT-3′ | SEQ ID NO: 34 |
| 1: | | |
| Nefh forward primer | 5′-ACCGTCATCAGGCAGACATT-3′ | SEQ ID NO: 35 |
| 2: | | |
| Nefh reverse primer | 5′-AATGTCCAGGGCCATCTTGA-3′ | SEQ ID NO: 25 |
| 2: | | |
| Gamma synuclein | 5′-TCTCCATTGCCAAGGAAGGT-3′ | SEQ ID NO: 26 |
| forward primer: | | |
| Gamma synuclein | 5′- CTTGTTGGCCACTGTGTTGA-3′ | SEQ ID NO: 27 |
| reverse primer: | | |
| Brn3a forward | 5′-CGCAGCGTGAGAAAATGAAC-3′ | SEQ ID NO: 28 |
| primer: | | |
| Brn3a reverse | 5′-TGGCAGAGAATTTCATCCGC-3′ | SEQ ID NO: 29 |
| primer: | | |
| Thy1 forward | 5′-TGAACCAAAACCTTCGCCTG-3′ | SEQ ID NO: 30 |
| primer: | | |
| Thy1 reverse primer: | 5′-AGCTCACAAAAGTAGTCGCC-3′ | SEQ ID NO: 31 |
| Melanopsin forward | 5′-GGGTTCTGAGAGTGAAGTGG-3′ | SEQ ID NO: 32 |
| primer: | | |
| Melanopsin reverse | 5′-AAGAGGCCTTGAGTTCTCC-3′ | SEQ ID NO: 33 |
| primer: | | |

Expression of heterologous or endogenous genes may be confirmed, for example, by Northern blotting or real time RT qPCR. Real time RT PCR may be performed using standard methodologies such as those described in O'Reilly et al., 2007 and using primers such as amongst others those listed in Table 6.

RNA may also be detected by in situ hybridisations using single stranded RNA probes that have been labelled with, for example, DIG. To evaluate levels of expression of heterologous genes or endogenous genes, RNase protections assays can be performed using art known methods, such as that described in the Ambion mirVana™ Probe and Marker kit manual and the Ambion RPAIII™ Ribonuclease protection assay kit manual, as described (Chadderton et al., 2009; O'Reilly et al., 2007). For example, RNA probes approximately 15-25 nucleotides in length specific for transcripts from, for example, a heterologous gene can be synthesized.

Expression of heterologous genes and/or endogenous genes can be undertaken and determined in cells, in tissues and or in animals using, for example, the assays and associated methodologies provided above.

### Evaluation of Expression of Heterologous and Endogenous Genes Using Protein Assays

Expression of heterologous genes and/or endogenous genes can be evaluated in cells, tissues and/or animals using protein assays including ELISA, western blotting and immunocytochemistry assays. ELISAs can be undertaken to evaluate levels of expression of a target endogenous gene-such proteins assays are well know in the art and methods are provided in, for example Palfi et al. (2006). For example, in the case of retinal genes such as the rhodopsin gene, ELISA is undertaken using a rhodopsin primary antibody which is typically used in a diluted form, for example, using a 1/10-1/10000 dilution (but possibly outside of this range) of an antibody for the target protein. Antibodies including Thy1, Ndi1, including others, can be used to evaluate levels of endogenous and/or heterologous genes expressed in RGCs. In addition, Western Blotting may be undertaken to determine relative quantities of a specific protein, for example GDNF, Ndi1, Thy1 and others. Briefly, protein samples are separated using SDS-PAGE and transferred to a membrane. The membrane is incubated with generic protein (for example milk proteins) to bind to “sticky” places on the membrane. A primary antibody is added to a solution which is able to bind to its specific protein and a secondary antibody-enzyme conjugate, which recognises the primary antibody is added to find locations where the primary antibody bound.

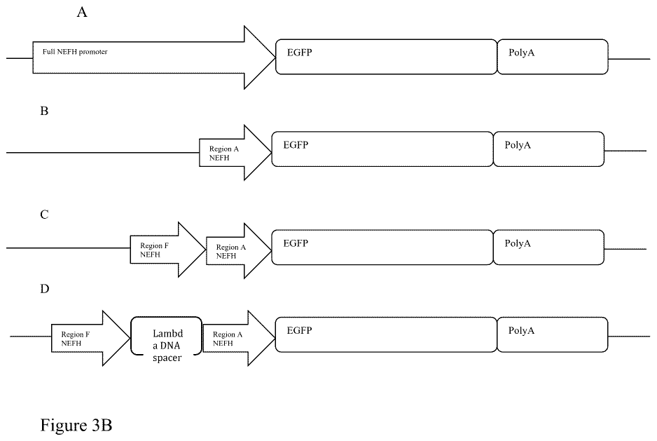

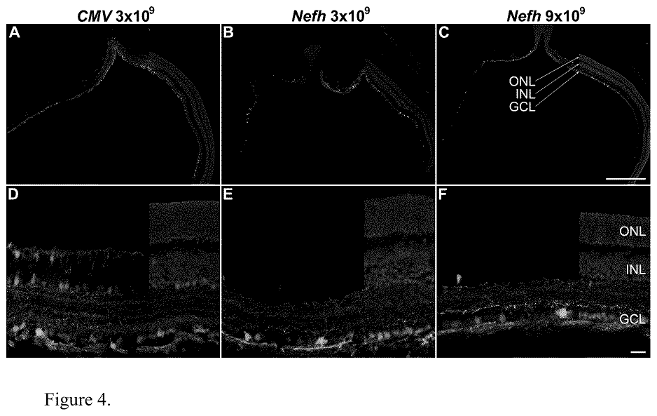

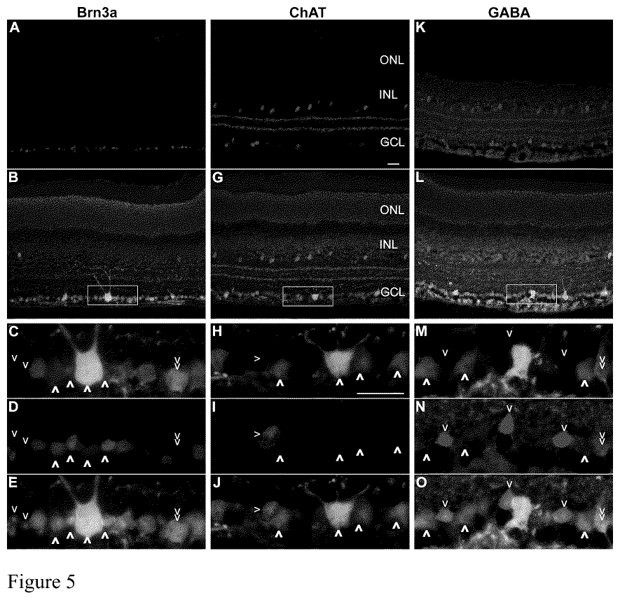

In addition to the protein assays referred to above, assays using antibodies in conjunction with microscopy can be used to evaluate protein levels. For example, in the case of Brn3a, GABA, EGFP, or rhodopsin immunocytochemistry (for example, using a 1/10-1:1000 dilution of a primary antibody) and fluorescent microscopy can be carried out as has been documented, and in FIGS. 4A-4F and 5A-5O below (Chadderton et al., 2012; Kiang et al., 2005). Immunocytochemistry can be undertaken on cells and/or tissues. In the case of the retina, various modes of sectioning can be implemented to evaluate retinal sections. For example, frozen sections, agar embedded sections and/or resin embedded sections can be used. To obtain thin sections, for example of the retina, epon embedding and semi-thin sectioning can be performed using art known methods such as those provided in Chadderton et al. (2012); McNally et al. (2002). Histological analyses can be used to evaluate the histological effect(s) associated with the administration of the nucleic acid components of the invention. In wild type or animal models with a retinal degeneration histological analyses can be used to evaluate administration of heterologous or augmentation of endogenous genes of interest.

### Delivery of Heterologous or Endogenous Polynucleotides

Both non-viral and/or viral vectors can be used in the invention to deliver the heterologous and/or endogenous polynucleotides of interest in expression cassettes of the second aspect of the invention. For example, in the case of retina, recombinant adenoassociated virus (AAV) and more specifically AAV2/2 may be used to elicit efficient preferential transduction of RGCs. Other AAV serotypes may also be used to deliver to retina, for example, AAV2/2 elicits efficient delivery to the retinal pigment epithelium (RPE), as does AAV4. AAV vectors can be generated using protocols with and without helper virus. For example, a helper virus free protocol using a triple transfection approach is well documented (Xiao et al., 1998). Expression cassettes can be cloned into plasmids such as pAAV-MCS provided by Stratagene Inc. Transgenes can be cloned between the inverted terminal repeats of AAV2 and transfected into 293 cells (Agilent; ATCC cat no CRL-1573) with two other plasmids, hence the term triple transfection. For example, the pRep2/Cap2 plasmid (Agilent) together with the pHelper plasmid (Agilent), at, for example, a ratio of 1:1:2, can be used to generate AAV2/2 vectors. Virus can be generated using a variety of art known procedures including the method outlined below. For example, to generate virus fifty 150 mm plates of confluent HEK293 cells were transfected (50 ug DNA/plate) with polyethyleminine (Reed et al., 2006). 48 hrs post-transfection crude viral lysates were cleared (Auricchio et al., 2001) and purified by CsCl2 gradient centrifugation (Zolotukhin et al., 1999). The AAV containing fraction was dialysed against PBS. Genomic titres, viral particles (vp/ml), were determined by quantitative real-time PCR using art known methods (Rohr et al., 2002).

### Assay for Function

To evaluate if administration of a heterologous and/or augmentation of an endogenous gene using an expression cassette or a vector of the invention modulates the function of a target tissue and/or cell type, one or more assays may be employed that are well described in the prior art. In the case of the retina, functional assays include but are not limited to electrophysiological assays including the full-field electroretinogram (ERG) and the pattern electroretinogram (PERG) and psychophysical assays such as visual field assessment, both kinetic and static, colour vision testing, and pupillometry. Protocols for ERG and PERG recording in humans have been established by the International Society for Clinical Electrophysiology of Vision (ISCEV) and may be adapted for similar recording in animals. The full-field ERG can be performed using, for example, the following procedure or an adapted procedure. Animals are dark-adapted overnight and prepared for ERG under dim red light. Pupils are dilated with 1% cyclopentalate and 2.5% phenylephrine. Animals are anesthetized with ketamine and xylazine (16 and 1.6 μg/10 g body weight respectively) injected intraperitoneally. Standardized flashes of light are presented to the animal, for example a mouse, in a Ganzfeld bowl. ERG responses are recorded simultaneously from both eyes by means of contact lens, gold wire or saline impregnated cotton thread electrodes, amongst others, using 1% amethocaine as topical anaesthesia. Reference and ground electrodes are positioned subcutaneously, approximately one mm from the temporal canthus and anterior to the tail respectively. Responses are analysed using appropriate recording equipment. Rod-isolated responses are recorded using a dim white flash (−25 dB maximal intensity where maximal flash intensity was 3 candelas/m²/s) presented in the dark-adapted state. Maximal combined rod-cone responses to the maximal intensity flash are then recorded. Following a 10 minute light adaptation to a background illumination of 30 candelas/m², cone-isolated responses are recorded to the maximal intensity flash presented initially as a single flash and subsequently as 30 Hz flickers in humans or 10 Hz in mice. A-waves are measured from the baseline to the trough and b-waves from the baseline (in the case of rod-isolated responses) or from the a-wave to the trough. The amplitude as well as the timing of the waveforms can provide valuable on both rod and cone photoreceptor function. The photopic electroretinogram negative response (PhNR), a component that follows the b-wave peak of the photopic full-field ERG, is thought to be correlated with inner retinal activity, particularly RGC activity, and is selectively reduced in optic neuropathies. The Visual Evoked Potential (VEP) assesses the transmission of electrical signals, predominantly generated by the macula, to the visual cortex. This response is, in fact, measured by electrodes placed over the occipital visual cortex, the exciting stimulus being either checkerboard pattern stimuli or flash stimuli. The amplitude of the signal correlates with the number of healthy retinal cells contributing to the signal of the signal while the efficiency of transmission along the optic nerve pathway may assays by determination of the latency of the signal, delay indicating pathological disturbance of transmission.

### Optokinetics

OKR spatial frequency thresholds are typically measured blind by two independent researchers using a virtual optokinetic system (VOS, OptoMotry, Cerebral Mechanics, Lethbridge, Alberta, Canada) as described (Prusky et al., 2004). OptoMotry measures the threshold of the mouse's optokinetic tracking response to moving gratings. Briefly, a virtual-reality chamber is created with four 17-inch computer monitors facing into a square and the unrestrained mouse placed on a platform in the centre. A video camera, situated above the animal, provides real-time video feedback. The experimenter centres the virtual drum on the mouse's head and judges whether the mouse makes slow tracking movements with its head and neck. The spatial frequency threshold, the point at which the mouse no longer tracks, is obtained by incrementally increasing the spatial frequency of the grating at 100% contrast. A staircase procedure is used in which the step size is halved after each reversal, and terminated when the step size becomes smaller than the hardware resolution (~0.003c/d, 0.2% contrast). One staircase is presented for each direction of rotation to measure each eye separately, with the two staircases being interspersed.

### Magnetic Resonance Imaging

Optic nerve integrity in experimental and control mice can be assessed by MEMRI (Bearer et al., 2007; Lin and Koretsky, 1997; Lindsey et al., 2007; Watanabe et al., 2001) using a 7-T Bruker Biospec 70/30 magnet (Bruker Biospin, Etlingen, Germany). MEMRI demarcates active regions of the brain due to the ability of Mn²⁺ ions to enter excitable cells through voltage-gated calcium channels. Thus analysis of Mn²⁺ transport through the optic nerve provides a good measure of its integrity. Two hours before scanning, mice are anaesthetised and intravitreally injected, as described (Chadderton et al., 2012), with 2 ml of 20 mg/ml manganese chloride (MnCl₂) in phosphate buffered saline (PBS). Log signal intensities from MRI scans corresponding to the region immediately superior to the optic chiasm can be quantified using the Image J software (Abramoff et al., 2004) (imagej.nih.gov/ij/). Assays that may be used to assess transgene expression and functional effects are not limited to the assays detailed above.

The agents of the invention (e.g. isolated nucleic acids, expression cassettes and/or vectors) may be administered in effective amounts. An effective amount is a dosage of the agent sufficient to provide expression of the transgene and or a medically desirable result. An effective amount means that amount necessary to delay the onset of, inhibit the progression of or halt altogether the onset or progression of the particular condition or disease being treated and/or provide expression of a marker or molecular tool. An effective amount may be an amount that reduces one or more signs or symptoms of the disease. When administered to a subject, effective amounts will depend of course on the particular condition being treated; the severity of the condition; individual patient parameters including age, physical condition, size and weight, concurrent treatment, frequency of treatment, and the mode of administration. These factors are well known to those of ordinary skill in the art and can be addressed with no more than routine experimentation.

Actual dosage levels of active ingredients in the compositions of the invention can be varied to obtain an amount of the agent(s) that is effective to achieve the desired therapeutic response for a particular patient, composition, and mode of administration. The selected dosage level depends upon the activity of the particular agent, the route of administration, the severity of the condition being treated, the condition, and prior medical history of the patient being treated. However, it is within the skill of the art to start doses of the agent(s) at levels lower than required to achieve the desired therapeutic effort and to gradually increase the dosage until the desired effect is achieved.

Practice of the invention will be still more fully understood from the following examples, which are presented herein for illustration only and should not be construed as limiting the invention in any way.

### EXEMPLIFICATION

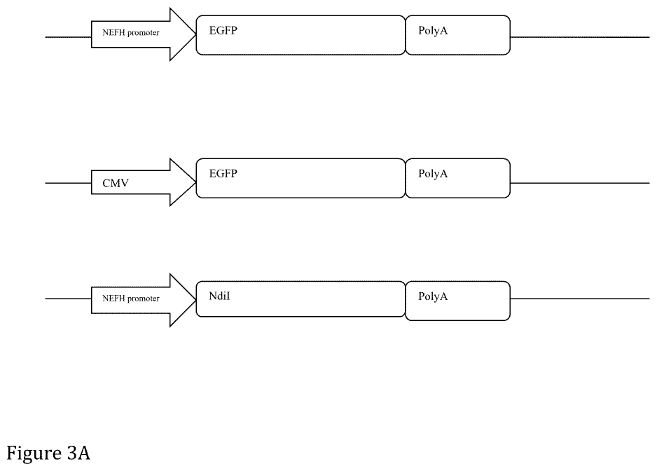

To help illustrate the current invention, five constructs were generated using different conserved regions of murine Nefh or human NEFH upstream sequence to drive EGFP marker gene expression (FIGS. 3B and 3C). From these constructs AAV 2/2 vectors were generated, though other vectors can also be utilised, to test for expression in murine retinas following intravitreal injection. These constructs were termed AAV.NEFH-EGFP (SEQ ID NO: 128), AAV.Nefh-EGFP (SEQ ID NO: 129), AAV.A-EGFP (SEQ ID NO: 124), AAV.A+F-EGFP (SEQ ID NO: 126), and AAV.A-spacer-F-EGFP (SEQ ID NO: 125).^{~}

### Example 1

### In Silico RGC Promoter Analyses

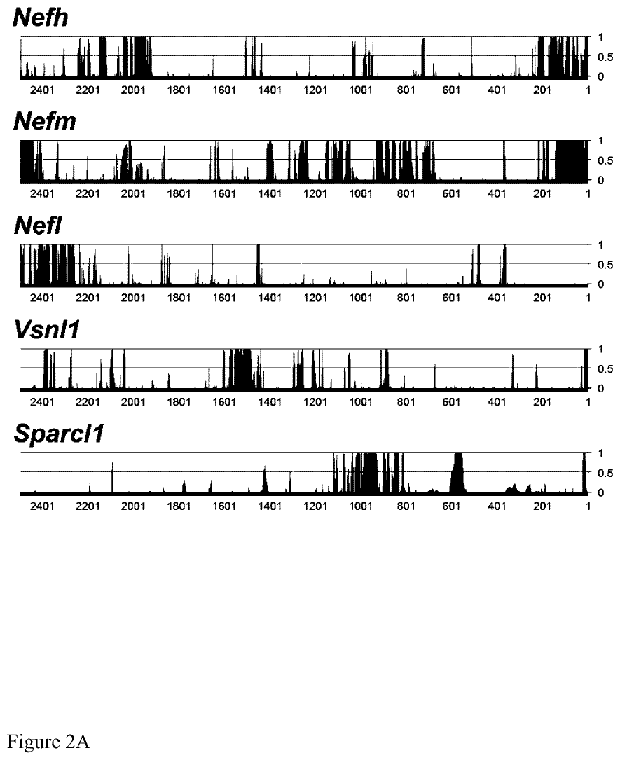

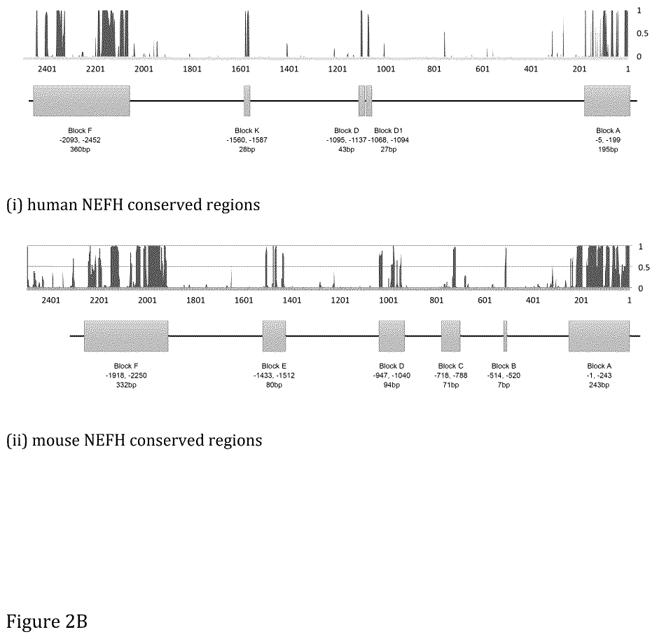

Human genes whose relative expression was enriched in the RGC layer by over 10-fold compared to relative expression in OR were selected (Kim et al., 2006). Genes were assessed based on GCL expression level (EL_{GCL}) compared to the OR expression (EL_{OR}), termed the enrichment factor (EF=EL_{GCL}/EL_{OR}; Kim et al., 2006) and the 15 genes with the highest ELs were selected for further investigation. A gene score (GS=EL_{GCL}×EF) was used to rank genes for suitability as potential promoters. Further analysis was performed on mouse genomic data, as a mouse promoter was the desired output. Data from the UCSC genome browser (mm10 mouse mammalian conservation track; UCSC; Kent et al., 2002) were used to establish conservation upstream of the transcriptional start site of candidate genes; results from analysis of 2.5 kb upstream of the start site are presented (FIG. 2A and FIG. 2B). An in silico pipeline (Python) was developed to isolate basewise conservation data from UCSC (conservation data ranged from 0 to 1 for a given base, where 0 represents no significant conservation between mammals and 1 indicates complete conservation). The forty mammalian species and their sequence assembly names that make up this conservation data are listed in Table 8. This was plotted in a graph in order to visualise conserved regions. NEFH was chosen as having the highest GS of the genes analysed. Using the parameters defined above, the mouse Nefh upstream region was selected for evaluation in vivo, given the expression profile of the gene and conservation of its 5′ upstream sequence.

### Cloning and AAV Production

pAAV.CMV-EGFP was cloned as described (Palfi et al., 2010). To generate pAAV.Nefh-EGFP, a 2251 bp fragment of mouse Nefh upstream sequence (NM_010904.3) was amplified from genomic DNA and substituted for the CMV promoter in pAAV.CMV-EGFP. To create pAAV.minNefh-EGFP a 838 bp fragment encompassing the six highly conserved regions of Nefh was synthesized by Integrated DNA Technologies (IDT) and substituted for the CMV promoter in pAAV.CMV-EGFP. pAAV.NEFH-EGFP was generated by amplifying ~1.9 kb fragment of human genomic DNA (NM_021076.3) using the following primers: Forward primer: 5′ AGATCATCTTAAGACGCGTTGCTGTCAGCTGCTTGTGA 3′ (SEQ ID NO: 45) and Reverse primer: 5′GAGGTACAGTGTTCTCCTAAC 3′ (SEQ ID NO: 46). The purified PCR product was cloned into pcDNA3.1+ (Invitrogen) along with a fragment of custom synthesized DNA obtained from GeneWiz in their standard vector (pUC57-Amp; see below). The full length NEFH, 2501 bp, was excised and cloned in place of the CMV promoter in pAAV-CMV-EGFP to create pAAV-NEFH-EGFP (SEQ ID NO: 47).

AGTTTCCTGAGCGCTCTCCAAGTGGGTCCTCTAGATGTTAGGAGAAC
ACTGTACCTCCCCCGGTCAGGGGTCTCCTGTCTCCGTTCTATGGAGC
GTCCATGCTCCCATTCAGGACTGCCTTGCTCCCTCCTCTGTTCCGGG
GCTGGCTGCACAGTCTCTGCACCCCCTATCCTGAAAGCCTCTCTTAA
CTATTTGGAAAGCCTCGTGTCCTGTCTCATACAGGGATCCCCTCATC
CTAATGACTGCAATCTTCCATTGCTCCATCCCGAGGGCATCCTGCCC
CTATTCCCATCAGGTTTCTCCTTGTCCTCTCCCTGTTTCAAGTCCCC
TTTCTTATTCCGAACACACTCGCAGGCTCTTCCGACGCGCACCCGGG
GGTCCTCACTGGCCCACTCCGGGAGTCCTCTGCCCGCTTCCCCGACC
TCGAGGGTCTCCTCTGACGCAGCGTCGATTCCCCTTCCCTCCTCGGT
CCCCTGCCCCGCCCCTCTCACTGCGGCGGAGCCGGTCGGCCGGGGGG
CCGCAGGGGAGGAGGCGGAGAGGGCGGGGCCCTCCTCCCCACCCTCT
CACTGCCAAGGGGTTGGACCCGGCCGCGGCGGCTATAAAAGGGCCGG
CGCCCTGGTGCTGCCGCAGTGCCTCCCGCCCCGTCCCGGCCTCGCGC
ACCTGCTCAGCGATATCCTAGGAATTCAGCTTCTAGA

To create pAAV.A-EGFP, conserved region A was amplified from human genomic DNA using the following primers: Forward: 5′-ATCGATGACGCGTCTCTGACGCAGCGTCGATT-3′ (SEQ ID NO: 48); and Reverse: 5′-AGATCATGATATCGGCCTGAGCAGGTGCGCGA-3′ (SEQ ID NO: 49) and cloned upstream of EGFP in pAAV-MCS-EGFP (Agilent Technologies) was digested with MluI and EcoRV and purified. To generate pAAV.A+F-EGFP, the following sequence was custom synthesized by GeneWiz and cloned into pAAV.CMV-EGFP in place of CMV (SEQ ID NO: 50):

AGAGATCATACGCGTCTAGTCATCTCAGTTGCTGTCAGCTGCTTGTG
AGCCTTCTCACATCCAGAGAATGTATCAGCATTGTGCAGACTGAAAA
GACCCAGAGGAACAAGGCTCCAATGGCAAAATTCCAAGTAGAATGAC
AAATAAATGGGGAGCCATCTGAGAGCAAGGGAGTCCTGCCCAACACC
CGCCCCATGCCTTTCTCAGGGACCTCAGACCAGCCACTCACCTCCAT
CCTCCCAGCACCACCTGCAACCAGCCCCTTGCCCTCTGCAAACTGGA
GCACGACTGGATCTTTAGATGGGGGAAAAATGCTTCATCATGTTCTG
CTGCTTCATGCAAAACCAGAAACTCCCTCCCCCTCTTCCCTCCTCCC
AGCGCACTCTCCTTCCAGTAAGTTTAAACTTCCCTCCTCGGTCCCCT
GCCCCGCCCCTCTCACTGCGGCGGAGCCGGTCGGCCGGGGGGCCGCA
GGGGAGGAGGCGGAGAGGGCGGGGCCCTCCTCCCCACCCTCTCACTG
CCAAGGGGTTGGACCCGGCCGCGGCGGCTATAAAAGGGCCGGCGCCC
TGGTGCTGCCGCAGTGCCTCCCGCCCCGTCCCGGCCTCGCGCACCTG
CTCTCACGTGATCAGAGATATCTCAGACA

pAAV.A-spacer-F-EGFP was generated by amplifying a 1866 bp section of lambda DNA using the following primers (Forward primer: 5′-ATCGATGTTTAAACTACTACCGATTCCGCCTAGT-3′ (SEQ ID NO: 51) and Reverse primer: 5′-ATGCATGTTTAAACAGGCATTTATACTCCGCTGG-3′) (SEQ ID NO: 52) and cloning this between conserved regions A and F in pAAV.A+F-EGFP. All plasmid constructs were verified by Sanger sequencing. Recombinant AAV2/2 viruses, AAV.NEFH-EGFP AAV.Nefh-EGFP, AAV.CMV-EGFP, AAV.minNefh-EGFP, AAV.A-EGFP, AAV.A+F-EGFP and AAV.A-spacer-F-EGFP were generated, and genomic titres determined, as described (O'Reilly et al., 2007).

### Animals and Intravitreal Injections

Wild type 129 S2/SvHsd mice (Harlan UK Ltd, Oxfordshire, UK) were maintained in a specific pathogen free (SPF) facility. Intravitreal injections were undertaken in strict compliance with the European Communities Regulations 2002 and 2005 (Cruelty to Animals Act) and the Association for Research in Vision and Ophthalmology (ARVO) statement for the use of animals. Adult mice were anaesthetised and pupils dilated as described (O'Reilly et al., 2007). Using topical anaesthesia (Amethocaine), a small puncture was made in the sclera. A 34-gauge blunt-ended microneedle attached to a 10 μl Hamilton syringe was inserted through the puncture, and 3 μl AAV2/2 was slowly, over a two-minute period, administered into the vitreous. Following intravitreal injection, an anesthetic reversing agent (100 mg/10 g body weight; Atipamezole Hydrochloride) was delivered by intraperitoneal injection. Body temperature was maintained using a homeothermic heating device. Animals were sacrificed by CO₂ asphyxiation.

### Histology

Histology was performed as described (Chadderton et al., 2012) with some modifications. Briefly, transduced eyes (n=6) were fixed in 4% paraformaldehyde and cryosectioned (12 μm). Sections were co-labeled for EGFP (chicken anti-GFP; Abcam, ab13970, 1/2000 dilution; Palfi et al., 2012) and either Brn3a (goat anti-Brn3a; Santa Cruz Biotechnology, sc-31984, 1/200 dilution; Nadal-Nicolás et al., 2009; Trost et al., 2015), ChAT (goat anti-choline acetyltransferase; Millipore, AB144P, 1/500 dilution; Zhu et al., 2014) or GABA (rabbit anti-GABA; Sigma, A2052, 1/2000 dilution; Zhu et al., 2014) using immunohistochemistry. EGFP was labeled with FITC-conjugated secondary antibody (1/400 dilution, Jackson ImmunoResearch Laboratories) while Brn3a, ChAT and GABA were labeled with Cy3-conjugated secondary antibody (1/400 dilution, Jackson ImmunoResearch Laboratories). Cell nuclei were counterstained with 4,6-diamidino-2-phenylindole (DAPI). Background labeling was determined using parallel processed sections where the primary antibodies were omitted. Corresponding microscope images were taken using a Zeiss Axiophot fluorescent microscope (Carl Zeiss Ltd., Welwyn Garden City, UK). Immunohistochemical signals obtained with different filters were overlaid using Photoshop v.13 (Adobe Systems Europe, Glasgow, UK). For analysis, levels for each channel were set to predetermined values to help discrimination between signal and background; signal levels above threshold were taken as positive. Additionally, cellular colocalisation of the positive immunohistochemical signals with the nuclear label was a criterion for identification of positive cells. However, it is possible that at the low spectrum identification of either positive or negative cells failed. This would have implicated a small percentage of cells and affected all groups similarly, and therefore should not have any significant effects on the results. Labeled and co-labeled cells were counted manually using the count tool in Photoshop. Two transduced sections (approximately 300 μm apart) from the central part of the retina (~1500 μm span in total) were analysed for each marker (n=4-5). Statistical analysis (one way ANOVA, Tukey's multiple comparison post-hoc test) was performed using Prism 5 (GraphPad); p<0.05 was considered statistically significant.

### Flow Cytometry Cell Sorting

Retinas were harvested three weeks post-injection and trypsin-dissociated, as previously described (Palfi et al., 2012). To isolate RGCs, cells were labeled with anti-Thy1-PE-Cy5, (CD90.2, Rat Thy-1.2, 53-2.1 1:100; eBioscience Inc., San Diego, CA). DRAQ5™ (BioStatus, Leicestershire, UK). Nucleated, DRAQ5-positive cell populations were initially sorted on the basis of forward and side scatter, and subsequently two stages of singlet selection. Retinal cells expressing both EGFP and Thy-1 were identified (BD FACSAria IIIu high speed cell sorter, BD Bioscience, San Jose, CA). EGFP had been excited by a 488 nm laser and the emission was collected using a 530/30 band pass filter. Thy-1 PECy5 had been measured exciting the probe with a 561 nm laser and collecting the signal with a 690/40 nm band pass. QC of the cell sorter had been done with BD CS&T beads and the drop delay had been adjusted using the BD Accudrop beads (RUO), following manufacture specifications. EGFP-positive cells expressing Thy-1 were represented as a percentage of the total EGFP positive cells. Data was reanalyzed with the FCSExpress 6 Flow software (DeNovo Software). Statistical analysis (Student's t-test) was performed using Microsoft Excel and p<0.05 was considered statistically significant.

### RT-QPCR of FACS Sorted Thy-1 Positive Cells

Thy1-positive cells collected from n=12 retinas and non-labelled retinal cells with a similar forward and side scatter from n=9 retinas were collected by flow cytometry cell sorting and total RNA was extracted as described (Millington-Ward et al., 2011). Thy1 mRNA was amplified in triplicate from pooled sorted populations by flow cytometry using the QuantiTect SYBR green RT-PCR kit (Qiagen, Hilden, Germany) using the manufacturer's protocol and the following primers:
F 5′ TGAACCAAAACCTTCGCCTG 3′ (SEQ ID NO: 30)
R 5′ AGCTCACAAAAGTAGTCGCC 3′ (SEQ ID NO: 31)

Resulting CT values were standardised to cell number, as standardly used housekeeping genes could be expressed at different levels in different cell populations, making them unreliable for this analysis.

### RNA Extraction and PCR Analysis

Adult wild type mice (n=5 or 6 eyes) were intravitrally injected with 6.6×10⁸ vp AAV-A-EGFP, AAV-A+F-EGFP, AAVA-spacer-F-EGFP, AAVEGFP or AAV-Nefh-EGFP. Retinas were harvested three or four weeks post-injection and total RNA extracted as described (Millington-Ward et al., 2011). In vivo expression levels of EGFP was determined by reverse transcription PCR (RT-PCR) on a StepOne Real Time PCR System (Applied Biosystems, Foster City, CA, USA) using a QuantiTect SYBR Green RT-PCR kit (Qiagen Ltd., Crawley, UK). The EGFP primers used were: EGFP forward primer 5′ TTCAAGAGGACGGCAACATCC 3′ (SEQ ID NO: 27, Table 6) and EGFP reverse primer: 5′ CACCTTGATGCCGTTCTTTCGC 3′ (SEQ ID NO: 28, Table 6). RT-PCRs were performed twice in triplicate. Expression levels were normalized using the internal housekeeping gene β-actin. Standard curves of β-actin were generated by serially diluting RNA 5×. Standard curves of EGFP were generated by serially diluting plasmid DNA containing an EGFP gene 10×. A minimum of 4 points were used in all standard curves.

### Results

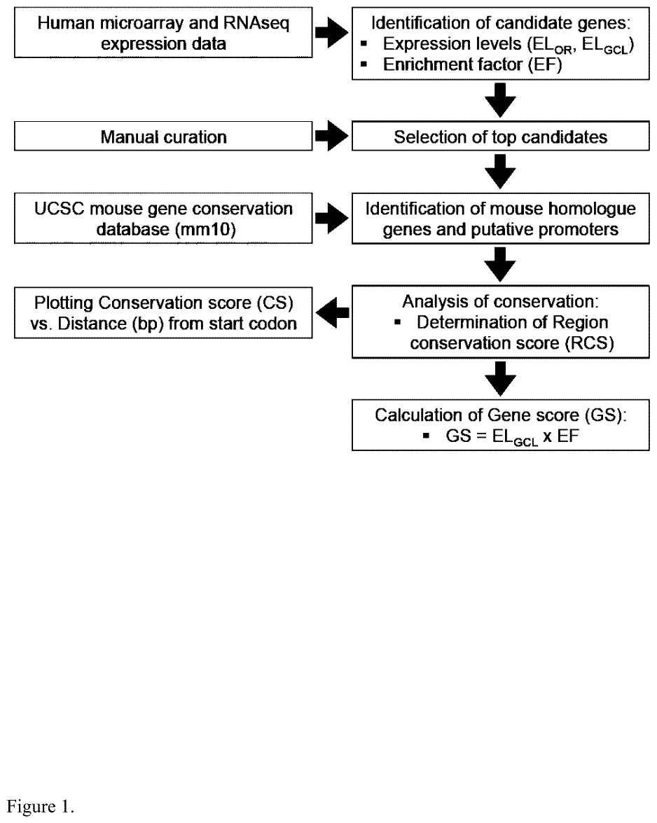

The objective of the current study was the characterisation and in vivo evaluation of an RGC promoter for future use in AAV-mediated gene therapies. A comparative evaluation of genes with highly enriched RGC expression was undertaken in silico and the lead candidate was investigated in vivo (FIG. 1). Whilst gene expression profiles of RGCs are available, the promoters that drive this expression are ill defined. We chose several key criteria to identify candidate promoters using microarray data for RGCs (Choudhury et al., 2016; Kim et al., 2006; Struebing et al., 2016). Conservation data of regions upstream of the most enriched RGC candidate genes were obtained from the UCSC genome browser database (UCSC, mm10). In the study conservation of sequence across mammals (using the mouse genome as a base) was used as a proxy for presumed function in vivo to identify putative promoters. To ensure that any promoter chosen would be suitable for future use in AAV vectors, conservation analysis was limited to the immediate 2.5 kb upstream sequence of genes. Based on the expression level of a gene in the GCL (EL_{GCL}) and the enrichment factor of that gene (EF), a gene score was generated to rank genes as candidates (GS=EL_{GCL}×EF; Table 7). The basewise species conservation in the selected upstream sequences was plotted (conservation numbered between 0 and 1) and the five genes with the highest GS are presented (FIGS. 2A and 2B).

TABLE 7
List of putative ganglion cell promoters. Human transcriptomic
data of 1,000 cell populations from RGCs versus OR (Kim et al.
2006) was used to determine relative expression levels in the
outer retina (EL_{OR}) and the GCL (EL_{GCL}). Enrichment factor
(EF) for the GCL was calculated as EF = EL_{GCL}/EL_{OR}. A
gene score (GS) was calculated as GS = (EL_{GCL} × EF)
to provide an overall score. Genes are listed in order of GS.

| Rank | Gene name | ELGCL | ELR | EF | GS |
|---|---|---|---|---|---|
| 1 | NEFH | 21899.1 | 89.4 | 245 | 5.37 × 10⁶ |
| 2 | NEFM | 6984.1 | 31.7 | 220.6 | 1.54 × 10⁶ |
| 3 | NEFL | 7841.1 | 50.5 | 155.3 | 1.22 × 10⁶ |
| 4 | VSNL1 | 4659.33 | 67.35 | 69.18 | 3.22 × 10⁵ |
| 5 | SPARCL1 | 5077 | 149.75 | 33.9 | 1.72 × 10⁵ |
| 6 | SLC17A6 | 1302.9 | 10.3 | 126.8 | 1.65 × 10⁵ |
| 7 | TMSB10 | 7124.3 | 324.6 | 21.9 | 1.56 × 10⁵ |
| 8 | ANXA2 | 2221.4 | 37.5 | 59.3 | 1.32 × 10⁵ |
| 9 | STMN2 | 4139.9 | 147.9 | 28 | 1.16 × 10⁵ |
| 10 | PRPH1 | 1238.5 | 18.4 | 67.5 | 8.36 × 10⁴ |
| 11 | CRTAC1 | 4478.6 | 347 | 12.9 | 5.78 × 10⁴ |
| 12 | RBPMS | 832.5 | 12.6 | 66 | 5.49 × 10⁴ |
| 13 | RAB13 | 1802.7 | 59.7 | 30.2 | 5.44 × 10⁴ |
| 14 | ATP1B1 | 3803.3 | 299.2 | 12.7 | 4.83 × 10⁴ |
| 15 | FABP3 | 1054.6 | 24.9 | 42.4 | 4.47 × 10⁴ |

Following analysis, Nefh was deemed to be the most highly enriched gene in RGCs with an enrichment factor (EF) of 245-fold, as well as demonstrating an extremely high EL_{GCL} (21899.1; Table 7). Some of the mouse genes analysed showed greater average conservation in their 2.5 kb upstream regions than Nefh (Nefm 0.289, Stmn2 0.292, Crtac1 0.349 vs. Nefh 0.185). However, due to their lower EF and EL_{GCL} scores, Nefh was deemed likely to drive higher levels of RGC-specific expression and hence to be a better candidate promoter (GS: Nefh 5.37×10⁶ vs. Nefm 1.54×10⁶, Stmn2 1.16×10⁶, Crtac1 5.78×10⁴). Tmsb10, Nefl, and Sparcl1 had lower scores than Nefh in all categories. Brn3a, a commonly used marker for RGCs (Kim et al., 2006; Nadal-Nicolás et al., 2014), was found to have an extremely high conservation within a 2.5 kb upstream region, and a high EF (0.576, 79.1 respectively). However, its EL_{GCL} was found to be approximately 39 times lower than that of Nefh (719.7), and so was not included as a candidate gene. The hSYN gene showed no significant GCL enrichment or expression in the Kim et al (2006) study.

To explore the strength and specificity of the putative Nefh promoter, 2251 bp of upstream sequence from the mouse homologue was used to drive expression of an EGFP reporter gene in an AAV2/2 vector (AAV-Nefh-EGFP) and expression compared to that mediated by the CMV promoter (AAV-CMV-EGFP; Palfi et al., 2010, Chadderton et al., 2012). The mouse gene was chosen to ensure that function or non-function was not due to species incompatibility. The CMV promoter incorporated into AAV vectors has previously been shown to drive high levels of transgene expression in a wide variety of retinal cell types (Lebherz et al., 2008; Li et al., 2008; Mueller and Flotte, 2008), including RGCs (Chadderton et al., 2012; Tshilenge et al., 2016) and was used as a control vector for transgene expression.

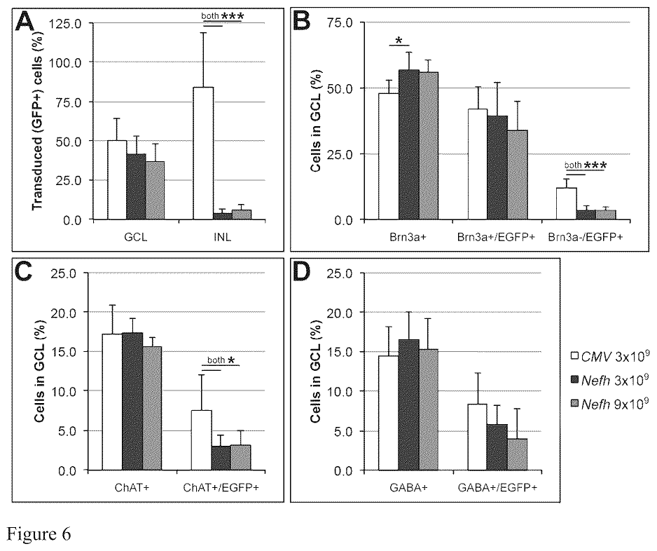

Adult mice were injected intravitreally with 3×10⁹ viral genomes (vg)/eye AAV.CMV-EGFP or with either 3×10⁹ vg/eye or 9×10⁹ vg/eye AAV.Nefh-EGFP. Histological analysis 12 weeks post-injection revealed widespread EGFP expression in the retina (FIGS. 4A-4F). Individual cells exhibited a broad range of EGFP expression levels from low to very high, possibly due to varying viral transduction. However, cellular EGFP labeling (colocalised to DAPI stained nuclei), even for cells expressing low levels of EGFP, was easily distinguishable from uniform background levels. EGFP expression from both promoters was observed in a significant number of cells in the GCL (50.2±14.1% AAV.CMV-EGFP, FIGS. 4A and 4D; 42±11.2% AAV.Nefh-EGFP, FIGS. 4-B and 4E; and 37±11.1% high dose AAV.Nefh-EGFP, FIGS. 4C and 4F and FIG. 6A). However, while the Nefh promoter mediated EGFP expression was predominantly confined to the GCL (FIGS. 4B, 4E, 4C and 4F), CMV promoter driven expression extended into the INL (FIGS. 4A and 4D); 84.5±34.2% AAV.CMV-EGFP, 3.6±2.9% AAV.Nefh-EGFP and 5.6±3.8% high dose AAV.Nefh-EGFP (FIG. 6A; EGFP-positive cells in the INL expressed as a percentage of all cells in the GCL). Notably, the increased dose of AAV.Nefh-EGFP did not increase the transduction rate in the INL (FIGS. 4A-4F and FIG. 6A). AAV.CMV-EGFP demonstrated significantly greater INL expression compared to AAV.Nefh-EGFP, p<0.001 (FIGS. 4A-4F and FIG. 6A).

Approximately fifty percent of cells in the GCL are RGCs with the other fifty percent being displaced amacrine cells (Akopian et al., 2016; Jeon et al., 1998; webvision.med.utah.edu). To further delineate the expression profile of the Nefh promoter, EGFP transgene expression was analysed in the GCL using antibodies targeting Brn3a, an RGC marker (Schlamp et al., 2013) and two amacrine cell markers, ChAT and GABA (Jeon et al., 1998; Wässle et al., 1987; webvision.med.utah.edu). Brn3a staining was used to explore the specificity of the Nefh promoter for RGCs; 50%-55% of all cells in the GCL were Brn3a positive in line with previously published data (FIGS. 5A-5O and FIG. 6B; Schlamp et al., 2013, Jeon et al., 1998). FIGS. 5A-5O display representative staining of the 3×10⁹ vg/eye dose of AAV.Nefh-EGFP. While AAV.CMV-EGFP and AAV.Nefh-EGFP expressed in comparable numbers of Brn3a positive cells (41.9±8.5% AAV.CMV-EGFP, 39.5±12.7% AAV.Nefh-EGFP and 33.9±11.2% high dose AAV.Nefh-EGFP; FIGS. 5A-5O and FIG. 6B), Nefh promoter-mediated EGFP expression in the GCL was observed in significantly fewer Brn3a negative cells (p<0.001, 12.1±3.3 AAV.CMV-EGFP, 3.5±1.7 AAV.Nefh-EGFP and 3.4±1.2 high dose AAV.Nefh-EGFP; FIGS. 5A-5O and FIG. 6B). ChAT and GABA markers were used to identify subpopulations of amacrine cells; identifying 17% and approximately 15% of cells in the GCL, respectively, in the mouse retina (FIGS. 5A-5O, FIGS. 6C and 6D). EGFP expressing cells were significantly more likely to be ChAT positive amacrine cells when EGFP expression was driven by the CMV promoter, compared to the Nefh promoter (p<0.05, 7.5±4.6% AAV.CMV-EGFP, 3.1±1.4% AAV.Nefh-EGFP and 3.2±1.9% high dose AAV.Nefh-EGFP; FIGS. 5A-5O and FIG. 6C). Additionally a greater number of CMV promoter driven EGFP positive cells were also co-labeled with GABA, however this represented a trend rather than reaching significance (8.3±4.0% AAV.CMV-EGFP, 5.8±2.4% AAV.Nefh-EGFP and 4.0±3.7% high dose AAV.Nefh-EGFP; FIGS. 5A-5O and FIG. 6D).

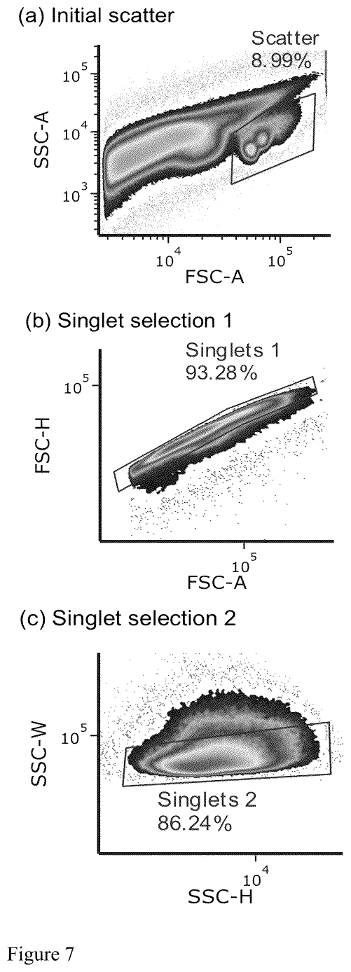

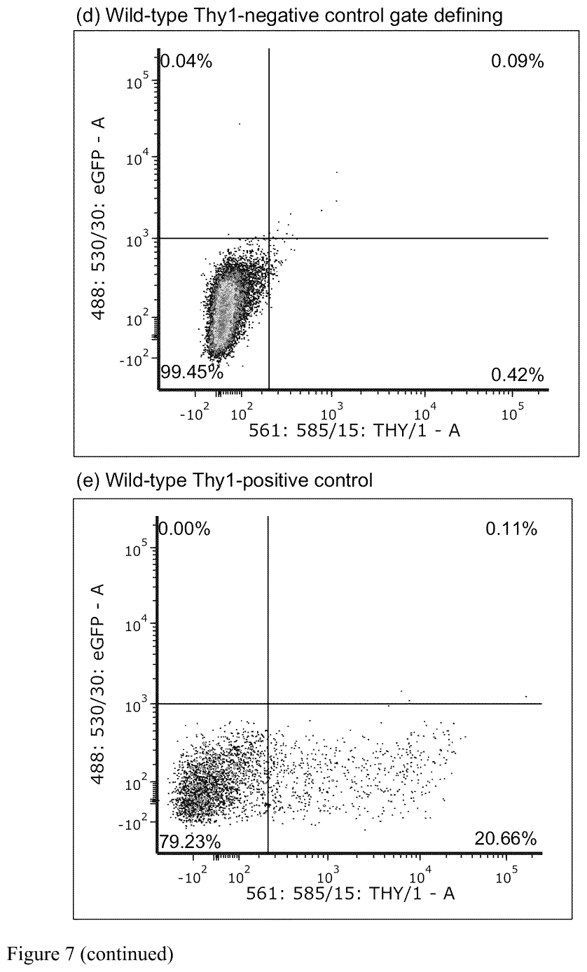

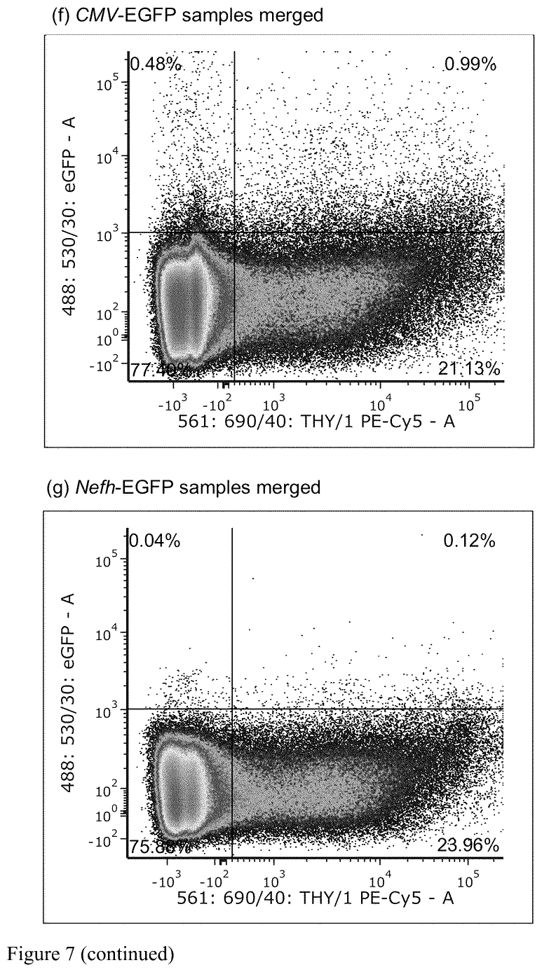

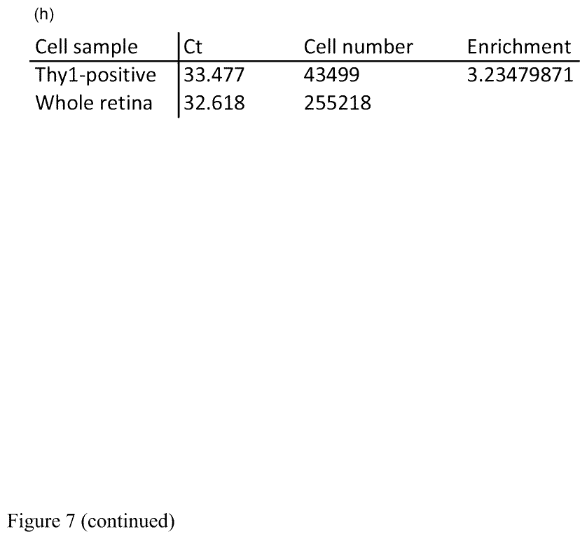

As a second method of assessing preferential gene expression in RGCs from the Nefh promoter, adult wildtype mice were intravitreally injected with 9×10⁹ vg/eye AAV.Nefh-EGFP or 3×10⁹ vg/eye AAV.CMV-EGFP. Three weeks post injection, retinas were taken, cells dissociated and analysed by FACS and EGFP-positive cells assessed for Thy1 expression. Interestingly levels of Thy1 enrichment in these populations were significantly higher in AAV.Nefh-EGFP versus AAV.CMV-EGFP transduced retinal samples (5.4-fold, n=12 versus only 1.6-fold, n=9 respectively; p<0.005). These data support the immunohistochemical observations above. Notably, Thy1 mRNA levels were found to be 3.23-fold higher in Thy1-positive cells than in non-antibody labeled retinal cells with a similar forward and sideways scatter (CT values of 32.618 and 33.477 respectively), indicating that the Thy1 antibody enriches for RGCs (FIGS. 7A-7H).

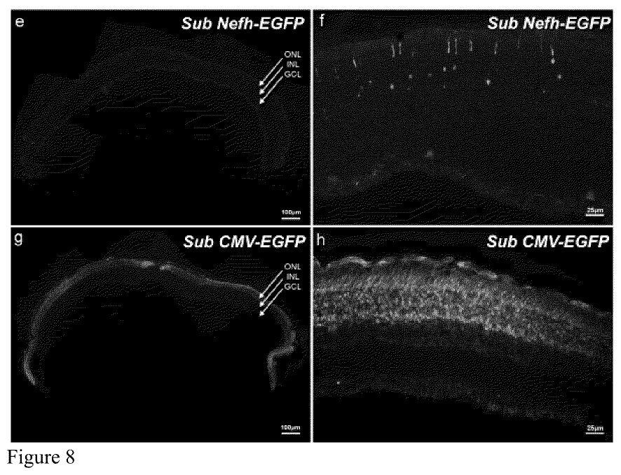

To further explore the preferential gene expression in RGCs from the Nefh promoter, adult wildtype mice were subretinally injected with 3×10⁹ vg/eye AAV.Nefh-EGFP or AAV.CMV-EGFP and EGFP expression evaluated. The AAV2 serotype efficiently transduces RGCs; however, prior studies have shown that it will not transduce photoreceptors when injected intravitreally. As such, it was necessary to confirm an absence of transgene expression in photoreceptors when AAV.Nefh-EGFP was administered subretinally (FIG. 8). Subretinal AAV.CMV-EGFP shows wide expression, with near total transfection of the photoreceptor layer. Comparatively, AAV.Nefh-EGFP shows almost no expression when administered subretinally.

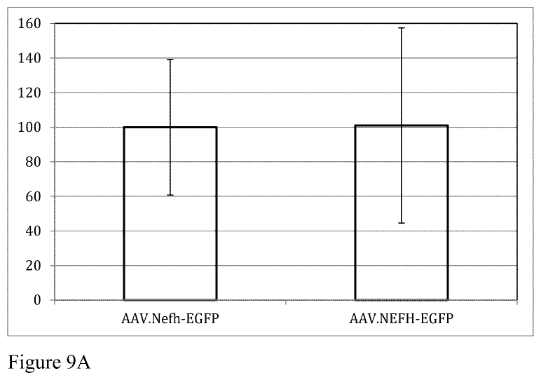

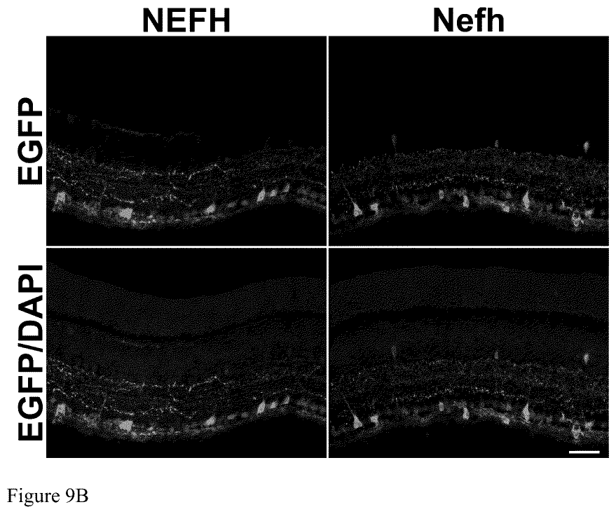

Given the potential demonstrated by 2251 bp of upstream sequence of the Nefh gene to preferentially transduce RGCs in the murine experiments above, it was important to evaluate the putative promoter region from NEFH. 2501 bp of upstream sequence from the human gene was used to drive expression of an EGFP reporter gene in an AAV2/2 vector (AAV-NEFH-EGFP) and expression compared to that mediated by the murine Nefh promoter (AAV-Nefh-EGFP; FIGS. 9A and 9B). Adult mice were injected intravitreally with 6.6×10⁸ viral genomes (vg)/eye AAV.NEFH-EGFP or 6.6×10⁸ vg/eye AAV.Nefh-EGFP. Histological analysis three weeks post-injection revealed AAV-NEFH-EGFP mediated EGFP expression to be predominantly confined to the GCL in the retina (FIG. 9B) in a similar manner to AAV-Nefh-EGFP. Native EGFP fluorescence was present in both AAV.NEFH-EGFP and AAV-Nefh-EGFP transduced retinas; Cy5-labeled immunostaining enhanced detection of EGFP. Both native and immunostained EGFP signals were similar in AAV.NEFH-EGFP and AAV-Nefh-EGFP transduced retinas. Cells in the GCL, as well as, dendrites in the IPL were detected. A few cells were also labeled in the INL mostly at the INL/IPL boundary; minimal label was present in the OPL. Individual cells exhibited a broad range of EGFP expression levels from low to very high, possibly due to varying viral transduction. However, cellular EGFP labeling (colocalised to DAPI stained nuclei), even for cells expressing low levels of EGFP, was easily distinguishable from uniform background levels. EGFP expression from NEFH was observed in a similarly significant number of RGCs as Nefh.

Similarly EGFP RNA expression levels from AAV-Nefh-EGFP and AAV-NEFH-EGFP were compared in wild type mice. Mice were injected intravitreally with 6.6×10⁸ vp of either vectors and retinas taken 3 weeks post-injection. EGFP RNA levels expressed from both vectors did not differ significantly and in this in vivo study were shown to be functionally equivalent (FIG. 9A).

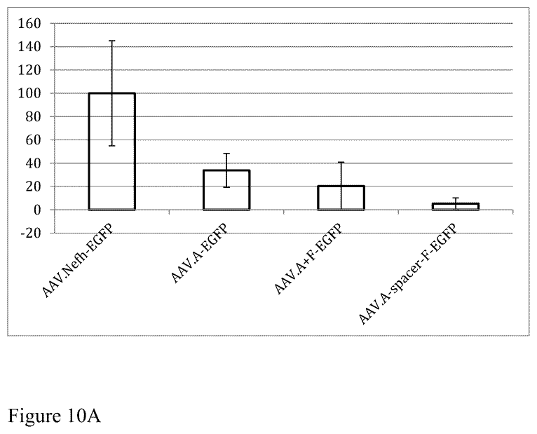

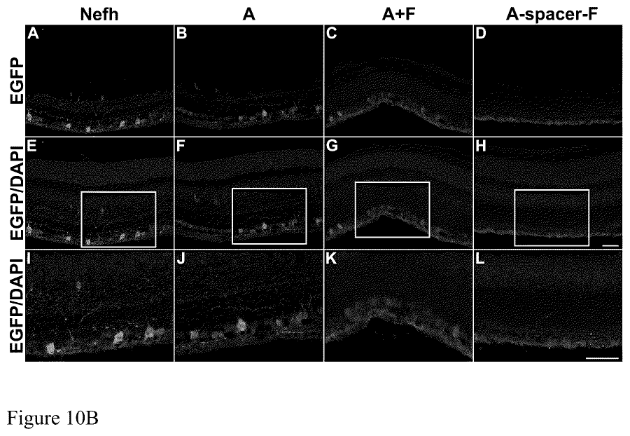

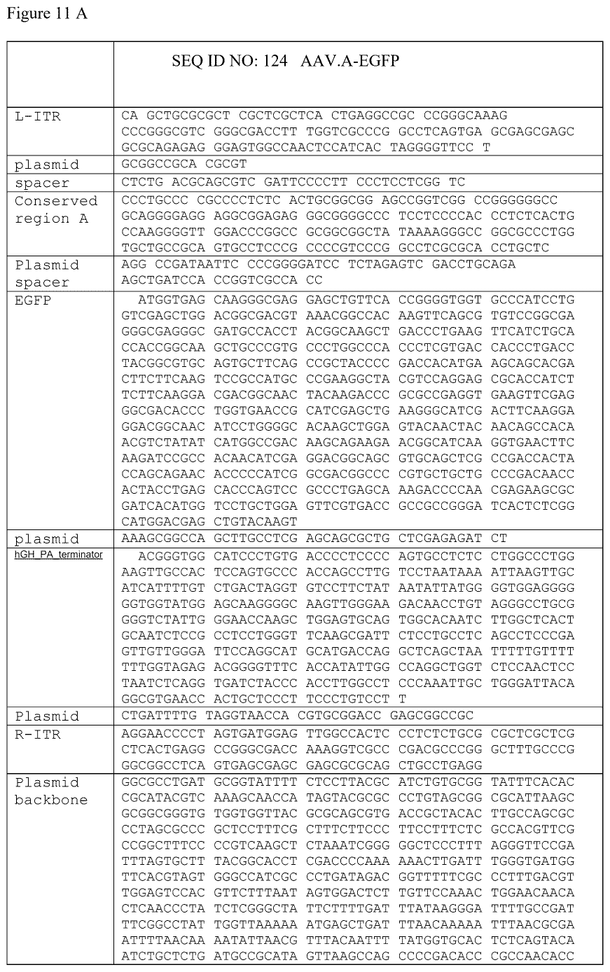

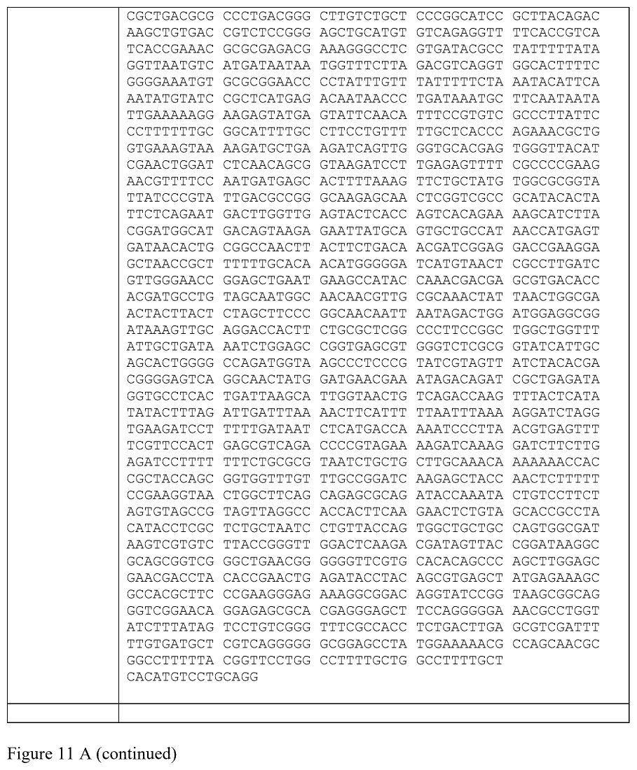

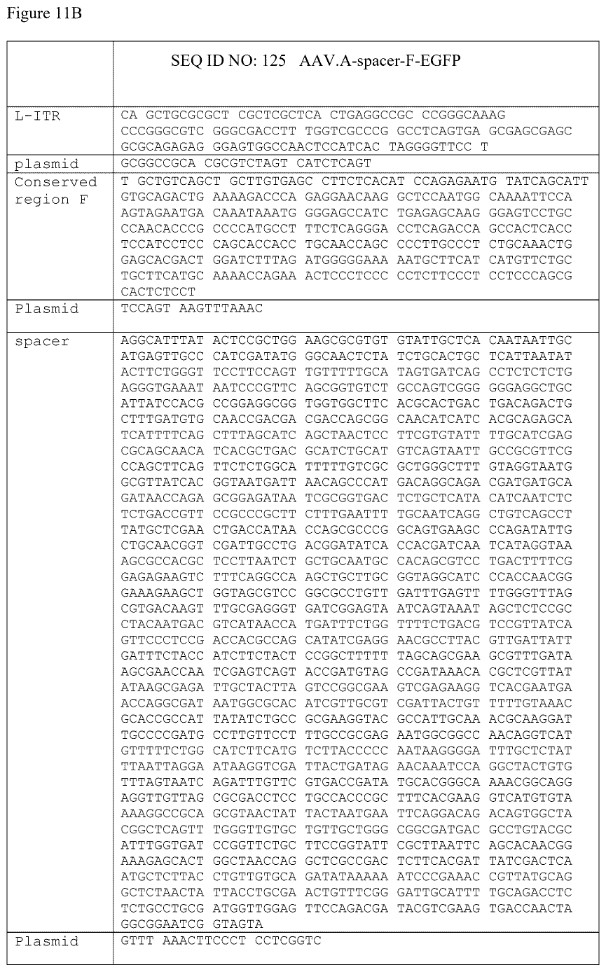

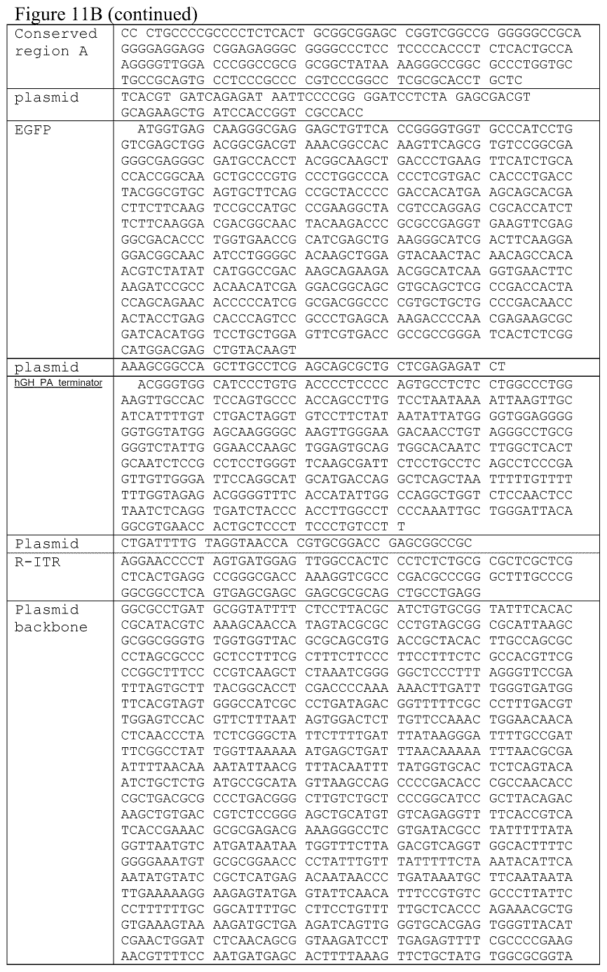

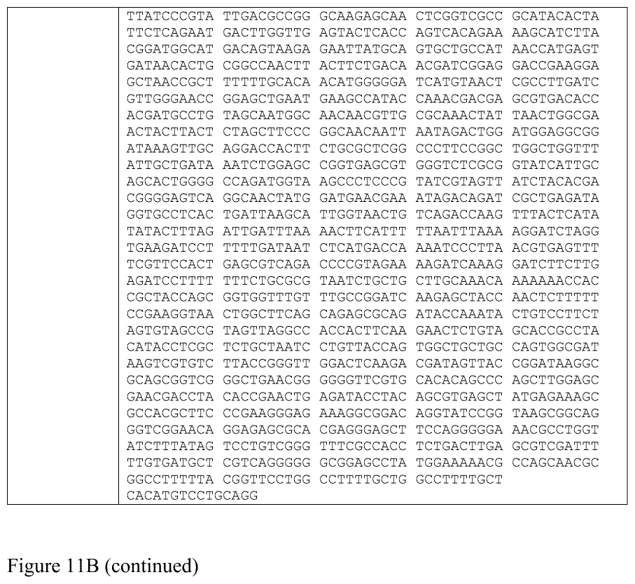

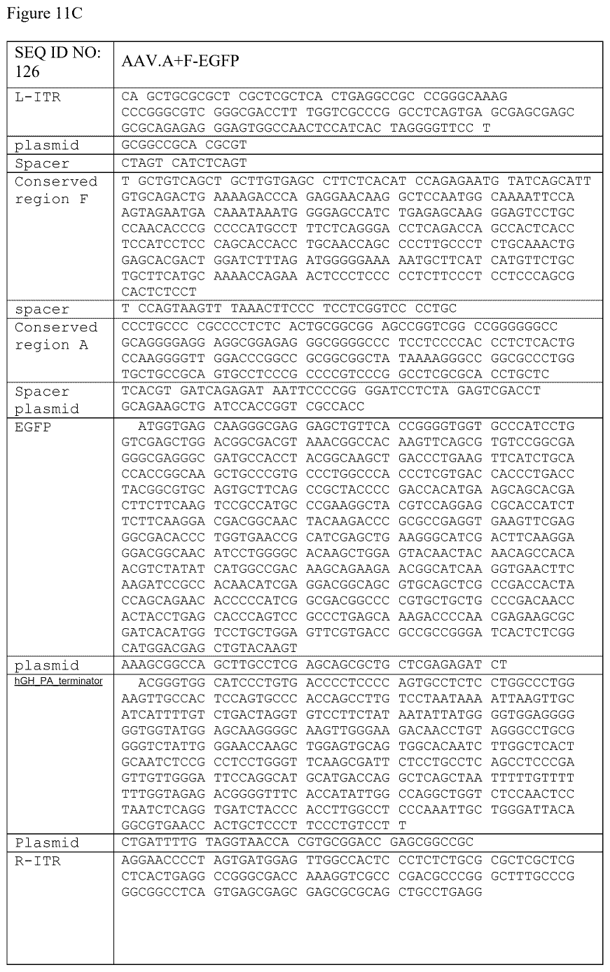

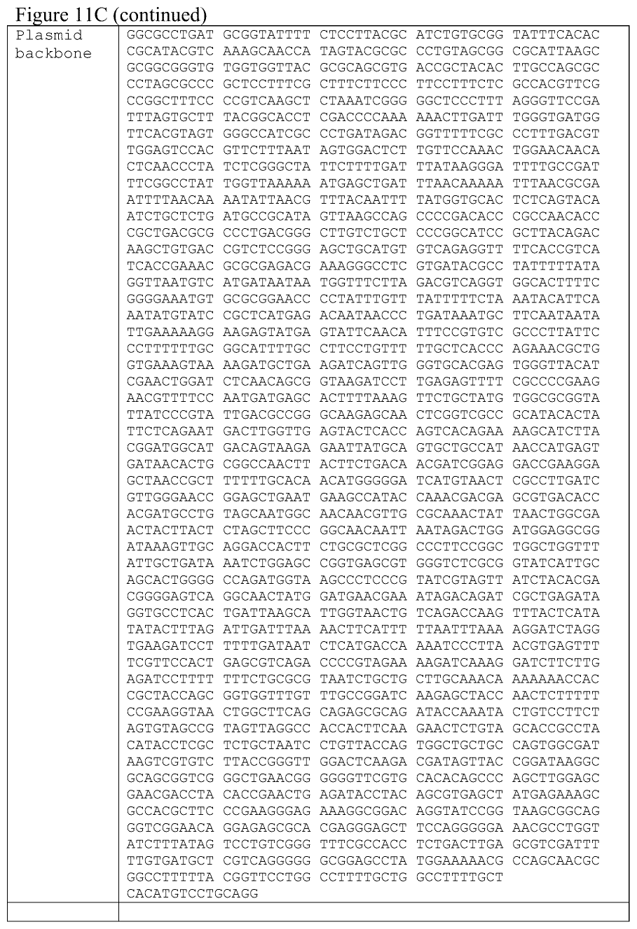

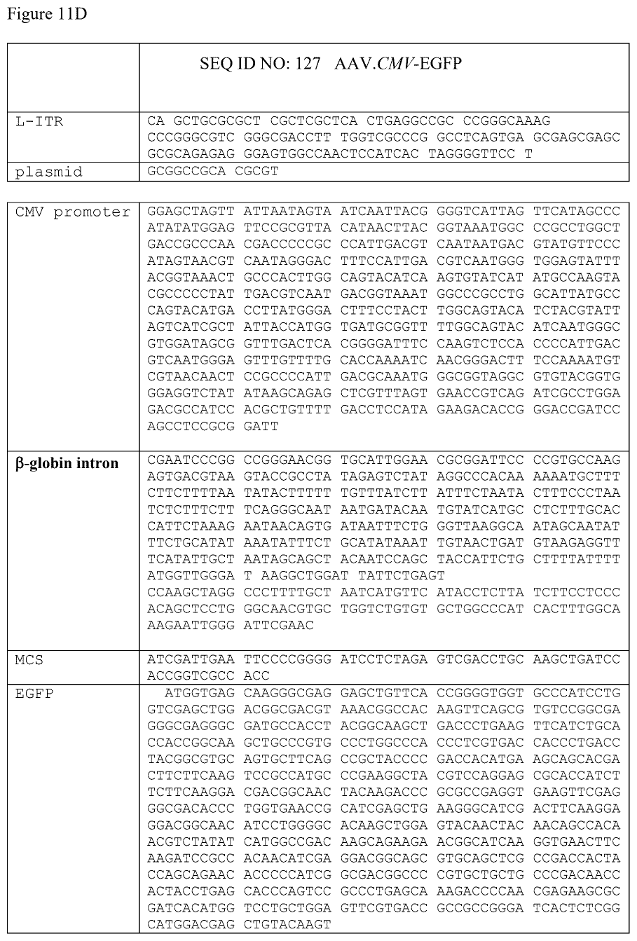

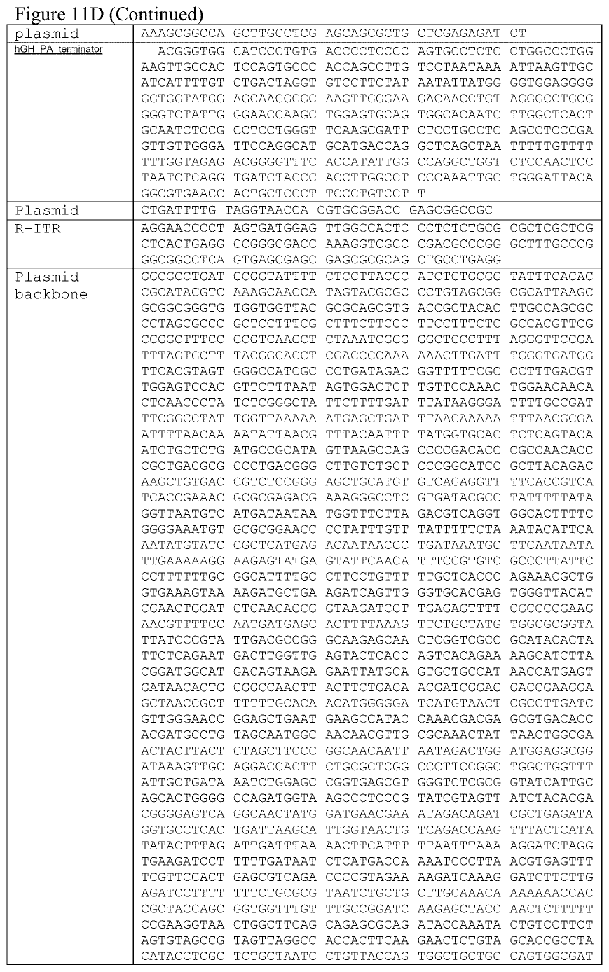

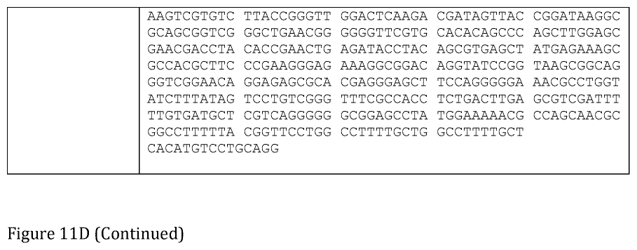

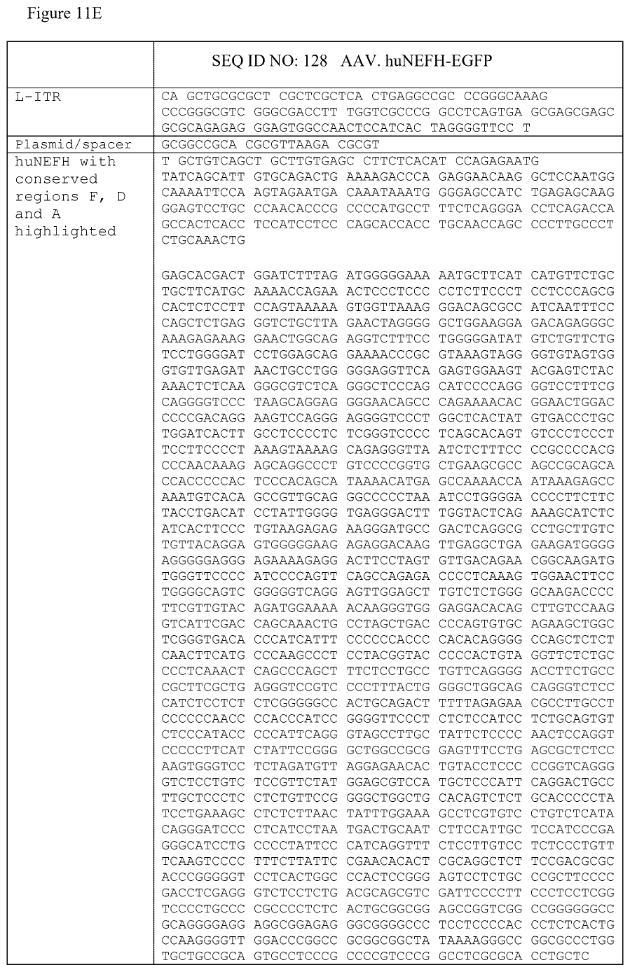

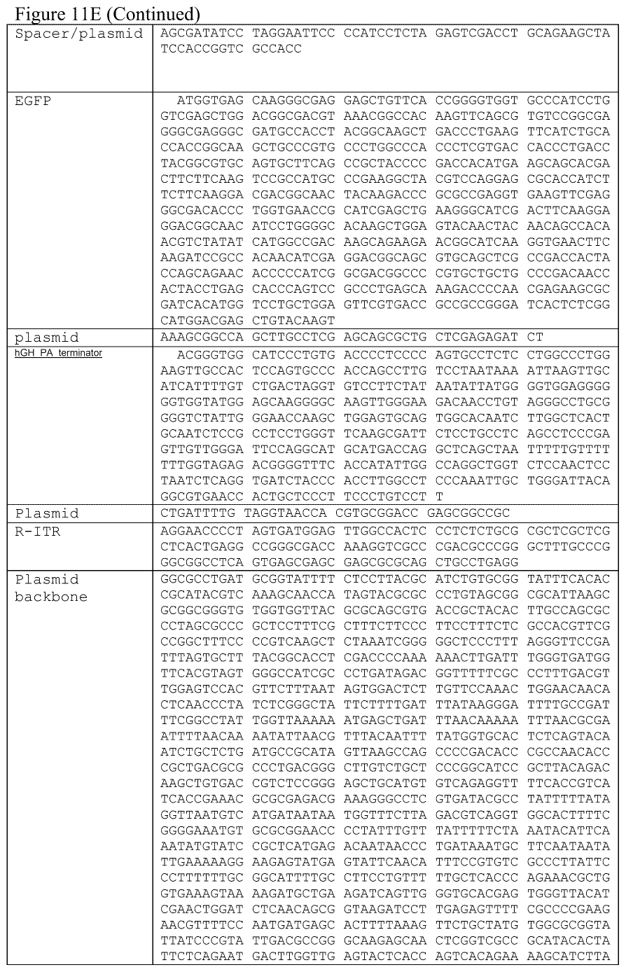

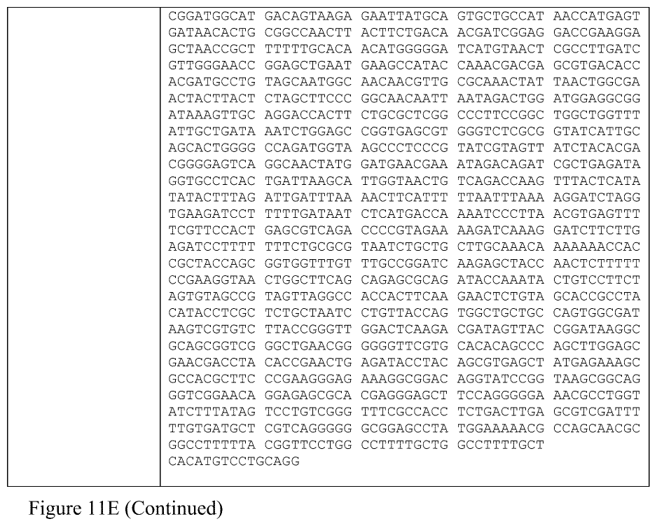

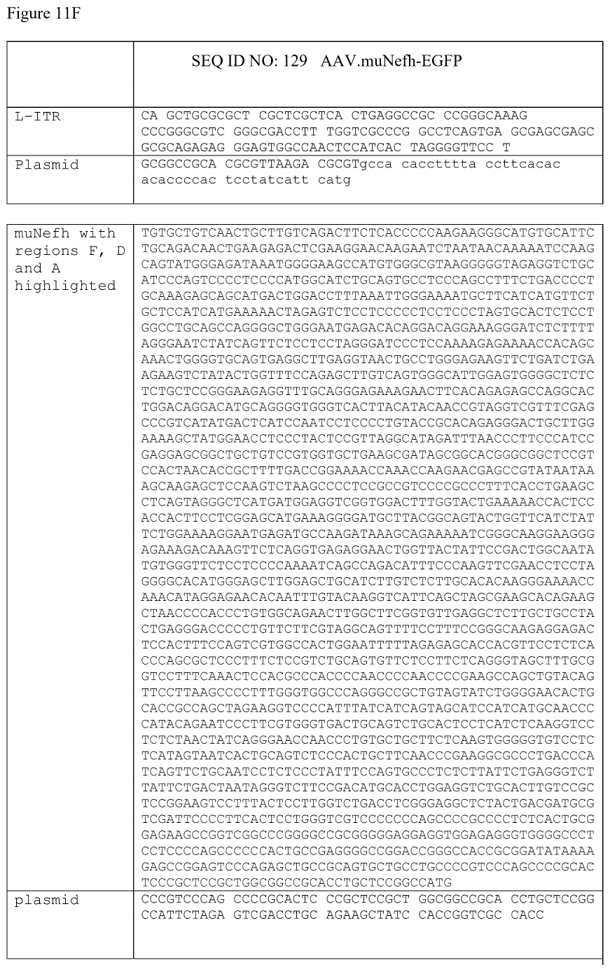

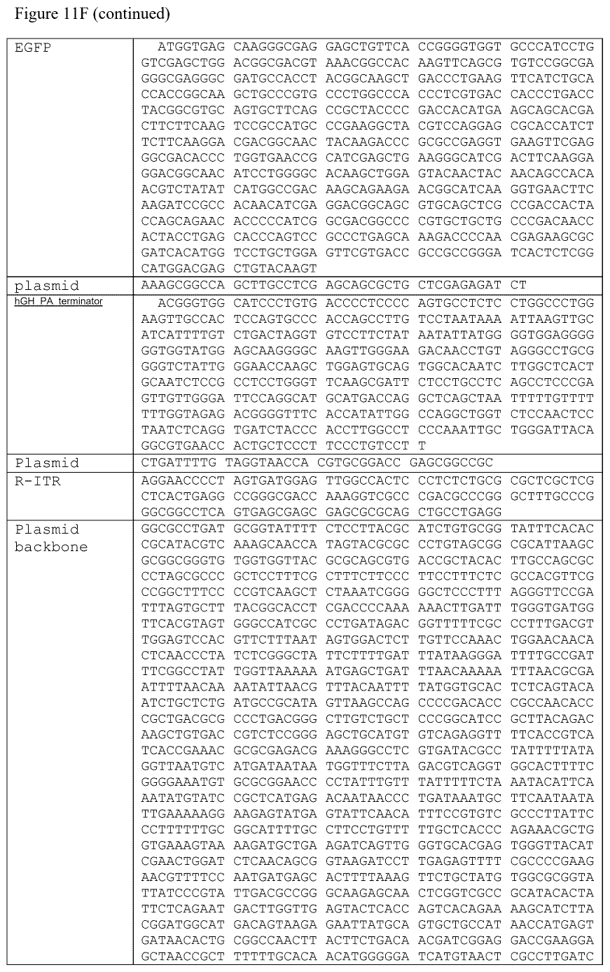

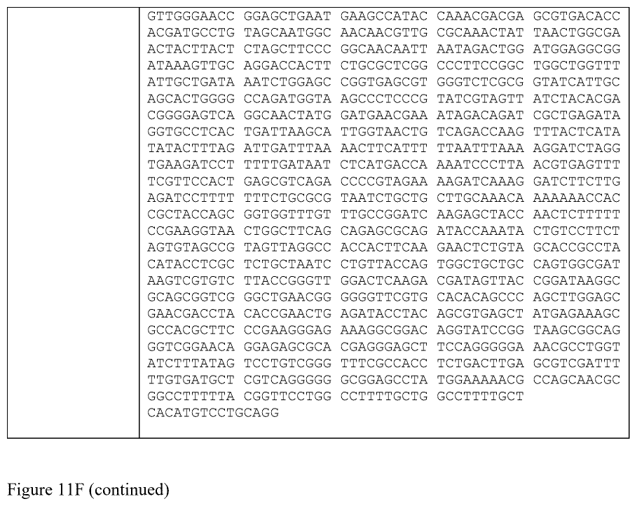

To further explore the individually defined elements of the putative NEFH promoter a series of constructs were generated (FIG. 2B). AAV-A-EGFP contains a single conserved upstream region (SEQ ID NO: 1) to drive expression of an EGFP reporter gene. AAV-A+F-EGFP utilises A (defined above) plus F (SEQ ID NO: 3) to drive expression of an EGFP reporter gene. AAV-A-spacer-F-EGFP utilises A and F (as defined) separated by spacer DNA (SEQ ID NO: 24), in this instance, to drive expression of an EGFP reporter gene. The inclusion of the spacer mimics the natural spacing of the two elements (A and F) within the NEFH upstream region. Adult mice were injected intravitreally with 6.6×10⁸ viral genomes (vg)/eye of either construct or with 6.6×10⁸ vg/eye AAV.Nefh-EGFP. Histological and EGFP RNA expression analyses 4 weeks post-injection were performed (FIGS. 10A and 10B).

The constructs evaluated expressed EGFP at varying levels, with AAV.Nefh-EGFP expressing significantly more highly than any of the other constructs (p<0.05; FIG. 10A). EGFP expression in transduced retinas was analysed four weeks post-injection (n=4). Native EGFP fluorescence was present in AAV.Nefh-EGFP, and AAV.A-EGFP transduced retinas; very faint label was detected in AAV.A+F-EGFP transduced retinas, while no specific label was present in AAV.A-spacer-F-EGFP transduced retinas (FIG. 10B; native EGFP fluorescence not shown in figure). Cy5-labeled immunostaining enhanced detection of EGFP (there was no immunolabel in AAV.A-spacer-F-EGFP transduced retinas). Cells in the GCL were labeled as well as dendrites in the IPL in AAV.Nefh-EGFP and AAV.A-EGFP treated retinas. A few cells were also labeled in the INL, mostly at the INL/IPL boundary. Only cell bodies without dendrites were detected in AAV.A+F-EGFP treated retinas. EGFP expression was strongest in AAV.Nefh-EGFP, followed by AAV.A-EGFP and AAV.A+F-EGFP, respectively.

### Discussion

AAV has become one of the commonly used vectors for gene therapy, with many clinical trials ongoing or completed and a number of gene therapies approved or seeking approval (clinicaltrials.gov). AAV is the dominant vector for use in ocular gene therapies (Bainbridge et al., 2015; Bennett et al., 2016; Feuer et al., 2016; Ghazi et al., 2016; Hauswirth et al., 2008; MacLaren et al., 2014; clinicaltrials.gov), and research in recent years has focused on improving the efficiency of AAV transduction and expression in the retina. The development of AAV vectors such as AAV7m8 and AAV8BP2 has improved levels of transduction in a wide variety of retinal cell types, and enabled consideration of intravitreal administration as a potential route of access for many retinal cells including photoreceptors (Cronin et al., 2014; Dalkara et al., 2013; Ramachandran et al., 2016). Various tyrosine capsid mutations in AAV have the potential to increase transgene expression levels by modulating capsid phosphorylation and ubiquitin proteasome-based degradation of viral particles during intracellular trafficking (Mao et al., 2016; Mowat et al., 2014; Petrs-Silva et al., 2009). Recent approaches to intravitreal delivery, including vitrectomy and sub-inner limiting membrane (sub-ILM) blebbing, have the potential to improve expression levels further (Boye et al., 2010; Tshilenge et al., 2016). However, a consequence of more efficient and broad transduction profiles may be greater potential for off-target effects. Confining expression of a gene therapy to only those cells affected by a disease represents a rational strategy; the potential reduction in immune responses may be an advantageous safety feature, as well as a means of aiding long-term expression.

In the current study, we have developed an approach to identify putative RGC promoters by analysing retinal transcriptomic data and referencing it against mammalian sequence conservation datasets to infer potential function. The expression levels of retinal genes were analysed, with high GCL enrichment and high absolute expression levels prioritised. Gene expression data in RGCs from the gene expression omnibus (GEO; ncbi.nlm.nih.gov/geo) was analysed in detail. Studies on expression from pre-natal or immature retina were omitted. In addition, samples where photoreceptor cell-specific gene expression was found to be high in RGCs were excluded as this indicated sample impurity. In contrast to the data from Kim et al. (2006), and taking the above into account, no studies in the database suitably provided data on RGC gene expression enrichment in adult retina.

Conservation of the upstream sequence of these genes was evaluated in this context in order to establish lead candidate promoter sequences. Using this approach, we identified a number of potential promoters for use in RGCs. We proceeded to evaluate in vivo one of these, Nefh, a putative promoter sequence that showed significant conservation between species, high retina expression and RGC enrichment and that was of a suitable size for use in AAV-mediated gene delivery vectors. We established that the Nefh upstream sequence efficiently drives expression in RGCs following intravitreal injection of AAV.Nefh-EGFP.

Following intravitreal delivery of either AAV.Nefh-EGFP or AAV.CMV-EGFP, EGFP expression patterns were compared by histology. Serotype AAV2/2 was chosen both for its efficient transduction of mouse RGCs, as well as its use and tolerance in the human eye, as has been observed in several clinical trials (Bennett et al., 2016; Busskamp et al., 2010; Ghazi et al., 2016; Koilkonda et al., 2014; MacLaren et al., 2014; Sengupta et al., 2016; Yang et al., 2016; Zhang et al., 2009). Both the Nefh and CMV promoters drove effective expression of EGFP in the GCL (FIGS. 4A-4F). Of note, the AAV.CMV-EGFP vector also resulted in expression in the INL, while AAV.Nefh-EGFP expression was predominantly confined to the GCL, with few EGFP positive cells observed in the INL (FIGS. 4A-4F and FIG. 6A). Furthermore, when an increased dose of the AAV.Nefh-EGFP vector was administered, the levels of EGFP expression in the INL did not increase, highlighting the relative specificity of the Nefh promoter compared to CMV.

Fifty percent of the GCL is composed of amacrine cells (Akopian et al., 2016; Jeon et al., 1998; webvision.med.utah.edu). Analysis of EGFP expression in Brn3a-negative cells, as well as in GABA-positive or ChAT-positive amacrine cells, two major types of amacrine cells in the mouse GCL, demonstrated that AAV.Nefh-EGFP resulted in transgene expression in significantly fewer amacrine cells compared to AAV.CMV-EGFP. While expression from the Nefh promoter was significantly restricted to ChAT-positive amacrine cells in the GCL compared to the CMV promoter, expression from both promoters were similar for GABA expressing amacrine cells in the GCL. This further highlights the relative specificity of the Nefh promoter sequence in targeting RGCs, and underlines its potential use for gene delivery to RGCs and its value for future gene therapies directed towards the retinal GCL. Of note, no significant difference was found between the numbers of transduced RGCs between the two doses of AAV.Nefh-EGFP. Previous studies have shown that only 40-60% of cells in the GCL are actually RGCs (Schlamp et al., 2013; Xiang et al., 1996); it may be that saturation of RGC transduction is being reached even at the lower AAV.Nefh-EGFP dose.

RGCs represent a heterogeneous population thought to comprise in the region of 30 discrete types, which together represent just approximately 1% of cells in the retina (Baden et al. 2016). This has made isolation of pure populations of RGCs highly challenging within the field. Methods that have traditionally been used to enrich for RGC, commonly using the Thy1 antibody, have included immunopanning (Barres et al., 1988; Welsbie et al., 2017), density gradient centrifugation (Kornguth et al., 1981), and magnetic cell separation (Shoge et al., 1999). More recently flow cytometry based methods with the Thy1.2 antibody have been used for RGC enrichment (Chintalapudi et al., 2016). These studies have highlighted that while the Thy1 antibody does indeed enrich for RGCs it does not exclusively label these cells, indicating that RGC-isolation methodologies still require optimisation. In the current study we used Thy1.2-based flow cytometry to support the data from immunohistochemistry. Similar to other studies, we found that the antibody did not exclusively isolate RGCs, based on the percentage of Thy1-positive cells. However, in addition we confirmed at the RNA level that Thy1 was enriched in our cell-sorted population. We found the enrichment of Thy1-positive cells within the EGFP-positive cell population to be greater in AAV.Nefh-EGFP versus AAV.CMV-EGFP treated retinal cell samples confirming the histological data, indicating preferential gene expression in RGCs with the Nefh promoter.

To expand the potential of the identified Nefh promoter to future human studies we also tested 2501 bp of the putative promoter region upstream of NEFH and demonstrated comparable levels of both expression and specificity in the mouse retina (FIGS. 9A and 9B). Furthermore, when a series of constructs containing one or more of the conserved regions were evaluated in vivo we found that conserved region A alone (SEQ ID NO: 1) resulted in strong EGFP expression preferentially within RGCs (FIGS. 10A and 10B). When conserved region A was combined with conserved region F the level of EGFP expression was reduced. Notably, EGFP expression was reduced when the natural spacing of the two conserved regions was mimicked with a stuffer fragment. Given the size limitations of AAV, the identification of a small region that drives strong and preferential expression in RGCs is highly important to the field of retinal gene therapy and others fields requiring RGC expression.

The purpose of this study was two-fold, involving identification of candidate RGC promoters for potential use in AAV-mediated gene therapies, and moreover the validation of the utilised methodology for characterisation of putative promoter sequences (FIG. 1, FIG. 2A). As sequencing costs continue to decrease and techniques such as RNAseq become more widely adopted, access to transcriptomic datasets from a wide variety of cell types will become more readily available. The availability of such large datasets will be a powerful resource, which, in a similar fashion to the present work, could be exploited to identify, characterise and validate promoter sequences. The current study utilised an AAV2/2 vector to facilitate the transduction of mouse RGCs. However, it has been previously observed that, while AAV2/2 is well tolerated in the human eye when administered subretinally, its transduction efficiency in primate RGCs is inferior to that of mice (Ivanova et al., 2010; Tshilenge et al., 2016; Yin et al., 2011). The development of new capsid serotypes such as AAV8BP2 (Ramachandran et al., 2016) amongst others, or new methods of administering AAV2/2 (as in the sub-ILM delivery of Boye et al., 2016) should aid in addressing this.

Intravitreal injection represents a route of vector administration that enables efficient transduction of RGCs. RGCs are the primary target cell population for gene therapies for many disorders including Leber Hereditary Optic Neuropathy (LHON), dominant optic atrophy (DOA), glaucoma and the retinal endophenotypes that are a feature of many neurodegenerative disorders, such as multiple sclerosis (Farrar et al., 2013). While intravitreal administration provides access to RGCs, it may more readily result in stimulating immune response(s) to vectors such as AAV compared to subretinal administration (Li et al., 2008). It would therefore be valuable to minimise the therapeutic vector dose, and to confine transgene expression to the target cells of interest, thereby limiting undesired side effects.

Furthermore, observations regarding patterns of cellular loss in end stage photoreceptor degenerations have highlighted the retention of certain retinal layers. While frequently the photoreceptor layer degenerates, many other retinal cells remain relatively intact, including bipolar, amacrine, horizontal and RGCs. These observations have been elegantly juxtaposed with the identification of light sensitive molecules from organisms such as algae and archaebacteria. Optogenetics is the expression of these molecules, provided as a gene therapy or protein, in non-light sensitive neurons thereby introducing a capacity for light detection. RGCs represent one key target cell population for optogenetics (Farrar et al., 2014; Gaub et al., 2014), and hence the NEFH promoter characterised in the current study, in principle, may also be of value in the design of future optogenetic-based gene therapies for IRDs. The above highlights the potential utility of the NEFH promoter sequence identified in the current study providing preferential transgene expression in RGCs in the design of future gene therapies for many disorders involving RGCs.

TABLE 8
List of animal sequences used for conservation alignment. A placental
mammal species alignment (phastConsElements60way EuarchontoGlires)
was used for the conservation alignment seen in FIGs. 2A-2B. Species
are grouped as Glires, Primates, and other placental mammals, with
species names, sequence assembly dates, and assembly details listed.

| | | Assembly | |
|---|---|---|---|
| Animal | Species | Date | Assembly Name/details |
| Mouse | Mus musculus | December 2011 | GRCm38/mm10 reference |
| Guinea | Cavia porcellus | February 2008 | Broad/cavPor3 Syntenic net |
| pig | | | |
| Kangaroo | Dipodomys ordii | July 2008 | Broad/dipOrd1 Reciprocal |
| rat | | | best |
| Naked | Heterocephalus | January 2012 | Broad |
| mole-rat | glaber | | HetGla_female_1.0/hetGla2 |
| | | | Syntenic net |
| Pika | Ochotona princeps | July 2008 | Broad/ochPri2 Reciprocal |
| | | | best |
| Rabbit | Oryctolagus cuniculus | April 2009 | Broad/oryCun2 Syntenic net |
| Rat | Rattus norvegicus | March 2012 | RGSC 5.0/rn5 Syntenic net |
| Squirrel | Spermophilus | November 2011 | Broad/speTri2 Syntenic net |
| | tridecemlineatus | | |
| Tree | Tupaia belangeri | December 2006 | Broad/tupBel1 Reciprocal |
| shrew | | | best |
| Marmoset | Callithrix jacchus | March 2009 | WUGSC 3.2/calJac3 |
| | | | Syntenic net |
| Gorilla | Gorilla gorilla | May 2011 | gorGor3 Syntenic net |
| Human | Homo sapiens | February 2009 | GRCh37/hg19 Syntenic net |
| Mouse | Microcebus murinus | June 2003 | Broad/micMur1 Reciprocal |
| lemur | | | best |
| Gibbon | Nomascus leucogenys | June 2011 | GGSC Nleu1.1/nomLeu2 |
| | | | Syntenic net |
| Bushbaby | Otolemur garnettii | March 2011 | Broad/otoGar3 Syntenic net |
| Chimp | Pan troglodytes | February 2011 | Pan_troglodytes- |
| | | | 2.1.4/panTro4 Syntenic net |
| Baboon | Papio hamadryas | November 2008 | Baylor 1.0/papHam1 |
| | | | Reciprocal best |
| Orangutan | Pongo pygmaeus abelii | July 2007 | WUGSC 2.0.2/ponAbe2 |
| | | | Syntenic net |
| Chinese | Macaca mulatta | October 2010 | BGI CR_1.0/rheMac3 |
| rhesus | | | Syntenic net |
| Squirrel | Saimiri boliviensis | October 2011 | saiBol1 Syntenic net |
| monkey | | | |
| Tarsier | Tarsius syrichta | August 2008 | Broad/tarSyr1 Reciprocal |
| | | | best |
| Panda | Ailuropoda melanoleuca | December 2009 | BGI-Shenzhen 1.0/ailMel1 |
| | | | Syntenic Net |
| Cow | Bos taurus | October 2011 | Baylor Btau_4.6.1/bosTau7 |
| | | | Syntenic Net |
| Dog | Canis lupus familiaris | September 2011 | Broad/canFam3 Syntenic net |
| Sloth | Choloepus hoffmanni | July 2008 | Broad//choHof1 Reciprocal |
| | | | best |
| Armadillo | Dasypus novemcinctus | December 2011 | Armadillo/dasNov3 |
| | | | Reciprocal best |
| Tenrec | Echinops telfairi | July 2005 | Broad/echTel1 Reciprocal |
| | | | best |
| Horse | Equus caballus | September 2007 | Broad/equCab2 Syntenic net |
| Hedgehog | Erinaceus europaeus | June 2006 | Broad/eriEur1 Reciprocal |
| | | | best |
| Cat | Felis catus | September 2011 | ISGSC Felis_catus |
| | | | 6.2/felCat5 Reciprocal best |
| Elephant | Loxodonta africana | July 2009 | Broad/loxAfr3 Syntenic net |
| Microbat | Myotis lucifugus | July 2010 | Broad/myoLuc2 Reciprocal |
| | | | best |
| Sheep | Ovis aries | February 2010 | ISGC/oviAri1 Reciprocal |
| | | | best |
| Rock | Procavia capensis | July 2008 | Broad/proCap1 Reciprocal |
| hyrax | | | best |
| Megabat | Pteropus vampyrus | July 2008 | Broad/pteVam1 Reciprocal |
| | | | best |
| Shrew | Sorex araneus | June 2006 | Broad/sorAra1 Reciprocal |
| | | | best |
| Pig | Sus scrofa | August 2011 | SGSC Sscrofa10.2/susScr3 |
| | | | Syntenic net |
| Manatee | Trichechus manatus | October 2011 | Broad v1.0/triMan1 Syntenic |
| | latirostris | | net |
| Dolphin | Tursiops truncatus | October 2011 | Baylor Ttru_1.4/turTru2 |
| | | | Reciprocal best |
| Alpaca | Vicugna pacos | July 2008 | Broad/vicPac1 Reciprocal |
| | | | best |

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

Abramoff, M. D. et al. (2004). J. Biophotonics Int. 11, 36-41. doi:10.1117/1.3589100.
Akimoto, M. et al. (1999). Investig. Ophthalmol. Vis. Sci. 40, 273-279.
Akiyama, H. et al. (2006). J. Cell. Physiol. 207, 407-412. doi:10.1002/jcp.20583.
Akopian, A. et al. (2016). J. Comp. Neurol. doi:10.1002/cne.24074.
Aliç, I. et al. (2016). Neurosci. Lett. 634, 32-41. doi:10.1016/j.neulet.2016.10.001.
Amrite, A. C. et al. (2006). Investig. Ophthalmol. Vis. Sci. 47, 1149-1160. doi:10.1167/iovs.05-0531.
Anderson, C. L. et al. (2006). Mol. Ther. 14, 724-734. doi:10.1016/j.ymthe.2006.04.013.
Antoniou, M. et al. (2003). Genomics 82, 269-279. doi:10.1016/S0888-7543(03)00107-1.
Aoun, P. et al. (2003). Investig. Ophthalmol. Vis. Sci. 44, 2999-3004. doi:10.1167/iovs.02-1060.
Auricchio, A. et al. (2001). Mol. Ther. 4, 372-374. doi:10.1006/mthe.2001.0462.
Baden, T. et al. (2016). Nature 529, 345-350. doi:10.1038/nature16468.
Bainbridge, J. W. B. et al. (2015). N. Engl. J. Med. 372, 1887-97. doi:10.1056/NEJMoa1414221.
Bainbridge, J. W. B. et al. (2008). N. Engl. J. Med. 358, 2231-39. doi:10.1056/NEJMoa0802268.
Balaggan, K. S. et al. (2006). J. Gene Med. 8, 275-285. doi:10.1002/jgm.845.
Barres, B. A. et al. (1988). Neuron 1, 791-803. doi:10.1016/0896-6273(88)90127-4.
Barrett, T. G. et al. (1997). Eye 11, 882-888. doi:10.1038/eye.1997.226.
Bearer, E. L. et al. (2007). Neuroimage 37. doi:10.1016/j.neuroimage.2007.04.053.
Bennett, J. et al. (1996). Nat. Med. 2, 649-654.
Bennett, J. et al. (2016). Lancet 388, 661-672. doi:10.1016/S0140-6736(16)30371-3.
Bennett, J. et al. (1998). Gene Ther. 5, 1156-64. doi:10.1038/sj.gt.3300733.
Bessant, D. A. R. et al. (2001). Curr. Opin. Genet. Dev. 11, 307-316. doi:10.1016/S0959-437X(00)00195-7.
Bikbova, G. et al. (2013). Brain Res. 1534, 33-45. doi:10.1016/j.brainres.2013.08.027.
Bombelli, F. et al. (2014). JAMA Neurol. 71, 1036-42. doi:10.1001/jamaneurol.2014.629.
Bonneau, D. et al. (2014). Brain 137. doi:10.1093/brain/awu184.
Boye, S. E. et al. (2016). Hum. Gene Ther. 27, 580-597. doi:10.1089/hum.2016.085.
Boye, S. E. et al. (2010). PLOS One 5. doi:10.1371/journal.pone.0011306.
Brown, J. M. et al. (1998). Cancer Res. 58, 1408-1416. doi:9537241.
Buch, P. K. et al. (2006). Mol. Ther. 14, 700-709. doi:10.1016/j.ymthe.2006.05.019.
Budanov, A. V et al. (2004). Science 304, 596-600. doi:10.1126/science.1095569.
Busskamp, V. et al. (2010). Science (80-.). 329, 413-417. doi:10.1126/science.1190897.
Callaway, E. M. (2005). J. Physiol. 566, 13-9. doi:10.1113/jphysiol.2005.088047.
Caplen, N. et al. (1995). Gene Ther. 29, 603-13.
Carmignoto, G. et al. (1989). J Neurosci 9, 1263-1272.
Cayouette, M. et al. (1999). Neurobiol. Dis. 6, 523-32. doi:10.1006/nbdi.1999.0263.
Cen, L. P. et al. (2017). Neuroscience 343, 472-482. doi:10.1016/j.neuroscience.2016.12.027.
Chadderton, N. et al. (2009). Mol. Ther. 17, 593-599. doi:10.1038/mt.2008.301.
Chadderton, N. et al. (2012). Eur. J. Hum. Genet., 62-68. doi:10.1038/ejhg.2012.112.
Chalberg, T. W. et al. (2005). Investig. Ophthalmol. Vis. Sci. 46, 2140-2146. doi:10.1167/iovs.04-1252.
Chan, J. W. (2002). Ocul. Immunol. Inflamm. 10, 161-186. doi:10.1076/ocii.10.3.161.15603.
Chavala, S. H. et al. (2005). Eur. J. Intern. Med. 16, 447-448. doi:10.1016/j.ejim.2005.01.021.
Chen, H. et al. (2014). Surv. Ophthalmol. 59, 64-76. doi:10.1016/j.survophthal.2013.02.005.
Chintalapudi, S. R. et al. (2016). Front. Aging Neurosci. 8. doi:10.3389/fnagi.2016.00093.
Chng, K. et al. (2007). J. Gene Med. 9, 22-32. doi:10.1002/jgm.990.
Choi, V. W. et al. (2015). Mol. Ther. Methods Clin. Dev. 2, 15022. doi:10.1038/mtm.2015.22.
Choudhury, S. et al. (2016). Front. Neurosci. 10, 551. doi:10.3389/fnins.2016.00551.
Cronin, T. et al. (2014). EMBO Mol. Med. 6, 1-16. doi:10.15252/emmm.201404077.
Dalkara, D. et al. (2013). Sci. Transl. Med. 5, 189ra76. doi:10.1126/scitranslmed.3005708.
Daly, C. et al. (2016). Histone deacetylase: Therapeutic targets in retinal degeneration. doi:10.1007/978-3-319-17121-0_61.
Dejneka, N. S. et al. (2004). Mol. Ther. 9, 182-188. doi:10.1016/j.ymthe.2003.11.013.
Di Polo, A. et al. (1998). Proc. Natl. Acad. Sci. U.S.A. 95, 3978-83. doi:10.1073/pnas.95.7.3978.
DiCiommo, D. P. et al. (2004). Investig. Ophthalmol. Vis. Sci. 45, 3320-3329. doi:10.1167/iovs.04-0140.
Diester, I. et al. (2011). Nat. Neurosci. 14, 387-397. doi:10.1038/nn.2749.
Donello, J. E. et al. (1998). J. Virol. 72, 5085-92.
Dong, J. Y. et al. (1996). Hum. Gene Ther. 7, 2101-2112. doi:10.1089/hum. 1996.7.17-2101.
Doonan, F. et al. (2011). J. Neurochem. 118, 915-927. doi:10.1111/j.1471-4159.2011.07354.x.
Doonan, F. et al. (2009). J. Neurochem. 109, 631-643. doi:10.1111/j. 1471-4159.2009.05995.x.
Eguchi, T. et al. (2007). Biochimie 89, 278-288. doi:10.1016/j.biochi.2006.12.006.
Faktorovich, E. G. et al. (1990). Nature 347, 83-6. doi:10.1038/347083a0.
Fan, Y. et al. (2008). Arterioscler. Thromb. Vasc. Biol. 28, 315-21. doi:10.1161/ATVBAHA.107.149815.
Farjo, R. et al. (2006). PLOS One 1. doi:10.1371/journal.pone.0000038.
Farrar, G. J. et al. (2013). Trends Genet. 29, 488-497. doi:10.1016/j.tig.2013.05.005.
Farrar, G. J. et al. (2014). Vis. Neurosci. 31, 289-307. doi:10.1017/S0952523814000133.
Featherstone, C. (1993). Am Biotechnol Lab 11.
Feng, L. et al. (2017). eNeuro 4. doi:10.1523/ENEURO.0331-16.2016.
Feuer, W. J. et al. (2015). Ophthalmology, 1-13. doi:10.1016/j.ophtha.2015.10.025.
Fischer, M. D. et al. (2017). Mol. Ther. doi:10.1016/j.ymthe.2017.05.005.
Flannery, J. G. et al. (1997). Proc. Natl. Acad. Sci. U.S.A. 94, 6916-6921. doi:10.1073/pnas.94.13.6916.
Fortuna, F. et al. (2009). Brain 132, 116-123. doi:10.1093/brain/awn269.
Frasson, M. et al. (1999). Investig. Ophthalmol. Vis. Sci. 40, 2724-2734.
Frezza, D. et al. (2007). Ann. . . . 66, 1210-5. doi:10.1136/ard.2006.066597.
Gao, Q. et al. (2007). Brain Res. 1130, 1-16. doi:10.1016/j.brainres.2006.10.018.
Garcia-Frigola, C. et al. (2007). BMC Dev. Biol. 7, 103. doi:10.1186/1471-213X-7-103.
Gaub, B. M. et al. (2014). Proc. Natl. Acad. Sci. U.S.A. 111, E5574-83. doi:10.1073/pnas. 1414162111.
Genzer, M. A. et al. (2007). Nucleic Acids Res. 35, 1178-1186. doi:10.1093/nar/gkm014.
Ghazi, N. G. et al. (2016). Hum. Genet. 135, 327-343. doi:10.1007/s00439-016-1637-y.
Gregory-Evans, K. et al. (2009). Mol. Vis. 15, 962-973.
Grieger, J. C. et al. (2005). J. Virol. 79, 9933-44. doi:10.1128/JVI.79.15.9933-9944.2005.
Griffiths, L. et al. (2000). Gene Ther. 7, 255-62. doi:10.1038/sj.gt.3301058.
Grzybowski, A. et al. (2015). Acta Ophthalmol. 93, 402-410. doi:10.1111/aos.12515.
Hall, K. M. et al. (2006). Exp. Hematol. 34, 433-442. doi:10.1016/j.exphem.2005.12.014.
Hangai, M. et al. (1998). Arch. Ophthalmol. 116, 342-8.
Hattori, Y. et al. (2005). Curr. Drug Deliv. 2, 243-252. doi:10.2174/1567201054368002.
Hauswirth, W. W. et al. (2008). Hum. Gene Ther. 19, 979-90. doi:10.1089/hum.2008.107.
Hayashi, N. et al. (2000). Ophthalmology 107, 1397-1402. doi:10.1016/S0161-6420 (00) 00110-X.
Hoekel, J. et al. (2014). J. AAPOS 18, 461-465.e1. doi:10.1016/j.jaapos.2014.07.162.
Igarashi, T. et al. (2016). Mol. Vis. 22, 816-26.
Ikeda, Y. et al. (2002). Exp. Eye Res. 75, 39-48. doi:10.1006/exer.2002.1177.
Ivanova, E. et al. (2010). Investig. Ophthalmol. Vis. Sci. 51, 5288-5296. doi:10.1167/iovs.10-5389.
Jeon, C. J. et al. (1998). J Neurosci 18, 8936-8946.
Jin, H. et al. (1996). Nat Genet. 14, 353-6. doi:10.1038/ng0496-417.
Joly, S. et al. (2008). J. Neurosci. 28, 13765-13774. doi:10.1523/JNEUROSCI.5114-08.2008.
Ju, J. et al. (2015). Int. J. Mol. Sci. 16, 5666-5681. doi:10.3390/ijms16035666.
Karmali, P. P. et al. (2007). Med. Res. Rev. 27, 696-722. doi:10.1002/med.20090.
Kass, I. et al. (1957). JAMA, 1740-1743.
Katz, B. J. et al. (2006). Am. J. Med. Genet. Part A 140, 2207-2211. doi:10.1002/ajmg.a.31455.
Kay, C. N. et al. (2013). PLOS One 8. doi:10.1371/journal.pone.0062097.
Kent, W. J. et al. (2002). Genome Res. 12, 996-1006. doi:10.1101/gr.229102.
Kerrison, J. B. et al. (1999). Arch Ophthalmol 117, 805-810.
Khalilpour, S. et al. (2017). J. Neurol. Sci. 375, 430-441. doi:10.1016/j.jns.2016.12.044.
Khandekar, M. et al. (2007). Development 134, 1703-1712. doi:10.1242/dev.001297.
Khani, S. C. et al. (2007). Investig. Ophthalmol. Vis. Sci. 48, 3954-3961. doi:10.1167/iovs.07-0257.
Kiang, A. S. et al. (2005). Mol. Ther. 12, 555-561. doi:10.1016/j.ymthe.2005.03.028.
Kicic, A., et al. (2003). J. Neurosci. 23, 7742-7749. doi:23/21/7742 [pii].
Kidd, D. P. (2016). Neurol. Neuroimmunol. neuroinflammation 3, e270. doi:10.1212/NXI.0000000000000270.
Kim, C. Y. (2006). Mol. Vis. 12, 1640-8.
Kimchi-Sarfaty, C. et al. (2002). Hum. Gene Ther. 13, 299-310. doi:10.1089/10430340252769815.
Kimura, A. et al. (2016). Int. J. Mol. Sci. 17. doi:10.3390/ijms17091584.
Koeberle, P. D. et al. (2002). Neuroscience 110, 555-567. doi:S0306452201005577 [pii].
Koilkonda, R. D. et al. (2010). Arch. Ophthalmol. (Chicago, Ill. 1960) 128, 876-83. doi:10.1001/archophthalmol.2010.135.
Koilkonda, R. D. et al. (2014). JAMA Ophthalmol. 33136, 409-420. doi:10.1001/jamaophthalmol.2013.7630.
Kornguth, S. et al. (1981). Neurosci. Lett. 27, 151-157. doi:10.1016/0304-3940 (81) 90260-3.
Kügler, S. et al. (2003a). Gene Ther. 10, 337-347. doi:10.1038/sj.gt.3301905.
Kügler, S. et al. (2003b). Virology 311, 89-95. doi:10.1016/S0042-6822(03)00162-4.
Kwaks, T. H. J. et al. (2006). Trends Biotechnol. 24, 137-142. doi:10.1016/j.tibtech.2006.01.007.
Kyrchanova, O. et al. (2007). Mol. Cell. Biol. 27, 3035-43. doi:10.1128/MCB.02203-06.
Lau, D. et al. (2000). Investig. Ophthalmol. Vis. Sci. 41, 3622-3633.
Lebherz, C. et al. (2008). J. Gene Med. 10, 375-382. doi:10.1002/jgm.1126.
Lebkowski, J. S. et al. (1988). Mol. Cell. Biol. 8, 3988-3996. doi:10.1128/MCB.8.10.3988.
Lee, J. et al. (2016). Theranostics 6, 192-203. doi:10.7150/thno.13657.
Lenaers, G. et al. (2012). Orphanet J. Rare Dis. 7, 46. doi:10.1186/1750-1172-7-46.
Léveillard, T. et al. (2004). Nat. Genet. 36, 755-9. doi:10.1038/ng1386.
Li, Q., Miller et al. (2008). Mol. Vis. 14, 1760-9.
Li, R. et al. (2011). PLOS One 6. doi:10.1371/journal.pone.0023148.
Li, S. Y. et al. (2010). Int. J. Mol. Sci. 11, 2109-2117. doi:10.3390/ijms11052109.
Liao, H. W. et al. (2007). Biotechniques 76, 211-220. doi:10.1007/s11103-011-9767-z. Plastid.
Lin, Y.-J. et al. (1997). Magn. Reson. Med. 38, 378-388. doi:10.1002/mrm. 1910380305.
Lindsey, J. D. et al. (2007). Neuroimage 34, 1619-1626. doi:10.1016/j.neuroimage.2006.07.048.
Lipps, B. V (2002). J. Nat. Toxins 11, 57-62.
Liu, T., et al. (2006). Cancer Treat Rev 32, 157-165. doi:10.1016/j.ctrv.2005.12.006.
Liu, T. et al. (2012). J. Biomed. Mater. Res.-Part A 100 A, 236-242. doi:10.1002/jbm.a.33271.
Lopez, A. J. et al. (2016). J. Neurosci. 36, 3588-3599. doi:10.1523/JNEUROSCI.3682-15.2016.
Lu, H.-X. et al. (2011). Med. Sci. Monit. 17, BR305-311.
Lu, Y. et al. (2012). Adv. Drug Deliv. Rev. 64, 342-352. doi:10.1016/j.addr.2012.09.020.
MacLaren, R. E. et al. (2014). Lancet 383, 1129-1137. doi:10.1016/S0140-6736(13)62117-0.
Mao, Y. et al. (2016). BMC Biotechnol. 16, 1. doi:10.1186/s12896-015-0230-0.
Marano, R. J. et al. (2005). Gene Ther. 12, 1544-50. doi:10.1038/sj.gt.3302579.
Masland, R. H. (2012). Neuron 76, 266-280. doi:10.1016/j.neuron.2012.10.002.
Matsuda, T., et al. (2004). Proc. Natl. Acad. Sci. U.S.A. 101, 16-22. doi:10.1073/pnas.2235688100.
McKernan, D. P. et al. (2007). Invest. Ophthalmol. Vis. Sci. 48, 5420-5430. doi:10.1167/iovs.07-0287.
McLaren, M. J. et al. (1997). FEBS Lett. 412, 21-29. doi:10.1016/S0014-5793(97)00566-8.
McNally, N. et al. (2002). Hum. Mol. Genet. 11, 1005-16. doi:10.1093/hmg/11.9.1005.
Miller, N. R. (2004). Eye (Lond). 18, 1026-37. doi:10.1038/sj.eye.6701592.
Millington-Ward, S. et al. (2011). Mol. Ther. 19, 642-649. doi:10.1038/mt.2010.293.
Molday, L. L. et al. (2013). Hum. Mol. Genet. 22, 3894-3905. doi:10.1093/hmg/ddt244.
Montezuma, S. R. et al. (2006). Investig. Ophthalmol. Vis. Sci. 47, 3514-3522. doi:10.1167/iovs.06-0106.
Mowat, F. M. et al. (2014). Gene Ther. 21, 96-105. doi:10.1038/gt.2013.64.
Mueller, C. et al. (2008). Gene Ther. 15, 858-63. doi:10.1038/gt.2008.68.
Nadal-Nicolás, F. M. et al. (2009). Investig. Ophthalmol. Vis. Sci. 50, 3860-3868. doi:10.1167/iovs.08-3267.
Nadal-Nicolás, F. M. et al. (2014). Front. Neuroanat. 8, 99. doi:10.3389/fnana.2014.00099.
Ng, T. F. et al. (2007). Chem. Immunol. Allergy 92, 300-316. doi:10.1159/000099280.
Noval, S. et al. (2012). Eye 26, 315-320. doi:10.1038/eye.2011.291.
O'Reilly, M. et al. (2007). Am. J. Hum. Genet. 81, 127-135. doi:10.1086/519025.
Ortín-Martínez, A. et al. (2014). PLOS One 9. doi:10.1371/journal.pone.0113798.
Palfi, A. et al. (2006). Hum. Mutat. 27, 260-268. doi:10.1002/humu.20287.
Palfi, A. et al. (2012). Hum. Gene Ther. 23, 847-858. doi:10.1089/hum.2011.142.
Palfi, A. et al. (2015). Mol. Ther. Methods Clin. Dev. 2, 15016. doi:10.1038/mtm.2015.16.
Palfi, A. et al. (2010). Hum. Gene Ther. 21, 311-323. doi:10.1089/hum.2009.119.
Pang, C. P. et al. (2006). Mol. Vis. 12, 85-92. doi:v12/a9 [pii].
Passman, R. S. et al. (2012). Am. J. Med. 125, 447-453. doi:10.1016/j.amjmed.2011.09.020.
Patricio, M. I. et al. (2017).Mol. Ther.-Nucleic Acids 6, 198-208. doi:10.1016/j.omtn.2016.12.006.
Petrs-Silva, H. et al. (2009). Mol. Ther. 17, 463-471. doi:10.1038/mt.2008.269.
Prusky, G. T. et al. (2004). Investig. Ophthalmol. Vis. Sci. 45, 4611-4616. doi:10.1167/iovs.04-0541.
Purvin, V., et al. (2009). Clin. Exp. Ophthalmol. 37, 712-717. doi:10.1111/j.1442-9071.2009.02122.x.
Purvin, V. et al. (2011). J. Neuro-Ophthalmology 31, 58-68. doi:10.1097/WNO.0b013e31820cf78a.
Ramachandran, P. et al. (2016). Hum. Gene Ther., hum.2016.111. doi:10.1089/hum.2016.111.
Rathnasamy, G. et al. (2017). Mol. Neurobiol. 54, 3453-3464. doi:10.1007/s12035-016-9905-3.
Reed, S. E. et al. (2006). J. Virol. Methods 138, 85-98. doi:10.1016/j.jviromet.2006.07.024.
Rex, T. S. et al. (2009). Exp. Eye Res. 89, 735-740. doi:10.1016/j.exer.2009.06.017.
Reynier, P. et al. (2004). J. Med. Genet. 41, e110. doi:10.1136/jmg.2003.016576.
Rhee, K. Do et al. (2010). Advances in Experimental Medicine and Biology, 647-654. doi:10.1007/978-1-4419-1399-9_74.
Rohr, U.-P. et al. (2002). J. Virol. Methods 106, 81-8. doi:10.1016/S0166-0934(02)00138-6.
Rong, X. et al. (2011). Eur. J. Pharm. Sci. 43, 334-42. doi:10.1016/j.ejps.2011.05.011.
Russell, S. et al. (2017). Lancet 6736, 1-12. doi:10.1016/S0140-6736(17)31868-8.
Sakurai, F. et al. (2001). Gene Ther. 8, 677-86. doi:10.1038/sj.gt.3301460.
Schambach, A. et al. (2006). Gene Ther. 13, 641-645. doi:10.1038/sj.gt.3302698.
Schlamp, C. L. et al. (2013). Mol. Vis. 19, 1387-96.
Sengupta, A. et al. (2016). EMBO Mol. Med. 8, 1248-1264. doi:10.15252/emmm.201505699.
Shoge, K. et al. (1999). Neurosci. Lett. 259, 111-114.
Sieving, P. A. et al. (2006). Proc. Natl. Acad. Sci. U.S.A. 103, 3896-901. doi:10.1073/pnas.0600236103.
So, N. M. et al. (2000). Acta Radiol. 41, 559-61.
Spanopoulou, E. et al. (1991). Mol. Cell. Biol. 11, 2216-28. doi:10.1128/MCB.11.4.2216.Updated.
Struebing, F. L. et al. (2016). Front. Genet. 7, 1-14. doi:10.3389/fgene.2016.00169.
Sullivan, T. A. et al. (2011). Hum. Gene Ther. 22, 1191-1200. doi:10.1089/hum.2011.052.
Sun, X., et al. (2010). Gene Ther. 17, 117-31. doi:10.1038/gt.2009.104.
Suri, D. et al. (2016). Lupus 25, 93-96. doi:10.1177/0961203315603142.
Takahashi, M. (2004). Methods Mol Biol 246, 439-449.
Takazawa, T. et al. (2014). Orbit 33, 13-6. doi:10.3109/01676830.2013.841716.
Tian, L. et al. (2005). J. Plant Physiol. 162, 1355-1366. doi:10.1016/j.jplph.2005.03.011.
Tomita, H. et al. (2005). Cardiovasc. Res. 67, 134-41. doi:10.1016/j.cardiores.2005.02.022.
Tönges, L. et al. (2011). J. Neurochem. 117, 892-903. doi:10.1111/j.1471-4159.2011.07257.x.
Trompeter, H.-I. I. et al. (2003). J. Immunol. Methods 274, 245-256. doi:10.1016/S0022-1759(02)00431-3.
Trost, A. et al. (2015). Exp. Eye Res. 136, 59-71. doi:10.1016/j.exer.2015.05.010.
Trujillo, C. A. et al. (2007). Clin. Ophthalmol. 1, 393-402.
Trujillo, M. A. et al. (2006). Mol. Endocrinol. 20, 2559-75. doi:10.1210/me.2006-0147.
TSAI, R. et al. (1997). J. Ocul. Pharmacol. Ther. 13, 473-477.
Tshilenge, K.-T. et al. (2016). Hum. Gene Ther. Methods 27, 122-134. doi:10.1089/hgtb.2016.034.
Usui, S. et al. (2009). Mol. Ther. 17, 778-86. doi:10.1038/mt.2009.47.
Uteza, Y. et al. (1999). Proc. Natl. Acad. Sci. U.S.A. 96, 3126-3131. doi:10.1073/pnas.96.6.3126.
Ververis, K., et al. (2013). Biol. Targets Ther. 7, 47-60. doi:10.2147/BTT.S29965.
Wang, Q. et al. (2011). Cell. Physiol. Biochem. 27, 769-782. doi:10.1159/000330085.
Wassle, H. J. Comp. Neurol. 265, 391-408. doi:10.1002/cne.902650308.
Wassmer, S. J. et al. (2017). Sci. Rep. 7, 45329. doi:10.1038/srep45329.
Watanabe, T. et al. (2001). Magn. Reson. Med. 46, 424-429. doi:10.1002/mrm.1209.
Weinreb, R. N. et al. (2014). JAMA 311, 1901-11. doi:10.1001/jama.2014.3192.
Weinshenker, B. G. et al. (2017). Mayo Clin. Proc. 92, 663-679. doi:10.1016/j.mayocp.2016.12.014.
Welsbie, D. S. et al. (2017). Neuron 94, 1142-1154.e6. doi:10.1016/j.neuron.2017.06.008.
Wert, K. J. et al. (2013). Hum. Mol. Genet. 22, 558-567. doi:10.1093/hmg/dds466.
Williams, S. et al. (2005). BMC Biotechnol. 5, 17. doi:10.1186/1472-6750-5-17.
Wu, W.-C. et al. (2004). Mol. Vis. 10, 93-102. doi:v10/a13 [pii].
Xiang, M. et al. (1996). Proc Natl Acad Sci USA 93, 596-601.
Xiao, X. et al. (1998). J. Virol. 72, 2224-32. doi:10.1073/pnas.1201800109.
Yang, S. et al. (2016). EBioMedicine 10, 258-268. doi:10.1016/j.ebiom.2016.07.002.
Yin, L. et al. (2011). Investig. Ophthalmol. Vis. Sci. 52, 2775-2783. doi:10.1167/iovs.10-6250.
Ying, S. et al. (1998). Curr. Eye Res., 777-782. doi:10.1076/ceyr.17.8.777.5158.
Yu-Wai-Man, P. et al. (2016). Acta Neuropathol. 132, 789-806. doi:10.1007/s00401-016-1625-2.
Zeng, Y. et al. (2007). J. Virol. 81, 2401-2417. doi:10.1128/JVI.02024-06.
Zhang, Y. et al. (2009). J. Neurosci. 29, 9186-9196. doi:10.1523/JNEUROSCI.0184-09.2009.
Zhao, X. et al. (2006). Brain Res. 1073-1074, 460-469. doi:10.1016/j.brainres.2005.12.061.
Zhu, Y. et al (2014). J. Neurosci. 34, 7845-61. doi:10.1523/JNEUROSCI.2960-13.2014.
Zolotukhin, S. et al. (1999). Gene Ther. 6, 973-985. doi:10.1038/sj.gt.3300938.

We claim:

1. A method of treatment of ocular disease, wherein said method comprises administering to an eye an isolated nucleic acid molecule having promoter activity, wherein said nucleic acid molecule comprises Neurofilament heavy gene promoter conserved region A, but does not comprise any of Neurofilament heavy gene promoter conserved regions D, D1, K, B, C and E;
wherein Neurofilament heavy gene promoter conserved region A is a nucleotide sequence having the nucleotide sequence shown as SEQ ID NO: 1, or a functional variant thereof; Neurofilament heavy gene promoter conserved region D is a nucleotide sequence having the nucleotide sequence shown as SEQ ID NO: 2, or a functional variant thereof; Neurofilament heavy gene promoter conserved region D1 is a nucleotide sequence having the nucleotide sequence shown as SEQ ID NO: 4, or a functional variant thereof; Neurofilament heavy gene promoter conserved region K is a nucleotide sequence having the nucleotide sequence shown as SEQ ID NO: 5, or a functional variant thereof; Neurofilament heavy gene promoter conserved region B is a nucleotide sequence having the nucleotide sequence shown as SEQ ID NO: 6, or a functional variant thereof; Neurofilament heavy gene promoter conserved region C is a nucleotide sequence having the nucleotide sequence shown as SEQ ID NO: 7, or a functional variant thereof; and Neurofilament heavy gene promoter conserved region E is a nucleotide sequence having the nucleotide sequence shown as SEQ ID NO: 8, or a functional variant thereof.

2. The method according to claim 1, wherein said nucleic acid molecule further comprises Neurofilament heavy gene promoter conserved region F, wherein said Neurofilament heavy gene promoter conserved region F is a nucleotide sequence having the nucleotide sequence shown as SEQ ID NO: 3 or a functional variant thereof.

3. The method according to claim 2, wherein the isolated nucleic acid molecule does not comprise a spacer sequence between conserved regions A and F.

4. The method according to claim 1, wherein a functional variant of a recited nucleotide sequence is a variant that can hybridize under stringent hybridization conditions (0.2×SSC, 0.1% SDS) to, or that shares at least 70% identity with, the recited nucleotide sequence.

5. The method according to claim 1, wherein the full length NEFH promoter sequence consists of the nucleic acid sequence shown as SEQ ID NO: 21, SEQ ID No: 22 or SEQ ID NO:23.

6. The method according to claim 5, wherein the full length NEFH promoter sequence consists of the nucleic acid sequence shown as SEQ ID NO: 21.

7. The method according to claim 5, wherein the full length NEFH promoter sequence consists of the nucleic acid sequence shown as SEQ ID NO: 22.

8. The method according to claim 1, wherein the isolated nucleic acid molecule comprises Neurofilament heavy gene promoter conserved regions A and F having the nucleic acid sequences shown as SEQ ID NOS: 10 and 16 respectively.

9. The method according to claim 1, wherein said nucleic acid molecule having promoter activity provides preferential expression of one or more heterologous polynucleotide sequences to which it is operably linked in the ganglion cell layer of the eye.

10. The method according to claim 1, wherein the ocular disease is Leber Hereditary Optic Neuropathy (LHON), dominant optic atrophy (DOA), or glaucoma.

11. An isolated nucleic acid molecule having promoter activity, wherein said nucleic acid molecule comprises Neurofilament heavy gene promoter conserved region A and Neurofilament heavy gene promoter conserved region F, but does not comprise any of Neurofilament heavy gene promoter conserved regions D, D1, K, B, C and E;
wherein Neurofilament heavy gene promoter conserved region A is a nucleotide sequence having the nucleotide sequence shown as SEQ ID NO: 1, or a functional variant thereof; Neurofilament heavy gene promoter conserved region D is a nucleotide sequence having the nucleotide sequence shown as SEQ ID NO: 2, or a functional variant thereof; Neurofilament heavy gene promoter conserved region F is a nucleotide sequence having the nucleotide sequence shown as SEQ ID NO: 3, or a functional variant thereof; Neurofilament heavy gene promoter conserved region D1 is a nucleotide sequence having the nucleotide sequence shown as SEQ ID NO: 4, or a functional variant thereof; Neurofilament heavy gene promoter conserved region K is a nucleotide sequence having the nucleotide sequence shown as SEQ ID NO: 5, or a functional variant thereof; Neurofilament heavy gene promoter conserved region B is a nucleotide sequence having the nucleotide sequence shown as SEQ ID NO: 6, or a functional variant thereof; Neurofilament heavy gene promoter conserved region C is a nucleotide sequence having the nucleotide sequence shown as SEQ ID NO: 7, or a functional variant thereof; and Neurofilament heavy gene promoter conserved region E is a nucleotide sequence having the nucleotide sequence shown as SEQ ID NO: 8, or a functional variant thereof.

12. The isolated nucleic acid molecule according to claim 11, wherein the isolated nucleic acid molecule does not comprise a spacer sequence between conserved regions A and F.

13. The isolated nucleic acid molecule according to claim 11, wherein a functional variant of a recited nucleotide sequence is a variant that can hybridize under stringent hybridization conditions (0.2×SSC, 0.1% SDS) to, or that shares at least 70% identity with, the recited nucleotide sequence.

14. The isolated nucleic acid molecule according to claim 11, wherein the full length NEFH promoter sequence consists of the nucleic acid sequence shown as SEQ ID NO: 21, SEQ ID No:22 or SEQ ID NO:23.

15. The isolated nucleic acid molecule according to claim 14, wherein the full length NEFH promoter sequence consists of the nucleic acid sequence shown as SEQ ID NO: 21.

16. The isolated nucleic acid molecule according to claim 14, wherein the full length NEFH promoter sequence consists of the nucleic acid sequence shown as SEQ ID NO: 22.

17. The isolated nucleic acid molecule according to claim 11, wherein the isolated nucleic acid molecule comprises Neurofilament heavy gene promoter conserved regions A and F having the nucleic acid sequences shown as SEQ ID NOS: 10 and 16 respectively.

18. The isolated nucleic acid molecule according to claim 11, wherein said nucleic acid molecule having promoter activity provides preferential expression of one or more heterologous polynucleotide sequences to which it is operably linked in the ganglion cell layer of the eye.

19. A vector comprising the isolated nucleic acid according to claim 11.

20. A therapeutic composition comprising the isolated nucleic acid molecule according to claim 11.
